(19) United States
(12) Patent Application Publication
(10) Pub. No.: US 2023/0057355 A1
(43) Pub. Date: Feb. 23, 2023

(54) GENE NETWORKS THAT MEDIATE REMYELINATION OF THE HUMAN BRAIN

(71) Applicant: UNIVERSITY OF ROCHESTER, Rochester, NY (US)

(72) Inventors: Steven A. GOLDMAN, Webster, NY (US); John MARIANI, Rochester, NY (US)

(21) Appl. No.: 17/430,768

(22) PCT Filed: Feb. 11, 2020
(86) PCT No.: PCT/US2020/017737

§ 371 Date: Aug. 13, 2021

Related U.S. Application Data
(60) Provisional application No. 62/805,202, filed on Feb. 13, 2019.

(51) Int. Cl. A61K 35/30; A61K 45/06; C12N 5/079; A61P 25/00

(52) U.S. Cl. CPC A61K 35/30; A61P 25/00; C12N 5/0622; A61K 45/06

(57) ABSTRACT

The present disclosure relates to methods of treating a human subject having a condition mediated by a deficiency in myelin and methods of increasing oligodendrocyte production from human glial progenitor cells. These methods involve selecting a human subject having a condition mediated by a deficiency in myelin or providing a population of human glial progenitor cells and administering to the subject or the population of human glial progenitor cells one or modulators of a cell signaling pathway selected from the group consisting of Notch signaling, cAMP signaling, CIP2A signaling, RXRA signaling, TCF7L2 signaling, and combinations thereof under conditions effective to treat the condition or increase oligodendrocyte production.

This application claims the benefit of U.S. Provisional Patent Application Ser. No. 62/805,202, filed Feb. 13, 2019, which is hereby incorporated by reference in its entirety.

This invention was made with government support under NS075345 awarded by National Institutes of Health. The government has certain rights in the invention.

### FIELD

The present application relates to gene networks that mediate remyelination of the human brain.

### BACKGROUND

Oligodendrocytes are the sole source of myelin in the adult CNS, and their loss or dysfunction is at the heart of a wide variety of diseases of both children and adults. In children, the hereditary leukodystrophies accompany cerebral palsy as major sources of demyelination-associated neurological morbidity. In adults, demyelination contributes not only to diseases as diverse as multiple sclerosis and white matter stroke, but also to a broad variety of neurodegenerative and neuropsychiatric disorders (Lee et al., “Oligodendroglia Metabolically Support Axons and Contribute to Neurodegeneration,” Nature 487:443-448 (2012); Roy et al., “Progenitor Cells of the Adult White Matter,” In Myelin Biology and Disorders, R. Lazzarini, ed. (Amsterdam: Elsevier), pp. 259-287 (2004); Tkachev et al., “Oligodendrocyte Dysfunction in Schizophrenia and Bipolar Disorder,” Lancet 362:798-805 (2003)). As a result, the demyelinating diseases are especially attractive targets for cell-based therapeutic strategies. Prior studies have established the potential of neonatally-transplanted fetal human glial progenitor cells (hGPCs) to myelinate the congenitally-hypomyelinated brain, and to rescue the neurological phenotype of treated animals (Windrem et al., “Fetal and Adult Human Oligodendrocyte Progenitor Cell Isolates Myelinate the Congenitally Dysmyelinated Brain,” Nat. Med. 10:93-97 (2004); Windrem et al., “Neonatal Chimerization with Human Glial Progenitor Cells Can Both Remyelinate and Rescue Otherwise Lethally Hypomyelinated Shiverer Mouse,” Cell Stem Cell 2:553-565 (2008)). However, these enriched preparations of human GPCs—also referred to interchangeably as either oligodendrocyte progenitor cells (OPCs) or NG2 cells (Nishiyama et al., “Polydendrocytes (NG2 Cells): Multifunctional Cells with Lineage Plasticity,” Nat. Rev. Neurosci. 10:9-22 (2009))—have not been intensively assessed in demyelinated adult brain tissue; prior attempts at remyelination in adult-demyelinated hosts using hGPCs had been restricted to adult-derived GPCs (Windrem et al., “Progenitor Cells Derived From the Adult Human Subcortical White Matter Disperse and Differentiate as Oligodendrocytes Within Demyelinated Lesions of the Rat Brain,” J. Neurosci. Res. 69:966-975 (2002)), which manifest little expansion or migration when implanted into adult brain, thus limiting their therapeutic utility. Indeed, no previous study has systematically assessed the ability of fetal human GPCs to either migrate within or remyelinate adult-demyelinated brain tissue.

The present disclosure is directed to overcoming these and other deficiencies in the art.

### SUMMARY

A first aspect of the present application relates to a method of treating a human subject having a condition mediated by a deficiency in myelin. This method involves selecting a human subject having a condition mediated by a deficiency in myelin and administering to the selected subject one or more modulators of a cell signaling pathway selected from the group consisting of Notch signaling, cAMP signaling, CIP2A signaling, RXRA signaling, TCF7L2 signaling, and combinations thereof under conditions effective to treat the condition.

Another aspect of the present application relates to a method of increasing oligodendrocyte production from human glial progenitor cells. This method involves providing a population of human glial progenitor cells and administering in vitro to the provided population of human glial progenitor cells, one or more modulators of a cell signaling pathway selected from the group consisting of Notch signaling, cAMP signaling, CIP2A signaling, RXRA signaling, TCF7L2 signaling, and combinations thereof under conditions effective to increase oligodendrocyte production compared to oligodendrocyte production absent said administering.

Given the marked differences between fetal and adult GPCs in their behavior upon neonatal delivery, in which fetal cells have proven more migratory and proliferative than their adult counterparts, it has been investigated whether fetal human GPCs might exhibit sufficient migration and expansion competence in the adult environment to serve as therapeutic vectors for acquired adult-onset demyelination. In this regard, a number of recent studies have supported the readiness with which axons can remyelinate after either congenital or acquired demyelination, if provided myelinogenic cells (Duncan et al., “Extensive Remyelination of the CNS Leads to Functional Recovery,” Proc Natl Acad Sci USA 106:6832-6836 (2009); Piao et al., “Human Embryonic Stem Cell-Derived Oligodendrocyte Progenitors Remyelinate the Brain and Rescue Behavioral Deficits Following Radiation,” Cell Stem Cell 16:98-210 (2015); Windrem et al., “Neonatal Chimerization with Human Glial Progenitor Cells can both Remyelinate and Rescue the Otherwise Lethally Hypomyelinated Shiverer Mouse,” Cell Stem Cell 2:553-565 (2008), which are hereby incorporated by reference in their entirety). On that basis, it has been investigated if human GPCs could remyelinate following diffuse demyelination, as might be encountered clinically in multiple sclerosis and other causes of multicentric adult demyelination. In particular, it was determined if hGPCs delivered directly into the adult brain, could remyelinate adult axons that were either congenitally hypomyelinated, or which became acutely demyelinated in adulthood.

To that end, three distinct experimental paradigms were used. First, it was determined if hGPCs could effectively disperse within and myelinate the adult shiverer brain. By this means, the ability of hGPCs to restore myelin to the congenitally hypomyelinated adult brain was assessed, as might be encountered in the postnatal treatment of a hypomyelinating leukodystrophy. Second, it was then determined if neonatally-engrafted hGPCs could respond to cuprizone-induced adult demyelination by generating new oligodendrocytes and myelinating demyelinated axons, so as to assess the ability of already-resident hGPCs to remyelinate previously-myelinated axons, as might be demanded after acquired demyelination. Third, it was then asked if hGPCs transplanted into the adult brain, after cuprizone demyelination, could remyelinate denuded axons, as might be anticipated in the cell-based treatment of disorders such as progressive multiple sclerosis.

It was found that in each of these experimental paradigms the hGPCs, whether engrafted neonatally or transplanted into adults, effectively dispersed throughout the forebrains, differentiated as oligodendroglia and myelinated demyelinated axons. These data suggest that transplanted hGPCs are competent to disperse broadly and differentiate as myelinogenic cells in the adult brain, and critically, that they are able to remyelinate previously myelinated axons that have experienced myelin loss. On that basis, it was also asked what the transcriptional concomitants of demyelination-associated mobilization might be in resident hGPCs. To that end, hGPCs were isolated from neonatally-chimerized brains after the cessation of cuprizone demyelination, and RNA-seq analysis was used to define those genes and cognate pathways induced by antecedent cuprizone demyelination. Together, these studies establish an operational rationale for assessing the ability of hGPCs to remyelinate demyelinated lesions of the adult human brain, while providing a promising set of molecular targets for the modulation of this process in human cells.

### BRIEF DESCRIPTION OF THE DRAWINGS

FIGS. 1A-1K show human GPCs mediate robust myelination after transplantation into the adult shiverer brain. hGPCs proved both highly migratory and robustly myelinogenic, after delivery at 4-6 weeks of age to the hypomyelinated adult shiverer brain. FIG. 1A shows that by 19-20 weeks of age—13-15 weeks after transplant—the injected cells had dispersed as broadly as is typically observed in similarly transplanted shiverer neonates (Windrem et al., “Neonatal Chimerization with Human Glial Progenitor Cells can both Remyelinate and Rescue the Otherwise Lethally Hypomyelinated Shiverer Mouse,” Cell Stem Cell 2:553-565 (2008); Windrem et al., “A Competitive Advantage by Neonatally Engrafted Human Glial Progenitors Yields Mice Whose Brains are Chimeric for Human Glia,” The Journal of Neuroscience: The Official Journal of the Society for Neuroscience 34:16153-16161 (2014), which are hereby incorporated by reference in their entirety), with a near-uniform distribution of donor cells noted throughout the forebrain white matter. FIG. 1B shows hGPCs delivered to myelin wild-type rag2^{-/-} mice distributed throughout both gray and white matter, though with less mitotic expansion than that noted in hypomyelinated shiverer recipients. FIG. 1C shows that oligodendrocyte differentiation and myelinogenesis by donor hGPCs was robust, with dense myelination of the corpus callosum and fimbria. FIG. 1D is a higher power image of FIG. 1C and shows the high proportion of donor cells in the now humanized host white matter. FIG. 1E shows that while both shiverer and myelin wild-type recipients exhibited substantial donor hGPC colonization after adult transplantation, the callosal densities of all human cells (FIG. 1E) and PDGFαR-defined hGPCs (FIG. 1F) were significantly higher in shiverer rather than myelin wild-type recipients. In the myelin wild-type recipients, the densities of all donor cells and identified hGPCs (FIGS. 1E-1F) were not significantly different between mice killed at 5 and 12 months of age, suggesting that donor cell expansion in the myelin wild-type brain occurred within the first 3 months after adult transplant, by 5 months of age. FIG. 1G shows the density of transferrin (TF)-defined human oligodendroglia was 5-10-fold higher in adult-transplanted shiverers than in myelin wild-type hosts, when both were assessed 3 months after graft, at 5 months of age. FIG. 1H shows a smaller proportion of the donor cell population matured as GFAP-defined astrocytes; these too proved significantly more abundant in the shiverer than wild-type hosts. FIGS. 1I-1K are representative images of anti-human NG2-defined donor-derived hGPCs (FIG. 1I), anti-human GFAP-defined astrocytes (FIG. 1J), and transferrin/human nuclear antigen co-expressing donor-derived oligodendrocytes (FIG. 1K) in 19-week old shiverer white matter, 13 weeks after transplantation at 6 weeks of age. Scale: FIGS. 1C-1D: 100 μm, FIGS. 1I-1K, 50 μm.

FIGS. 2A-2M show hGPCs differentiate as myelinogenic oligodendroglia in response to cuprizone demyelination. FIG. 2A is a schematic that outlines the experimental design for neonatal engraftment followed by adult demyelination. Mice were transplanted with 2×10⁵ hGPCs perinatally, maintained on a control diet through 17 weeks of age, then placed on either a cuprizone-supplemented or normal diet for 12 weeks, then either sacrificed or returned to standard diet and killed at later time-points. FIGS. 2B-2C are serial coronal sections comparing dot-mapped distributions of human (human nuclear antigen, hN) cells in control (FIG. 2B) and cuprizone-fed (FIG. 2C) mice at 49 weeks of age, following 20 weeks recovery on control diet. FIGS. 2D-2G show the relative positions and abundance of human and mouse transferrin (TF)-defined oligodendrocytes, mapped in 20 μm coronal sections of corpus callosa of mice engrafted with hGPCs neonatally, demyelinated as adults from 17-29 weeks of age, then assessed either: FIG. 2D, at the end of the cuprizone diet; FIG. 2E, 8 weeks after return to control diet; or FIG. 2F, 20 weeks after cuprizone cessation. FIG. 2G shows an untreated control, age-matched to FIG. 2F. FIG. 2H shows that the density of human cells in the corpus callosum increases to a greater degree and more rapidly in cuprizone-demyelinated brains than in untreated controls, including during the 12 week period of cuprizone treatment (indicated in gray). FIG. 2I shows that, by 8 weeks after the termination of cuprizone exposure, the density of human oligodendroglia was >5-fold greater in cuprizone-demyelinated than untreated control brains. FIG. 2J shows that by that 8 week recovery point, over half of all hGPCs engrafted in the corpus callosa of cuprizone-treated mice had differentiated as oligodendrocytes, and accordingly (FIG. 2K), over half of all transferrin-defined callosal oligodendrocytes were human; in contrast, relatively few human oligodendrocytes were noted in untreated chimeric brains. FIG. 2L shows substantial colonization by human glia evident in this remyelinated corpus callosum, after 20 week recovery (human nuclear antigen; myelin basic protein). FIG. 2M shows chimeric white matter populated, after cuprizone demyelination, by human GPC-derived oligodendroglia. Anti-human nuclear antigen (hNA)), transferrin; inset highlights relative abundance of hNA+/transferrin+ human oligodendroglia. Scale: FIG. 2L, 100 μm; FIG. 2M, 50 μm, inset, 25 μm.

FIGS. 3A-3K show hGPCs differentiate and remyelinate axons after transplant into adult-demyelinated brain. FIG. 3A shows that, at 6 weeks of age, experimental mice were put on a diet containing 0.2% cuprizone, while litter-mate controls remain on standard diet. At 10 weeks, 4 weeks into a 20 week cuprizone course, the mice were transplanted with 2×10⁵ hGPCs. Mice were sacrificed for histology either at the end of the cuprizone course (at 26 weeks) or after an additional 20-week recovery period (at 46 weeks). FIGS. 3B-3C are maps that show locations of individual human cells in 20 μm coronal hemi-sections of engrafted brains. FIG. 3B shows transplantation of hGPCs into a normally-myelinated 10-week old mouse yielded widespread engraftment, when mapped 36 weeks later at 46 weeks of age. FIG. 3C shows that, in cuprizone-treated mice, transplanted hGPCs expanded to a significantly greater degree. FIGS. 3D-3H show significantly more hGPCs differentiated as transferrin (TF)-defined oligodendrocytes in the cuprizone-demyelinated brains than in their untreated controls. FIG. 3D shows hGPCs were more likely to differentiate as transferrin-expressing oligodendrocytes when transplanted into a demyelinating environment (left), compared to a control brain (right). FIGS. 3E-3F show the absolute density (FIG. 3E) and relative proportion (FIG. 3F) of human cells that differentiated as transferrin+oligodendrocytes in the corpus callosum were respectively >5- and >10-fold greater in mice on the cuprizone diet than in their untreated controls. FIG. 3G shows that, by 36 weeks posttransplant, over a quarter of all oligodendrocytes in the host white matter were of human origin. FIG. 3H shows that the overall density of transferrin-defined oligodendrocytes, whether of mouse or human origin, was relatively preserved at all time points. FIGS. 3I-3K show that, by 46-wks, adult-transplanted hGPCs are admixed with murine cells in the largely remyelinated corpus callosum (FIG. 3I). FIG. 3J shows that, by this point, most myelinating oligodendrocytes in the cuprizone-demyelinated callosal were of human donor origin (human nuclear antigen; MBP; DAPI), just as many of the resident human cells had differentiated as TF-defined oligodendrocytes (FIG. 3K, human nuclear antigen; transferrin). Scale: FIG. 3I: 100 μm; FIG. 3J: 50 μm. FIG. 3K, 10 μm

FIGS. 4A-4D show hGPCs transcriptional networks augur compensatory remyelination after demyelination. Human glial chimeras were maintained on either a cuprizone (CZN)-containing or control diet from 12-24 weeks of age. 12 weeks later, at 36 weeks, the mice were killed and their resident hGPCs isolated via CD140a-based FACS, which were then subjected to RNA-Seq (n=6). FIG. 4A shows principle component analysis revealed tight clustering of hGPCs separated from post-CZN samples. FIG. 4B shows isolated hGPCs were enriched for genes indicative of an oligodendrocytic fate; gene expression representative of other lineages was minimal. FIG. 4C shows a network constructed from differentially expressed genes (circles) between post-CZN and CTR hGPCs (adjusted p<0.05); significantly associated gene ontology (GO) annotations (triangles) identified those pertinent and functionally related genes (gene nodes) that were differentially active in CZN-mobilized hGPCs. Gene node size was determined by the degree of connectivity, while annotation node sizes scaled with their adjusted p-values. Unsupervised modularity detection identified four modules (M) of closely related genes and annotations, for which a summary of annotations is provided along with the percentage of total gene connectivity for each module. Complete network information is offered in Table 5. FIG. 4D is a heatmap representation of genes identified in the previous GO network, organized by functional category and module membership (M).

FIGS. 5A-5B show enrichment of remyelination-associated pathways in cuprizone-exposed human GPCs. In FIG. 5A, the significantly enriched functional categories highlighted in FIG. 4C are organized by color-defined modules. Enrichment was determined via Fisher's Exact Test in Ingenuity Pathway Analysis. FIG. 5B shows genes differentially expressed by CZN-exposed hGPCs relative to controls, that contributed to these differentially-enriched pathways. Genes upregulated after cuprizone exposure and those down-regulated are shown. Activation Z-Scores are also provided for those pathways for which collective gene expression implies activation or inhibition, following CZN exposure in post-CZN vs. CTR hGPCs. Activation Z-Scores >1 were deemed significant.

### DETAILED DESCRIPTION

The disclosure herein relates generally to methods of treating a human subject having a condition mediated by a deficiency in myelin and methods of increasing oligodendrocyte production from human glial progenitor cells. These methods involve selecting a human subject having a condition mediated by a deficiency in myelin or providing a population of human glial progenitor cells and administering to the subject or the population of human glial progenitor cells one or modulators of a cell signaling pathway selected from the group consisting of Notch signaling, cAMP signaling, CIP2A signaling, RXRA signaling, TCF7L2 signaling, and combinations thereof, under conditions effective to treat the condition or increase oligodendrocyte production.

Exemplary genes, and their proteins encoded therefrom, involved in the cell signaling pathways described above include, without limitation, those shown in Table 1 and Table 2 below.

TABLE 1
Genes Enriched in Cuprizone treated hGPCS Compared to Controls

| | | | |
|---|---|---|---|
| EXT1 | Exostosin-1 | BHLHE41 | Basic Helix-Loop- |
| | | | Helix Family |
| | | | Member E41 |
| NCAM1 | neural cell adhesion | CARMIL1 | Capping Protein |
| | molecule 1 | | Regulator And Myosin 1 |
| | | | Linker 1 |
| NRP2 | Neuropilin 2 | CCL16 | Chemokine (C-C |
| | | | motif) ligand 16 |
| ROBO1 | roundabout guidance | CD22 | CD22 Molecule |
| | receptor 1 | | |
| ALDH1A2 | Aldehyde Dehydrogenase | CDCP1 | CUB Domain |
| | 1 Family Member A2 | | Containing Protein 1 |
| ANLN | Anilin | CDH5 | Cadherin 5 |
| CASR | calcium sensing receptor | CELSR1 | Cadherin EGF LAG |
| | | | Seven- Pass G-Type |
| | | | Receptor 1 |
| CERS2 | ceramide synthase 2 | CFH | Complement Factor H |
| CLDN11 | Claudin-11 | CHN2 | Chimerin 2 |
| GSN | gel solin | CHRNA7 | Cholinergic Receptor |
| | | | Nicotinic Alpha 7 |
| | | | Subunit |
| MOBP | Myelin-Associated | CLEC5A | C-Type Lectin |
| | Oligodendrocyte | | Domain Containing |
| | Basic Protein | | 5A |
| NKAIN2 | Sodium/Potassium | CMTM5 | CKLF-like MARVEL |
| | Transporting | | transmembrane domain- |
| | ATPase Interacting 2 | | containing protein 5 |
| PLEKHA1 | Pleckstrin Homology | CNTN2 | Contactin-2 |
| | Domain Containing A1 | | |
| PRRG1 | Proline-Rich Gla (G- | COL4A3 | Collagen Type IV |
| | Carboxyglutamic | | Alpha 3 Chain |
| | Acid) Polypeptide 1 | | |
| RAP1A | Ras-related protein Rap-1A | COL7A1 | Collagen Type VII |
| | | | Alpha 1 Chain |
| RNF13 | Ring Finger Protein 13 | CORO1B | coronin, actin |
| | | | binding protein 1B |
| SMAD7 | Mothers against | CSK | C-Terminal Src Kinase |
| | decapentaplegic homolog 7 | | |
| ST18 | suppression of | CYP2J2 | Cytochrome P450 Family |
| | tumorigenicity 18 | | 2 Subfamily J Member 2 |
| STRN | striatin | DACT2 | Dishevelled Binding |
| | | | Antagonist Of Beta |
| | | | Catenin 2 |
| BCKDK | Branched Chain | DBH | Dopamine |
| | Ketoacid | | Beta- |
| | Dehydrogenase Kinase | | Hydroxylase |
| DOCK7 | dedicator of cytokinesis 7 | DBN1 | Drebrin 1 |
| KIF14 | Kinesin Family Member 14 | DCN | Decorin |
| L1CAM | L1 Cell Adhesion Molecule | DOCK5 | dedicator of cytokinesis 5 |
| NFKBIA | NFKB inhibitor alpha | DRD2 | Dopamine Receptor D2 |
| RARG | Retinoic Acid | DTL | Denticleless E3 |
| | Receptor Gamma | | Ubiquitin Protein |
| | | | Ligase Homolog |
| TG | Thyroglobulin | EDIL3 | EGF Like Repeats |
| | | | And Discoidin |
| | | | Domains 3 |
| UGCG | UDP-Glucose Ceramide | EDN3 | Endothelin 3 |
| | Glucosyltransferase | | |
| ADORA2B | Adenosine A2b Receptor | EFNA1 | Ephrin A1 |
| ANOS1 | Anosmin 1 | ELF3 | E74 Like ETS |
| | | | Transcription Factor 3 |
| ARAP1 | ArfGAP With RhoGAP | FLT4 | Fms Related |
| | Domain | | Tyrosine Kinase 4 |
| ATG16L1 | Autophagy Related 16 Like 1 | FOXP3 | forkhead box P3 |
| FZD4 | Frizzled Class Receptor 4 | FURIN | Paired Basic Amino |
| | | | Acid Cleaving Enzyme |
| GIT1 | ARF GTPase- | GJB1 | gap junction beta 1 |
| | activating protein | | |
| GPR37 | G Protein-Coupled | HDAC9 | Histone Deacetylase 9 |
| | Receptor 37 | | |
| HNF4A | Hepatocyte Nuclear Factor | HOXB1 | Homeobox B1 |
| | 4 Alpha | | |
| HRH4 | Histamine H4 Receptor | IGSF8 | Immunoglobulin |
| | | | Superfamily Member 8 |
| IKZF1 | IKAROS Family Zinc Finger 1 | IL16 | Interleukin 16 |
| ITGAX | Integrin Subunit Alpha X | KIF1C | Kinesin Family Member 1C |
| LGR6 | Leucine Rich Repeat | LIPE | Lipase E |
| | Containing G Protein- | | |
| | Coupled Receptor 6 | | |
| LPAR1 | Lysophosphatidic | LTB4R2 | Leukotriene B4 Receptor 2 |
| | Acid Receptor 1 | | |
| MAP2K6 | Mitogen-Activated | MIXL1 | Mix Paired-Like |
| | Protein Kinase Kinase 6 | | Homeobox |
| MOG | myelin oligodendrocyte | MYO18A | Myosin XVIIIA |
| | glycoprotein | | |
| MYO1E | Myosin 1E | MYOCD | Myocardin |
| NCKAP1L | NCK Associated Protein 1 | NEO1 | Neogenin 1 |
| | Like | | |
| NLRP3 | NLR Family Pyrin | NOTCH3 | Neurogenic locus |
| | Domain Containing 3 | | notch homolog protein 3 |
| NOTCH4 | Neurogenic locus | NOX5 | NADPH Oxidase 5 |
| | notch homolog protein 4 | | |
| NR1I2 | Nuclear Receptor Subfamily | ONECUT2 | One Cut Homeobox 2 |
| | 1 Group I Member 2 | | |
| OPRM1 | Opioid Receptor Mu 1 | P4HA2 | Prolyl 4- |
| | | | hydroxylase |
| | | | subunit alpha-2 |
| PAX5 | Paired Box 5 | PARVG | Parvin Gamma |
| PEBP1 | Phosphatidylethanolamine | PCSK6 | Proprotein |
| | Binding Protein 1 | | Convertase |
| | | | Subtilisin/Kexin Type 6 |
| PLXNA3 | Plexin A3 | PLD1 | Phospholipase D1 |
| PTPRH | Protein Tyrosine | PRUNE | Prune |
| | Phosphatase, Receptor | | Exopolyphosphatase 1 |
| | Type H | | |
| RAPGEF3 | Rap guanine | RALBP1 | Ral-binding protein 1 |
| | nucleotide exchange | | |
| | factor 3 | | |
| SCN1B | Sodium Voltage- | RUVBL2 | RuvB Like AAA ATPase |
| | Gated Channel Beta | | 2 |
| | Subunit 1 | | |
| SEMA4C | Semaphorin 4C | SCN5A | Sodium Voltage-Gated |
| | | | Channel Alpha Subunit 5 |
| SH3BP1 | SH3 Domain Binding Protein 1 | SFTPA1 | Surfactant Protein A1 |
| SP100 | SP100 Nuclear Antigen | SIGLEC8 | Sialic acid-binding Ig- |
| | | | like lectin 8 |
| ST3GAL3 | beta-gal actoside-alpha- | SPN | Sialophorin |
| | 2,3-sialyltransferase-III | | |
| TMF1 | TATA Element | TFAP4 | Transcription Factor AP-4 |
| | Modulatory Factor 1 | | |
| TRPM8 | Transient receptor | TGFB1 | Transforming |
| | potential cation channel | | growth factor beta 1 |
| | subfamily M member 8 | | |
| VPS18 | CORVET/HOPS Core | TNFRSF11A | TNF Receptor |
| | Subunit | | Superfamily Member 11a |
| ZNF580 | Zinc Finger Protein 580 | TSC22D3 | TSC22 Domain |
| | | | Family Member 3 |
| CAB39 | Calcium-binding protein 39 | WASF1 | wiskott-Aldrich |
| | | | syndrome protein family |
| | | | member 1 |
| CYP17A | cytochrome P450c17a1 | XCR1 | X-C Motif Chemokine |
| | | | Receptor 1 |
| TUBA4A | Tubulin Alpha 4a | CDH5 | Cadherin 5 |
| AKR1C1 | Aldo-Keto Reductase | SLC22A18 | Solute Carrier Family |
| | Family 1 Member C1 | | 22 Member 18 |
| LPL | Lipoprotein Lipase | KMT2C | Lysine |
| | | | Methyltransferase 2C |
| SLCO1A2 | Solute Carrier Organic | LPCAT3 | Lyso- |
| | Anion Transporter Family | | phosphatidylcholine |
| | Member 1A2 | | Acyltransferase 3 |
| SLC16A2 | Solute Carrier Family | STRA6 | Stimulated By |
| | 16 Member 2 | | Retinoic Acid 6 |
| MON1A | MON1 Homolog A, | FXN | Frataxin |
| | Secretory Trafficking | | |
| | Associated | | |
| ANK1 | Ankyrin 1 | NDFIP1 | Nedd4 Family |
| | | | Interacting Protein 1 |
| CHRNA2 | Nicotinic acetylcholine | ADCY7 | Adenylate Cyclase 7 |
| | receptor alpha 2 | | |
| | subunit | | |
| GABRR2 | Gamma-Aminobutyric | DAAM2 | Dishevelled |
| | Acid Type A Receptor | | Associated Activator |
| | Rho2 Subunit | | Of Morphogenesis 2 |
| NBPF8 | neuroblastoma breakpoint | GTF2IRD1 | general transcription |
| | family, member 8 | | factor II I repeat |
| | | | domain- containing 1 |
| PCLO | Piccolo | NEDD8 | Neural Precursor Cell |
| | Presynaptic | | Expressed, |
| | Cytomatrix | | Developmentally |
| | Protein | | Down- Regulated 8 |
| PITPNM1 | Phosphatidylinositol | PCSK1 | Proprotein |
| | Transfer Protein Membrane | | Convertase |
| | Associated 1 | | Subtilisin/Kexin Type 1 |
| RIMS3 | Regulating Synaptic | RBFOX1 | RNA Binding |
| | Membrane Exocytosis 3 | | Fox-1 Homolog 1 |
| AVPR1A | Arginine Vasopressin | SCN4A | Sodium Voltage-Gated |
| | Receptor 1A | | Channel Alpha Subunit 4 |
| LAIR1 | Leukocyte Associated | F2RL3 | F2R Like Thrombin |
| | Immunoglobulin | | Or Trypsin Receptor 3 |
| | Like Receptor 1 | | |
| TRPC3 | transient receptor protein 3 | OR51E2 | Olfactory Receptor |
| | | | Family 51 Subfamily E |
| | | | Member 2 |
| ADCY2 | Adenylate Cyclase 2 | WFS1 | Wolframin ER |
| | | | Transmembrane |
| | | | Glycoprotein |
| CAMK4 | Calcium/Calmodulin | GDE1 | Glycerophosphodiester |
| | Dependent Protein Kinase | | Phosphodiesterase 1 |
| | IV | | |
| PKIG | CAMP-Dependent | FGFRL1 | Fibroblast Growth |
| | Protein Kinase Inhibitor | | Factor Receptor Like 1 |
| | Gamma | | |
| PDK3 | Pyruvate | HCN1 | hyperpolarization |
| | Dehydrogenase Kinase 3 | | activated cyclic |
| | | | nucleotide gated |
| | | | potassium channel 1 |
| FEZ1 | Fasciculation And | ZIC4 | Zic Family Member 4 |
| | Elongation Protein Zeta 1 | | |
| AK8 | Adenylate Kinase 8 | HS2ST1 | Heparan Sulfate 2- |
| | | | O- Sulfotransferase 1 |
| MECOM | ecotropic virus | HPCAL4 | Hippocalcin Like 4 |
| | integration site 1 protein | | |
| | homolog | | |
| NME5 | NME/NM23 Family | PSPN | Persephin |
| | Member 5 | | |
| PPT1 | Palmitoyl- | FMN1 | Formin 1 |
| | Protein | | |
| | Thioesterase 1 | | |
| SMG9 | Nonsense Mediated | TUBB1 | Tubulin Beta 1 Class VI |
| | MRNA Decay Factor | | |
| PDGFC | Platelet Derived | TMEM135 | Transmembrane |
| | Growth Factor C | | Protein 135 |
| CDH5 | Cadherin 5 | ACAT1 | Acetyl-CoA |
| | | | Acetyltransferase 1 |
| GJA5 | Gap Junction Protein Alpha 5 | GNPDA1 | Glucosamine-6- |
| | | | Phosphate Deaminase 1 |
| PLCD3 | Phospholipase C Delta 3 | TDP1 | Tyrosyl-DNA |
| | | | phosphodiesterase 1 |
| SLCO1C1 | Solute Carrier Organic | LETM1 | Leucine Zipper And EF- |
| | Anion Transporter Family | | Hand Containing |
| | Member 1C1 | | Transmembrane Protein 1 |
| DGAT2 | Diacylglycerol O- | IL4R | Interleukin 4 Receptor |
| | Acyltransferase 2 | | |
| ACBD3 | Acyl-CoA Binding | | |
| | Domain Containing 3 | | |
| SLC13A5 | Solute Carrier Family | | |
| | 13 Member 5 | | |
| GNPNAT1 | Glucosamine-Phosphate N- | | |
| | Acetyltransferase 1 | | |
| KCNMB1 | potassium calcium- | | |
| | activated channel | | |
| | subfamily M regulatory | | |
| | beta subunit 1 | | |

TABLE 2
Genes Downregulated in Curpizone treated hGPCS Compared to Controls

| | | | |
|---|---|---|---|
| JAG1 | Jagged 1 | TSPAN15 | Tetraspanin 15 |
| AHCY | Adenosylhomocysteinase | CAMK4 | Calcium/Calmodulin |
| | | | Dependent Protein Kinase IV |
| CAMSAP3 | Calmodulin Regulated | CIP2A | Cell Proliferation |
| | Spectrin Associated | | Regulating Inhibitor Of |
| | Protein Family Member 3 | | Protein Phosphatase 2A |
| MINK1 | Misshapen Like Kinase 1 | TAR5 | Trace Amine Receptor 5 |
| TNFRSF25 | TNF Receptor | PAK3 | P21 (RAC1) Activated |
| | Superfamily Member 25 | | Kinase 3 |
| SPRY1 | Sprouty RTK | TRIM21 | Tripartite Motif |
| | Signaling Antagonist 1 | | Containing 21 |

As used herein, the term “glial cells” refers to a population of non-neuronal cells that provide support and nutrition, maintain homeostasis, either form myelin or promote myelination, and participate in signal transmission in the nervous system. “Glial cells” as used herein encompasses fully differentiated cells of the glial lineage, such as oligodendrocytes or astrocytes, and well as glial progenitor cells. Glia progenitor cells are cells having the potential to differentiate into cells of the glial lineage such as oligodendrocytes and astrocytes.

As used herein, “treating” or “treatment” refers to any indication of success in amelioration of an injury, pathology, or condition, including any objective or subjective parameter such as abatement; remission; diminishing of symptoms or making the injury, pathology, or condition more tolerable to the patient; slowing the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation. “Treating” includes the administration of glial progenitor cells to prevent or delay, to alleviate, or to arrest or inhibit development of the symptoms or conditions associated with the disease, condition or disorder. “Therapeutic effect” refers to the reduction, elimination, or prevention of the disease, symptoms of the disease, or side effects of a disease, condition or disorder in the subject. Treatment may be prophylactic (to prevent or delay the onset or worsening of the disease, condition or disorder, or to prevent the manifestation of clinical or subclinical symptoms thereof) or therapeutic suppression or alleviation of symptoms after the manifestation of the disease, condition or disorder.

Suitable subjects for treatment in accordance with the methods described herein include any human subject having a condition mediated by a deficiency in myelin.

In one embodiment, the condition mediated by a deficiency in myelin is selected from the group consisting of pediatric leukodystrophies, the lysosomal storage diseases, congenital dysmyelination, cerebral palsy, inflammatory demyelination, post-infectious and post-vaccinial leukoencephalitis, radiation- or chemotherapy induced demyelination, and vascular demyelination.

In another embodiment, the condition mediated by a deficiency in myelin is selected from the group consisting of Pelizaeus-Merzbacher Disease, Tay-Sach Disease, Sandhoff's gangliosidoses, Krabbe's disease, metachromatic leukodystrophy, mucopolysaccharidoses, Niemann-Pick A disease, adrenoleukodystrophy, Canavan's disease, Vanishing White Matter Disease, and Alexander Disease.

In a further embodiment, the condition mediated by a deficiency in myelin is selected from the group consisting of multiple sclerosis, neuromyelitis optica, transverse myelitis, optic neuritis, subcortical stroke, diabetic leukoencephalopathy, hypertensive leukoencephalopathy, age-related white matter disease, spinal cord injury, radiation- or chemotherapy induced demyelination, post-infectious and post-vaccinial leukoencephalitis, periventricular leukomalacia, and cerebral palsy.

The one or more modulators for use in the methods described herein can be, without limitation, a peptide, nucleic acid molecule, or small molecule compound. The modulator may be, for example, a naturally occurring, semi-synthetic, or synthetic agent. For example, the modulator may be a drug that targets a specific function of one or more genes. In certain embodiments, the one or more modulators may be an antagonist or an agonist.

The modulators of the present application can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The modulators of the present application may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these modulators may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained. Preferred compositions according to the present application are prepared so that an oral dosage unit contains between about 1 and 250 mg of active compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These modulators may also be administered parenterally. Solutions or suspensions of these modulators can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The modulators of the present application may also be administered directly to the airways in the form of an aerosol. For use as aerosols, the compounds of the present application in solution or suspension may be packaged in a pressurized aerosol container together with suitable propellants, for example, hydrocarbon propellants like propane, butane, or isobutane with conventional adjuvants. The materials of the present application also may be administered in a non-pressurized form such as in a nebulizer or atomizer.

If modulation is to be achieved at the DNA level, this may be done using gene therapy to knock-out or disrupt the target gene. As used herein, a “knock-out” can be a gene knockdown or the gene can be knocked out by a mutation such as, a point mutation, an insertion, a deletion, a frameshift, or a missense mutation by techniques known in the art, including, but not limited to, retroviral gene transfer.

In one embodiment, the one or more modulators may repress the expression of one or more of the genes described herein via a zinc finger nuclease. Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome (Urnov et al., “Genome Editing with Engineered Zinc Finger Nucleases,” Nat. Rev. Genet. 11:636-646 (2010), which is hereby incorporated by reference in its entirety). By taking advantage of endogenous DNA repair machinery, these reagents can be used to precisely alter the genomes of higher organisms.

The one or more modulators may also be a meganuclease and TAL effector nuclease (TALENs, Cellectis Bioresearch) (Joung & Sander, “TALENs: A Widely Applicable Technology for Targeted Genome Editing,” Nat. Rev. Mol. Cell Biol. 14:49-55 (2013), which is hereby incorporated by reference in its entirety). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes).

In another embodiment, the one or more modulators is a CRISPR-Cas9 guided nuclease (Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121):819-23 (2013); and Gaj et al., “ZFN, TALEN, and CRISPR/Cas-based Methods for Genome Engineering,” Cell 31(7):397-405 (2013), which are hereby incorporated by reference in their entirety). Like the TALENs and ZFNs, CRISPR-Cas9 interference is a genetic technique which allows for sequence-specific control of gene expression in prokaryotic and eukaryotic cells by guided nuclease double-stranded DNA cleavage. It is based on the bacterial immune system-derived CRISPR (clustered regularly interspaced palindromic repeats) pathway.

Modulation of the one or more cell signaling pathways described herein can also be carried out using antisense oligonucleotides (ASO). Suitable therapeutic ASOs for inhibition of one or more of the cell signaling pathways described herein include, without limitation, antisense RNAs, DNAs, RNA/DNA hybrids (e.g., gapmer), and chemical analogues thereof, e.g., morpholinos, peptide nucleic acid oligomer, ASOs comprised of locked nucleic acids. With the exception of RNA oligomers, PNAs, and morpholinos, all other antisense oligomers act in eukaryotic cells through the mechanism of RNase H-mediated target cleavage. PNAs and morpholinos bind complementary DNA and RNA targets with high affinity and specificity, and thus act through a simple steric blockade of the RNA translational machinery, and appear to be completely resistant to nuclease attack.

An “antisense oligomer” refers to an antisense molecule or anti-gene agent that comprises an oligomer of at least about 10 nucleotides in length. In some embodiments, an antisense oligomer comprises at least 15, 18, 20, 25, 30, 35, 40, or 50 nucleotides. Antisense approaches involve the design of oligonucleotides (either DNA, RNA, DNA/RNA, or chemically modified derivatives thereof) that are complementary to an RNA encoded by polynucleotide sequences of the genes identified herein. Antisense RNA may be introduced into a cell to inhibit translation or activity of a complementary mRNA by base pairing to it and physically obstructing its translation or its activity. This effect is therefore stoichiometric. Absolute complementarity, although preferred, is not required. A sequence “complementary” to a portion of an RNA, as referred to herein, means a sequence having sufficient complementarity to be able to hybridize with the RNA, forming a stable duplex. In the case of double stranded antisense polynucleotide sequences, a single strand of the duplex DNA may thus be tested, or triplex formation may be assayed. The ability to hybridize will depend on both the degree of complementarity and the length of the antisense polynucleotide sequence. Generally, the longer the hybridizing polynucleotide sequence, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, the one or more modulators is an antisense oligonucleotide that specifically binds to and inhibits the functional expression of one or more genes involved in the cell signaling pathways described herein. For example, common modifications to an ASO to increase duplex stability include the incorporation of 5-methyl-dC, 2-amino-dA, locked nucleic acid, and/or peptide nucleic acid bases. Common modifications to enhance nuclease resistance include conversion of the normal phosphodiester linkages to phosphorothioate or phosphorodithioate linkages, or use of propyne analog bases, 2′-O-Methyl or 2′-O-Methyloxyethyl RNA bases.

RNA interference (RNAi) using small interfering RNA (siRNA) is another form of post-transcriptional gene silencing that can be utilized for modulating one or more cell signaling pathways in a subject as described herein.

Accordingly, in one embodiment, the one or more modulators is an siRNA. siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule. siRNA molecules are typically designed to target a region of the mRNA target approximately 50-100 nucleotides downstream from the start codon. The siRNAs of the present application can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion. Upon introduction into a cell, the siRNA complex triggers the endogenous RNAi pathway, resulting in the cleavage and degradation of the target mRNA molecule. Various improvements of siRNA compositions, such as the incorporation of modified nucleosides or motifs into one or both strands of the siRNA molecule to enhance stability, specificity, and efficacy, have been described and are suitable for use in accordance with this aspect of the present application (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety).

In another embodiment, the one or more modulators comprises endoribonuclease-prepared siRNAs (esiRNA), which comprise a mixture of siRNA oligonucleotides formed from the cleavage of long double stranded RNA with an endoribonuclease (e.g., RNase III or dicer). Digestion of synthetic long double stranded RNA produces short overlapping fragments of siRNAs with a length of between 18-25 bases that all target the same mRNA sequence. The complex mixture of many different siRNAs all targeting the same mRNA sequence leads to increased silencing efficacy. The use of esiRNA technology to target long non-coding RNA has been described in the art (Theis et al., “Targeting Human Long Noncoding Transcripts by Endoribonuclease-Prepared siRNAs,” J. Biomol. Screen 20(8):1018-1026 (2015), which is hereby incorporated by reference in its entirety).

The one or more modulators may also be a short or small hairpin RNA. Short or small hairpin RNA molecules are similar to siRNA molecules in function, but comprise longer RNA sequences that make a tight hairpin turn. shRNA is cleaved by cellular machinery into siRNA and gene expression is silenced via the cellular RNA interference pathway.

Nucleic acid aptamers that specifically bind to one or more of the genes involved in the cell signaling pathways described herein are also useful in the methods of the present application. Nucleic acid aptamers are single-stranded, partially single-stranded, partially double-stranded, or double-stranded nucleotide sequences, advantageously a replicatable nucleotide sequence, capable of specifically recognizing a selected non-oligonucleotide molecule or group of molecules by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides, and nucleotides comprising backbone modifications, branchpoints, and non-nucleotide residues, groups, or bridges. Nucleic acid aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence, promoter, or primer-annealing sequence needed to amplify, transcribe, or replicate all or part of the aptamer molecule or sequence.

In the embodiments described supra, the one or more modulators may be packaged in a suitable delivery vehicle or carrier for delivery to the subject. Suitable delivery vehicles include, but are not limited to viruses, virus-like particles, bacteria, bacteriophages, biodegradable microspheres, microparticles, nanoparticles, exosomes, liposomes, collagen minipellets, and cochleates. These and other biological gene delivery vehicles are well known to those of skill in the art (see e.g., Seow and Wood, “Biological Gene Delivery Vehicles: Beyond Viral Vectors,” Mol. Therapy 17(5):767-777(2009), which is hereby incorporated by reference in its entirety).

In one embodiment, the modulator is packaged into a therapeutic expression vector to facilitate delivery. Suitable expression vectors are well known in the art and include, without limitation, viral vectors such as adenovirus vectors, adeno-associated virus vectors, retrovirus vectors, lentivirus vectors, or herpes virus vectors.

The viral vectors or other suitable expression vectors comprise sequences encoding the inhibitory nucleic acid molecule (e.g., siRNA, ASO, etc.) of the present application and any suitable promoter for expressing the inhibitory sequences. Suitable promoters include, for example, and without limitation, the U6 or HI RNA pol III promoter sequences and the cytomegalovirus promoter. Selection of other suitable promoters is within the skill in the art. The expression vectors may also comprise inducible or regulatable promoters for expression of the inhibitory nucleic acid molecules in a tissue or cell-specific manner.

Gene therapy vectors carrying the therapeutic inhibitory nucleic acid molecule are administered to a subject by, for example, intravenous injection, local administration (U.S. Pat. No. 5,328,470 to Nabel et al., which is hereby incorporated by reference in its entirety) or by stereotactic injection (see e.g., Chen et al. “Gene Therapy for Brain Tumors: Regression of Experimental Gliomas by Adenovirus Mediated Gene Transfer In Vivo,” Proc. Nat'l. Acad. Sci. USA 91:3054-3057 (1994), which is hereby incorporated by reference in its entirety). The pharmaceutical preparation of the therapeutic vector can include the therapeutic vector in an acceptable diluent, or can comprise a slow release matrix in which the therapeutic delivery vehicle is imbedded. Alternatively, where the complete therapeutic delivery vector can be produced intact from recombinant cells, e.g., retroviral vectors, the pharmaceutical preparation can include one or more cells which produce the therapeutic delivery system. Gene therapy vectors typically utilize constitutive regulatory elements which are responsive to endogenous transcriptions factors.

Another suitable approach for the delivery of the modulators of the present disclosure, involves the use of liposome delivery vehicles or nanoparticle delivery vehicles.

In one embodiment, the pharmaceutical composition or formulation containing an inhibitory nucleic acid molecule (e.g., siRNA molecule) is encapsulated in a lipid formulation to form a nucleic acid-lipid particle as described in Semple et al., “Rational Design of Cationic Lipids for siRNA Delivery,” Nature Biotech. 28:172-176 (2010) and WO2011/034798 to Bumcrot et al., WO2009/111658 to Bumcrot et al., and WO2010/105209 to Bumcrot et al., which are hereby incorporated by reference in their entirety. Other cationic lipid carriers suitable for the delivery of ASO include, without limitation, N-[1-(2,3-dioleoyloxy)propyl]-N,N,N-trimethylammonium chloride (DOTMA) and N-[1-(2,3-dioleoyloxy)propyl]-N,N,N-trimethylammonium methyl sulphate (DOTAP) (see Chan et al., “Antisense Oligonucleotides: From Design to Therapeutic Application,” Clin. Exp. Pharm. Physiol. 33: 533-540 (2006), which is hereby incorporated by reference in its entirety).

In another embodiment of the present application, the delivery vehicle is a nanoparticle. A variety of nanoparticle delivery vehicles are known in the art and are suitable for delivery of the modulators of the present application (see e.g., van Vlerken et al., “Multi-functional Polymeric Nanoparticles for Tumour-Targeted Drug Delivery,” Expert Opin. Drug Deliv. 3(2):205-216 (2006), which is hereby incorporated by reference in its entirety). Suitable nanoparticles include, without limitation, poly(beta-amino esters) (Sawicki et al., “Nanoparticle Delivery of Suicide DNA for Epithelial Ovarian Cancer Cell Therapy,” Adv. Exp. Med. Biol. 622:209-219 (2008), which is hereby incorporated by reference in its entirety), polyethylenimine-alt-poly(ethylene glycol) copolymers (Park et al., “Degradable Polyethylenimine-alt-Poly(ethylene glycol) Copolymers As Novel Gene Carriers,” J Control Release 105(3):367-80 (2005) and Park et al., “Intratumoral Administration of Anti-KITENIN shRNA-Loaded PEI-alt-PEG Nanoparticles Suppressed Colon Carcinoma Established Subcutaneously in Mice,” J Nanosci. Nanotechnology 10(5):3280-3 (2010), which are hereby incorporated by reference in their entirety), poly(d,l-lactide-coglycolide) (Chan et al., “Antisense Oligonucleotides: From Design to Therapeutic Application,” Clin. Exp. Pharm. Physiol. 33: 533-540 (2006), which is hereby incorporated by reference in its entirety), and liposome-entrapped siRNA nanoparticles (Kenny et al., “Novel Multifunctional Nanoparticle Mediates siRNA Tumor Delivery, Visualization and Therapeutic Tumor Reduction In Vivo,” J. Control Release 149(2):111-116 (2011), which is hereby incorporated by reference in its entirety). Other nanoparticle delivery vehicles suitable for use in the present application include microcapsule nanotube devices disclosed in U.S. Patent Publication No. 2010/0215724 to Prakash et al., which is hereby incorporated by reference in its entirety.

In another embodiment, the pharmaceutical composition is contained in a liposome delivery vehicle. The term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

Several advantages of liposomes include: their biocompatibility and biodegradability, incorporation of a wide range of water and lipid soluble drugs; and they afford protection to encapsulated drugs from metabolism and degradation. Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Methods for preparing liposomes include those disclosed in Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., which are hereby incorporated by reference in their entirety.

Exemplary modulators, the cell signaling pathways targeted by such modulators, and the resulting effect on gene expression are shown in Table 3 below.

TABLE 3

| | | | Pathway Genes | | cAMP | | | | |
|---|---|---|---|---|---|---|---|---|---|
| Drug | | Pathway Genes | Down- | | Mediated | CIP2A | Notch | RXRA | TCF7L2 |
| Name | Broad ID | Upregulated | regulated | Pathway | Signaling | Signaling | Signaling | Signaling | Signaling |
| Trichostatin | BRD-A19037878 | ACAA1, ADCY9, | DUSP1, | Notch | X | | X | | X |
| A | | ALOX5, CD24, | DUSP4, | | | | | | |
| | | CPD, CYP51A1, | ID4 | | | | | | |
| | | DHCR24, EPAS1, | | | | | | | |
| | | ERBB3, GSN, | | | | | | | |
| | | GNAI1, HES1, | | | | | | | |
| | | IDH1, JAG1, | | | | | | | |
| | | MAN1A1, ME1, | | | | | | | |
| | | NOTCH1, | | | | | | | |
| | | NOTCH3, NPC1, | | | | | | | |
| | | PAPSS1, PLAT, | | | | | | | |
| | | RAB31, SRD5A1, | | | | | | | |
| | | CAMK2N1, CAP1, | | | | | | | |
| | | CCND2, CD24, | | | | | | | |
| | | CPD, DOCK9, | | | | | | | |
| | | FGFR2, MAP7, | | | | | | | |
| | | PELI1, PPP1R16B, | | | | | | | |
| | | PRKAR2B, | | | | | | | |
| | | RAP1GAP, | | | | | | | |
| | | RPS6KA1 | | | | | | | |
| BRD- | BRD-K08438429 | AGTR1, CFB, | CCND1, | CIP2A | X | X | | | |
| K08438429 | | COL15A1, DCN, | DUSP6, | | | | | | |
| | | FAP, GNAI1, LXN, | PKIA, | | | | | | |
| | | PRKAR2B, | PKIG | | | | | | |
| | | PTGER2, SAT1, | | | | | | | |
| | | SERPINE2 | | | | | | | |
| Ichthynone | BRD-K28167849 | ADRB2, C4BPA, | PDE2A, | CIP2A | X | X | | | |
| | | CALML5, CRLF1, | RGS2, | | | | | | |
| | | CRYAB, GNAI1, | RGS4 | | | | | | |
| | | GPR183, HCAR3, | | | | | | | |
| | | LPAR1, LUM, | | | | | | | |
| | | P2RY14, PDLIM7, | | | | | | | |
| | | SRC | | | | | | | |
| Vorinostat | BRD-K81418486 | ADCY9, ADRB2, | RGS4 | Notch | X | | X | | |
| | | APLNR, CALM1, | | | | | | | |
| | | HES1, JAG1, | | | | | | | |
| | | JAG2, NOTCH1, | | | | | | | |
| | | NOTCH2, | | | | | | | |
| | | NOTCH3, | | | | | | | |
| | | PRKAR2B, | | | | | | | |
| | | RAP1GAP, | | | | | | | |
| | | RPS6KA1, | | | | | | | |
| | | CAMK2G, | | | | | | | |
| | | TBXA2R | | | | | | | |
| BRD- | BRD-K30523950 | ACSL1, | ID1, ID2, | TCF7L2 | X | | | | X |
| K30523950 | | ADORA2B, | PKIG, | | | | | | |
| | | ADRB2, APOD, | PPP3CA | | | | | | |
| | | ASPA, CAMK2N1, | | | | | | | |
| | | CCP110, DOCK9, | | | | | | | |
| | | ENPP2, ERBB3, | | | | | | | |
| | | FGFR2, FTH1, | | | | | | | |
| | | GNAS, HSPA2, | | | | | | | |
| | | IPO13, IRS2, | | | | | | | |
| | | MAN1A1, MOBP, | | | | | | | |
| | | PLP1, PPP1R16B, | | | | | | | |
| | | PRKAR1A, | | | | | | | |
| | | PTGER4 | | | | | | | |
| BJM-ctd2-9 | BRD-M86331534 | COL15A1, DCN, | GPNMB | Notch | | X | X | | |
| | | DSC2, GADD45A, | | | | | | | |
| | | GPR37, HES1, | | | | | | | |
| | | IER3, JAG1, | | | | | | | |
| | | NOTCH1, SAT1 | | | | | | | |
| BRD- | BRD-K51126483 | CFB, CRYAB, | C1QA, | CIP2A | | X | | X | |
| K51126483 | | DSC2, ECM1, | CCL8, | | | | | | |
| | | FABP1, GAS6, | DLK1, | | | | | | |
| | | GPX1, | E2F2 | | | | | | |
| | | HIST1H2BK, | | | | | | | |
| | | PLAUR, S100A8, | | | | | | | |
| | | SLC22A18, VCAN | | | | | | | |
| DO 897/99 | BRD-K17378184 | ALDH1A2, | C1QA, | CIP2A | | X | | X | |
| | | C4BPA, CCND1, | CXCL10, | | | | | | |
| | | CFB, CRABP2, | DLK1, | | | | | | |
| | | FLRT3, | E2F2, | | | | | | |
| | | HIST1H2BK, | MFAP5 | | | | | | |
| | | IGFBP6, LPL, | | | | | | | |
| | | LYZ, PLAUR, | | | | | | | |
| | | RET, SERPINE2, | | | | | | | |
| | | VCAN | | | | | | | |
| Pifithrin-a | BRD-K66874953 | ALDH1A2, | CCL8, | Notch | | | X | X | |
| | | ECM1, FSTL1, | MARCO | | | | | | |
| | | HBEGF, | | | | | | | |
| | | HES1IGFBP6, | | | | | | | |
| | | JAG1, LCN2, | | | | | | | |
| | | LPL, NOTCH2, | | | | | | | |
| | | NOTCH3, | | | | | | | |
| | | S100A8 | | | | | | | |
| CI 976 | BRD-K88544581 | ALDH1A1, | ABCC3, | RXRA | | | | X | X |
| | | CCND1, CD24, | AQP3, | | | | | | |
| | | COLEC12, | BMP4, | | | | | | |
| | | CRABP2, DDC, | CCL20, | | | | | | |
| | | EGFR, ENPP2, | ID2, | | | | | | |
| | | EPAS1, FA2H, | ID3, | | | | | | |
| | | FABP4, GCLC, | TGFB2, | | | | | | |
| | | GPX1, HBEGF, | DLK1, | | | | | | |
| | | JAG1, LCN2, LPL, | WEE1 | | | | | | |
| | | MAG7, MBP, | | | | | | | |
| | | MCAM, NPY, | | | | | | | |
| | | PDGFA, PMP22, | | | | | | | |
| | | QKI, S100A8, | | | | | | | |
| | | SLC6A8, WISP2 | | | | | | | |
| Rolipram | BRD-A34255068 | ALDH1A2, | APOA1, | RXRA | | | | X | X |
| | | CCND1, CD24, | CCL20, | | | | | | |
| | | CDK19, CREB3L2, | CXCL10, | | | | | | |
| | | DOCK9, ENPP4, | DLK1, | | | | | | |
| | | GCLC, IGF1, | ID2 | | | | | | |
| | | KAT2B, MAN1A1, | | | | | | | |
| | | NFASC, NKX2-1, | | | | | | | |
| | | OLIG2, PLP1, | | | | | | | |
| | | PMP22, QKI, | | | | | | | |
| | | S100A8, SH3GL3, | | | | | | | |
| | | SLC12A2, | | | | | | | |
| | | SLC27A2, SOX10 | | | | | | | |
| BRD- | BRD-K64245000 | ADORA2B, | CREM, | cAMP | X | | | | |
| K64245000 | | CHRM3, CNR1, | RGS2 | | | | | | |
| | | GNAS, HCAR3, | | | | | | | |
| | | PRKAR2B, | | | | | | | |
| | | VIPR1 | | | | | | | |
| NF 449 | BRD-K36324071 | ADRB2, HCAR3, | PKIA, | cAMP | X | | | | |
| | | P2RY13, P2RY14, | PKIG, | | | | | | |
| | | PTGDR, PTGER4 | PPP3CA | | | | | | |
| BRD- | BRD-A34751532 | ADCY7, ADCY9, | DUSP1 | cAMP | X | | | | |
| A34751532 | | GABBR2, HCAR3, | | | | | | | |
| | | PRKAR2B, | | | | | | | |
| | | PTGER2, PTGER3 | | | | | | | |
| BRD- | BRD-K63938928 | ADORA2B, | PPP3CA | cAMP | X | | | | |
| K63938928 | | ADRB2, GNAS, | | | | | | | |
| | | P2RY14, | | | | | | | |
| | | PTGER4, SRC, | | | | | | | |
| | | TBXA2R | | | | | | | |
| BRD- | BRD-K64402243 | ADCY7, ADRB2, | RGS2 | cAMP | X | | | | |
| K64402243 | | CALM1, HCAR3, | | | | | | | |
| | | P2RY14, | | | | | | | |
| | | PRKAR2B, | | | | | | | |
| | | TBXA2R | | | | | | | |
| 7b-cis | BRD-K61829047 | ADRB2, GNAI1, | DUSP6 | cAMP | X | | | | |
| | | HCAR3, LPAR1, | | | | | | | |
| | | P2RY13, P2RY14, | | | | | | | |
| | | PRKACB, | | | | | | | |
| | | PRKAR2B | | | | | | | |
| BRD- | BRD-A36318220 | ADORA2B, | | cAMP | X | | | | |
| A36318220 | | ADRB2, CALML5, | | | | | | | |
| | | HCAR3, P2RY14, | | | | | | | |
| | | RAP1GAP, SRC | | | | | | | |
| BRD- | BRD-K09549677 | ADCY9, ADRB2, | RGS2 | cAMP | X | | | | |
| K09549677 | | GNAS, LPAR1, | | | | | | | |
| | | PTGER4, | | | | | | | |
| | | RAP1GAP | | | | | | | |
| BRD- | BRD-K71430621 | ADRB2, GNAI1, | DUSP6, | cAMP | X | | | | |
| K71430621 | | HCAR3, LPAR1, | PKIG, | | | | | | |
| | | PTGER4 | RGS2 | | | | | | |
| BRD- | BRD-K74212935 | GNAI1, LPAR1, | DUSP4 | cAMP | X | | | | |
| K74212935 | | NPR3, PRKACB, | | | | | | | |
| | | PRKAR2B, | | | | | | | |
| | | PTGER4 | | | | | | | |
| BRD- | BRD-K93623754 | ADORA2B, | DUSP1, | cAMP | X | | | | |
| K93623754 | | ADRB2, LPAR1, | PPP3CA, | | | | | | |
| | | PRKACB, | RGS2 | | | | | | |
| | | RAP1GAP | | | | | | | |
| Bumetanide | BRD-K38197229 | ADCY7, | DUSP4, | cAMP | X | | | | |
| | | PRKAR2B, | DUSP6, | | | | | | |
| | | PTGER3 | PKIG, | | | | | | |
| | | | RGS4 | | | | | | |
| Chloroquine | BRD-A91699651 | ADRB2, LPAR1, | PKIG | cAMP | X | | | | |
| Diphosphate | | P2RY13, PTGER4, | | | | | | | |
| | | RAP1GAP, SRC | | | | | | | |
| Laudanosine | BRD-A24817035 | ADCY7, AGTR1, | DUSP1 | cAMP | X | | | | |
| (R, S) | | CREB1, PRKACB, | | | | | | | |
| | | PTGER4, | | | | | | | |
| | | RPS6KA1, SRC, | | | | | | | |
| | | STAT3 | | | | | | | |
| PD-184352 | BRD-K05104363 | ADCY9, ADRB2, | DUSP4, | cAMP | X | | | | |
| | | PRKAR2B, STAT3 | DUSP6, | | | | | | |
| | | | RGS2 | | | | | | |
| PRL-3 | BRD-K09907482 | GABBR2, GNAI1, | DUSP6 | cAMP | X | | | | |
| Inhibitor I | | LPAR1, PRKACB, | | | | | | | |
| | | RPS6KA1, SRC | | | | | | | |
| Troxipide | BRD-A68589262 | ADCY1, GNAS, | PPP3CA | cAMP | X | | | | |
| | | P2RY14, PRKACB, | | | | | | | |
| | | PRKAR2B, STAT3 | | | | | | | |
| BRD- | BRD-K44276885 | CFB, CRLF1, | ACTG2, | CIP2A | | X | | | |
| K44276885 | | DCN, LUM, LXN, | CCND1, | | | | | | |
| | | SNCA | ZDHHC11 | | | | | | |
| Arachidonyl | BRD-K07303502 | DCN, GADD45A, | ACTG2, | CIP2A | | X | | | |
| trifluoro- | | IER3, PLAUR | CCND1, | | | | | | |
| methyl | | | MYC, | | | | | | |
| ketone | | | SLC25A4 | | | | | | |
| BRD- | BRD-A25234499 | COL15A1, DCN, | ACTG2, | CIP2A | | X | | | |
| A25234499 | | LXN, SERPINE2 | GPNM, | | | | | | |
| | | | PDE2A, | | | | | | |
| | | | ZDHHC11 | | | | | | |
| BRD- | BRD-A69636825 | CRLF1, DCN, | ACTG2, | CIP2A | | X | | | |
| A69636825 | | LUM, SAT1, | GPNMB | | | | | | |
| | | SERPINE2, | | | | | | | |
| | | SLC22A4 | | | | | | | |
| BRD- | BRD-K34170797 | CRLF1, DSC2, | GPNMB, | CIP2A | | X | | | |
| K34170797 | | GPR37, SAT1, | MFAP5, | | | | | | |
| | | SNCA | RAB31 | | | | | | |
| BRD- | BRD-K46445327 | CFB, DCN, | ACTG2, | CIP2A | | X | | | |
| K46445327 | | HIST1H2BK, | SLC25A4 | | | | | | |
| | | LUM, SERPINE2, | | | | | | | |
| | | SNCA | | | | | | | |
| BRD- | BRD-K49807497 | COL15A1, DCN, | ACTG2, | CIP2A | | X | | | |
| K49807497 | | GPR37, SERPINE2 | GRSF1, | | | | | | |
| | | | MFAP5, | | | | | | |
| | | | RAB31 | | | | | | |
| BRD- | BRD-K68548958 | COL15A1, | CCND1, | CIP2A | | X | | | |
| K68548958 | | CRYAB, DCN, | MYC | | | | | | |
| | | GADD45A, | | | | | | | |
| | | HIST1H2BK, SAT1 | | | | | | | |
| BRD- | BRD-K71879957 | CFB, COL15A1, | E2F2, | CIP2A | | X | | | |
| K71879957 | | DCN, GPR183, | MYC | | | | | | |
| | | HIST1H2BK, LUM | | | | | | | |
| Calcipotriol | BRD-K56429665 | CFB, DCN, LUM, | CCND1, | CIP2A | | X | | | |
| | | LXN, NPTX1, | MFAP5 | | | | | | |
| | | SERPINE2 | | | | | | | |
| GANT 58 | BRD-K64451768 | DCN, DSC2, FAP, | PDE2A | CIP2A | | X | | | |
| | | LUM, LXN, SAT1, | | | | | | | |
| | | SNCA | | | | | | | |
| Lamivudine | BRD-K02992638 | CFB, DCN, | CCND1 | CIP2A | | X | | | |
| | | GPR183, LUM, | | | | | | | |
| | | LXN, SAT1, | | | | | | | |
| | | SERPINE2 | | | | | | | |
| Radicicol | BRD-A39996500 | CFB, CRYAB, | ACTG2, | CIP2A | | X | | | |
| | | GADD45A, | CCND1, | | | | | | |
| | | HIST1H2BK, IER3, | GPNMB, | | | | | | |
| | | LRP4, LXN, SAT1, | MYC | | | | | | |
| | | SERPINE2, | | | | | | | |
| | | SLC22A18 | | | | | | | |
| 71748 | BRD-A61858259 | C4BPA, COL15A1, | | CIP2A | | X | | | |
| | | DCN, HIST1H2BK, | | | | | | | |
| | | KIAA1324, LUM, | | | | | | | |
| | | LXN | | | | | | | |
| 10006734 | BRD-K07403598 | CRYAB, DCN, | ACTG2 | CIP2A | | X | | | |
| | | DSC2, LUM, LXN, | | | | | | | |
| | | SERPINE2, | | | | | | | |
| | | SLC12A8 | | | | | | | |
| BRD- | BRD-A36630025 | CFB, GADD45A, | ACTG2, | CIP2A | | X | | | |
| A36630025 | | GPR183, IER3 | CCND1, | | | | | | |
| | | | E2F2 | | | | | | |
| BRD- | BRD-A41250203 | GADD45, | ACTG2, | CIP2A | | X | | | |
| A41250203 | | HIST1H2BK, | CCND1, | | | | | | |
| | | LRP4, SAT1 | MYC | | | | | | |
| BRD- | BRD-K00317371 | DCN, GADD45A, | MYC | CIP2A | | X | | | |
| K00317371 | | IER3, LUM, | | | | | | | |
| | | PLAUR, SAT1 | | | | | | | |
| BRD- | BRD-K13810148 | CRLF1, SAT1, | CCND1, | CIP2A | | X | | | |
| K13810148 | | SERPINE2, | GPNMB, | | | | | | |
| | | TUBA4A | MYC | | | | | | |
| BRD- | BRD-K41429297 | DCN, SAT1, | ACTG2, | CIP2A | | X | | | |
| K41429297 | | SERPINE2, | GPNMB, | | | | | | |
| | | TUBA4A | MFAP5 | | | | | | |
| BRD- | BRD-K49010888 | CFB, DCN, IER3, | CCND1 | CIP2A | | X | | | |
| K49010888 | | LXN, SAT1, | | | | | | | |
| | | SLC22A18 | | | | | | | |
| BRD- | BRD-K50214219 | DCN, DSC2, | | CIP2A | | X | | | |
| K50214219 | | GPR37, | | | | | | | |
| | | HIST1H2BK, | | | | | | | |
| | | LUM, RHOC, | | | | | | | |
| | | SAT1 | | | | | | | |
| BRD- | BRD-K99135512 | CFB, DCN, DSC2, | E2F2, | CIP2A | | X | | | |
| K99135512 | | HIST1H2BK, | MFAP5 | | | | | | |
| | | LUM, SERPINE2 | | | | | | | |
| COT-10b | BRD-K18190982 | DCN, GADD45A, | CCND1, | CIP2A | | X | | | |
| | | IER3, LUM, | MYC, | | | | | | |
| | | PDGFRB, SAT1 | SLC25A4 | | | | | | |
| Estriol | BRD-A84205515 | DCN, FAP, LUM, | E2F2 | CIP2A | | X | | | |
| Methyl | | LXN, SAT1, | | | | | | | |
| Ether | | SERPINE2 | | | | | | | |
| GDC-0941 | BRD-K52911425 | CFB, DCN, FAP, | | CIP2A | | X | | | |
| | | LUM, LXN, SAT1, | | | | | | | |
| | | SERPINE2, | | | | | | | |
| | | SLC22A18 | | | | | | | |
| HDAC6 | BRD-K69840642 | CRLF1, SAT1, | CCND1, | CIP2A | | X | | | |
| inhibitor | | SERPINE2, | GPNMB, | | | | | | |
| ISOX | | TUBA4A | MYC | | | | | | |
| KUC104141 | BRD-K43637719 | DCN, FAP, LUM, | | CIP2A | | X | | | |
| KUC104141N | | LXN, SAT1, | | | | | | | |
| | | SERPINE2, | | | | | | | |
| | | SLC22A18 | | | | | | | |
| Meclofenamate | BRD-K50398167 | CFB, COL15A1, | GRSF1 | CIP2A | | X | | | |
| Sodium | | DCN, GPR37, | | | | | | | |
| | | LUM, SNCA | | | | | | | |
| MLS- | BRD-K54070802 | CFB, DCN, LUM, | GPNMB, | CIP2A | | X | | | |
| 0315803 | | LXN, PLAUR, | MFAP5 | | | | | | |
| | | SAT1 | | | | | | | |
| NCGC00182837- | BRD-K40175949 | C4BPA, | GPNMB, | CIP2A | | X | | | |
| 01 | | HIST1H2BK, | MFAP5 | | | | | | |
| | | LRP4, LUM, | | | | | | | |
| | | TUBA4A | | | | | | | |
| NCGC00229626- | BRD-K81214387 | GADD45A, | GPNMB | CIP2A | | X | | | |
| 01 | | GPR183, GPR37, | | | | | | | |
| | | HIST1H2BK, | | | | | | | |
| | | LRP4, SAT1 | | | | | | | |
| NCGC00241438- | BRD-K82185908 | DCN, IER3, LXN, | CCND1, | CIP2A | | X | | | |
| 01 | | SAT1, SERPINE2 | PDE2A | | | | | | |
| Pipamperone | BRD-K26801045 | GPR37, | ACTG2, | CIP2A | | X | | | |
| | | HIST1H2BK, | CCND1, | | | | | | |
| | | SAT1, SLC12A8 | MFAP5 | | | | | | |
| PJ34 | BRD-K11853856 | CFB, COL15A1, | GRSF1 | CIP2A | | X | | | |
| Hydrochioride | | DCN, FAP, LUM, | | | | | | | |
| | | PDGFRB, PLAUR | | | | | | | |
| PX12 | BRD-A56592690 | CFB, DCN, EBI3, | ACTG2, | CIP2A | | X | | | |
| | | IER3 | GPNMB, | | | | | | |
| | | | MYC | | | | | | |
| SU6668 | BRD-K55966568 | CRYAB, DCN, | ACTG2, | CIP2A | | X | | | |
| | | ENO3, LUM, | RAB31 | | | | | | |
| | | SERPINE2 | | | | | | | |
| XMD11-85H | BRD-U22633929 | CFB, DCN, | E2F2, | CIP2A | | X | | | |
| | | HIST1H2BK, | PDK4 | | | | | | |
| | | LXN, SAT1 | | | | | | | |
| 5587525 | BRD-K74606027 | APH1A, HES1, | | Notch | | | X | | |
| | | JAG1, NOTCH1, | | | | | | | |
| | | NOTCH2 | | | | | | | |
| Acefylline | BRD-K44004064 | HES1, JAG1, | | Notch | | | X | | |
| | | NOTCH1, | | | | | | | |
| | | NOTCH2 | | | | | | | |
| BL-095 | BRD-K32610195 | HES1, NOTCH1, | | Notch | | | X | | |
| | | NOTCH2, | | | | | | | |
| | | NOTCH3 | | | | | | | |
| BMS 191011 | BRD-K95609758 | HES1, JAG1, | | Notch | | | X | | |
| | | NOTCH1, | | | | | | | |
| | | NOTCH2 | | | | | | | |
| BRD- | BRD-A21723284 | HES1, JAG1, | | Notch | | | X | | |
| A21723284 | | NOTCH1, | | | | | | | |
| | | NOTCH3 | | | | | | | |
| BRD- | BRD-K02275692 | HES1, JAG1, | | Notch | | | X | | |
| K02275692 | | NOTCH1, | | | | | | | |
| | | NOTCH2 | | | | | | | |
| BRD- | BRD-K11540476 | JAG1, NOTCH1, | KLF2 | Notch | | | X | | |
| K11540476 | | NOTCH2 | | | | | | | |
| BRD- | BRD-K11778076 | JAG1, NOTCH1, | | Notch | | | X | | |
| K11778076 | | NOTCH2, | | | | | | | |
| | | NOTCH3 | | | | | | | |
| BRD- | BRD-K15563106 | HES1, JAG1, | KLF2 | Notch | | | X | | |
| K15563106 | | NOTCH1 | | | | | | | |
| BRD- | BRD-K26573499 | HES1, JAG1, | | Notch | | | X | | |
| K26573499 | | NOTCH2, | | | | | | | |
| | | NOTCH3 | | | | | | | |
| BRD- | BRD-K28075147 | HES1, NOTCH1, | | Notch | | | X | | |
| K28075147 | | NOTCH2, | | | | | | | |
| | | NOTCH3 | | | | | | | |
| BRD- | BRD-K37618799 | HES1, JAG1, | KLF2 | Notch | | | X | | |
| K37618799 | | NOTCH3 | | | | | | | |
| BRD- | BRD-K38519699 | HES1, JAG2, | | Notch | | | X | | |
| K38519699 | | NOTCH1, | | | | | | | |
| | | NOTCH3 | | | | | | | |
| BRD- | BRD-K54708045 | JAG1, NOTCH1, | | Notch | | | X | | |
| K54708045 | | NOTCH2, | | | | | | | |
| | | NOTCH3 | | | | | | | |
| BRD- | BRD-K70947604 | JAG1, NOTCH1, | | Notch | | | X | | |
| K70947604 | | NOTCH2, | | | | | | | |
| | | NOTCH3 | | | | | | | |
| BRD- | BRD-K86108784 | HES1, NOTCH1, | KLF2 | Notch | | | X | | |
| K86108784 | | NOTCH3 | | | | | | | |
| BRD- | BRD-K93875449 | HES1, JAG1, | KLF2 | Notch | | | X | | |
| K93875449 | | NOTCH1 | | | | | | | |
| BRD- | BRD-K96041033 | HES1, JAG1, | | Notch | | | X | | |
| K96041033 | | NOTCH1, | | | | | | | |
| | | NOTCH2 | | | | | | | |
| IKK | BRD-K93023739 | HES1, JAG1, | | Notch | | | X | | |
| Inhibitor X | | NOTCH1, | | | | | | | |
| | | NOTCH3 | | | | | | | |
| L-750,667 | BRD-K28806945 | APH1A, HES1, | KLF2 | Notch | | | X | | |
| | | JAG1 | | | | | | | |
| L- | BRD-A58955223 | HES1, NOTCH1, | | Notch | | | X | | |
| sulforophane | | NOTCH2, | | | | | | | |
| | | NOTCH3 | | | | | | | |
| Metolazone | BRD-A61793559 | JAG1, NOTCH1, | KLF2 | Notch | | | X | | |
| | | NOTCH2 | | | | | | | |
| MLS- | BRD-K21640605 | JAG1, NOTCH1, | KLF2 | Notch | | | X | | |
| 0014097.0001 | | NOTCH3 | | | | | | | |
| Naloxone | BRD-K67511046 | HES1, NOTCH1, | | Notch | | | X | | |
| Hydrochioride | | NOTCH2, | | | | | | | |
| | | NOTCH3 | | | | | | | |
| NRB 04155 | BRD-K85266146 | HES1, JAG1, | | Notch | | | X | | |
| | | NOTCH2, | | | | | | | |
| | | NOTCH3 | | | | | | | |
| Prostaglandin | BRD-K04010869 | HES1, JAG1, | KLF2 | Notch | | | X | | |
| A1 | | NOTCH3 | | | | | | | |
| RG-13022 | BRD-K82688027 | HES1, JAG1, | KLF2 | Notch | | | X | | |
| | | NOTCH3 | | | | | | | |
| RS 16566 | BRD-K80725821 | HES1, JAG1, | | Notch | | | X | | |
| Hydrochioride | | JAG2, NOTCH1 | | | | | | | |
| STOCK2S- | BRD-K42568865 | APH1A, HES1, | | Notch | | | X | | |
| 25759 | | JAG1, NOTCH1 | | | | | | | |
| T5345967 | BRD-K38985961 | HES1, JAG1, | | Notch | | | X | | |
| | | NOTCH1, | | | | | | | |
| | | NOTCH2 | | | | | | | |
| Triacsin-c | BRD-K80527266 | HES1, JAG1, | KLF2 | Notch | | | X | | |
| | | NOTCH1 | | | | | | | |
| Tyrphostin | | HES1, JAG1, | | Notch | | | X | | |
| B44, (+) | | NOTCH1, | | | | | | | |
| Enantiomer | | NOTCH3 | | | | | | | |
| YM-155 | BRD-K76703230 | HES1, JAG1, | | Notch | | | X | | |
| | | NOTCH2, | | | | | | | |
| | | NOTCH3 | | | | | | | |
| BRD- | BRD-K00313977 | HES1, JAG1, | | Notch | | | X | | |
| K00313977 | | NOTCH1 | | | | | | | |
| BRD- | BRD-K43620258 | HES1, JAG1, | | Notch | | | X | | |
| K43620258 | | NOTCH1 | | | | | | | |
| BX-795 | BRD-K47983010 | HES1, JAG1 | KLF2 | Notch | | | X | | |
| Cefixime | BRD-K71059170 | JAG1, NOTCH3 | KLF2 | Notch | | | X | | |
| Cercosporin | BRD-A78360835 | HES1, JAG1, | | Notch | | | X | | |
| | | NOTCH1 | | | | | | | |
| Methylene | BRD-K16406336 | HES1, JAG1, | | Notch | | | X | | |
| Blue | | NOTCH1 | | | | | | | |
| Selamectin | BRD-A58564983 | HES1, JAG1, | | Notch | | | X | | |
| | | NOTCH1 | | | | | | | |
| VX-680 | BRD-K87947369 | JAG1, NOTCH1 | KLF2 | Notch | | | X | | |
| BRD- | BRD-K90610876 | ALDH1A1, | ABCC3, | RXRA | | | | X | |
| K90610876 | | ALDH1A2, | CYP3A5 | | | | | | |
| | | COLEC12, | | | | | | | |
| | | CRABP2, CTSB, | | | | | | | |
| | | GCLC, LCN2, | | | | | | | |
| | | LYZ, MMP12, | | | | | | | |
| | | S100A8 | | | | | | | |
| L5288-1MG | BRD-A19248578 | ALDH1A1, | ABCC3 | RXRA | | | | X | |
| | | ALDH1A2, | | | | | | | |
| | | CCNB1, COLEC12, | | | | | | | |
| | | FLRT3, LYZ, | | | | | | | |
| | | MMP12, S100A8, | | | | | | | |
| | | SLC6A8, VCAN | | | | | | | |
| 1,25- | BRD-K27316855 | ALDH1A2, | | RXRA | | | | X | |
| DIHYDROXY- | | CCNB1, CRABP2, | | | | | | | |
| VITAMIN | | CTSB, GPX1, | | | | | | | |
| D3 | | IGFBP6, IL1B, | | | | | | | |
| | | MAOB, PDGFA, | | | | | | | |
| | | SLC6A8, TPP1 | | | | | | | |
| 3- | BRD-K57954781 | ALDH1A2, | HMOX1 | RXRA | | | | X | |
| Deoxydenosine | | COLEC12, FSTL1, | | | | | | | |
| | | IGFBP6, LCN2, | | | | | | | |
| | | MAOB, MMP12, | | | | | | | |
| | | MMP2, PMP22, | | | | | | | |
| | | S100A8 | | | | | | | |
| 3- | BRD-K81647657 | CTSS, FABP4, | APOA1, | RXRA | | | | X | |
| Methyladenine | | IGF1, LPL, | APOC3, | | | | | | |
| | | S100A8, SLC6A8, | DLK1, | | | | | | |
| | | VCAN | RNASE2 | | | | | | |
| BRD- | BRD-K66908362 | CRABP2, ECM1, | ABCC3, | RXRA | | | | X | |
| K66908362 | | FSTL1, GPX1, | CYP3A5, | | | | | | |
| | | IGF1, IGFBP6, | WEE1 | | | | | | |
| | | LPL, MAOB, | | | | | | | |
| | | VCAN | | | | | | | |
| Hippeastrine | BRD-K71003802 | ALDH1A2, | HMOX1 | RXRA | | | | X | |
| Hydrobromide | | COLEC12, | | | | | | | |
| | | CRABP2, | | | | | | | |
| | | FABP4, GCLC, | | | | | | | |
| | | IGF1, S100A4, | | | | | | | |
| | | S100A8, | | | | | | | |
| | | SLC6A8, TPP1 | | | | | | | |
| Nicardipine | BRD-A26711594 | ALDH1A2, CTSB, | APOA1 | RXRA | | | | X | |
| Hydrochioride | | CTSS, FABP4, | | | | | | | |
| | | IGF1, MAOB, | | | | | | | |
| | | MMP12, S100A8, | | | | | | | |
| | | TPP1, VCAN | | | | | | | |
| 7488728 | BRD-K13794505 | ALDH1A2, | ABCC3, | RXRA | | | | X | |
| | | CCND1, | CXCL10, | | | | | | |
| | | COLEC12, CTSS, | DLK1, | | | | | | |
| | | LYZ | RNASE2, | | | | | | |
| | | | WEE1 | | | | | | |
| 7521700 | BRD-K96326565 | ALDH1A1, | | RXRA | | | | X | |
| | | CCNB1, FSTL1, | | | | | | | |
| | | HBEGF, NAV2, | | | | | | | |
| | | NPY, RXRA, | | | | | | | |
| | | S100A8, VCAN | | | | | | | |
| BJM-AF-64 | BRD-K14324645 | ECM1, FABP4, | CCL8, | RXRA | | | | X | |
| BRD- | | IGFBP6, LYZ, | DLK1 | | | | | | |
| K14324645 | | MMP12, PMP22, | | | | | | | |
| | | S100A8, VCAN | | | | | | | |
| BMS-754807 | BRD-K13049116 | ALDH1A1, | DLK1, | RXRA | | | | X | |
| | | COLEC12, CTSS, | IFNG | | | | | | |
| | | IGFBP6, MYCN, | | | | | | | |
| | | NPY, RXRA, | | | | | | | |
| | | S100A8 | | | | | | | |
| BRD- | BRD-A29426959 | ALDH1A1, | C1QA, | RXRA | | | | X | |
| A29426959 | | FLRT3, LPL, | CCL20, | | | | | | |
| | | PMP22, VCAN | CXCL10, | | | | | | |
| | | | DLK1, | | | | | | |
| | | | WEE1 | | | | | | |
| BRD- | BRD-K04430056 | CRABP2, FLRT3, | CYP3A5, | RXRA | | | | X | |
| K04430056 | | LPL, NDUFC2, | HMOX1 | | | | | | |
| | | NEDD9, S100A8, | | | | | | | |
| | | VCAN | | | | | | | |
| BRD- | BRD-K35638681 | ALDH1A2, | | RXRA | | | | X | |
| K35638681 | | CRABP2, CTSB, | | | | | | | |
| | | ECM1, FABP4, | | | | | | | |
| | | FSTL1, IGFBP6, | | | | | | | |
| | | PMP22, S100A8, | | | | | | | |
| | | TPP1 | | | | | | | |
| BRD- | BRD-K42471691 | FABP4, LCN2, | C1QA, | RXRA | | | | X | |
| K42471691 | | MAOB, MMP12, | DLK1 | | | | | | |
| | | PMP22, S100A8, | | | | | | | |
| | | VCAN | | | | | | | |
| BRD- | BRD-K56697208 | FABP4, HBEGF, | CCL8, | RXRA | | | | X | |
| K56697208 | | LCN2, LPL, LYZ, | CXCL10, | | | | | | |
| | | VCAN | HMOX1 | | | | | | |
| BRD- | BRD-K60729220 | ALDH1A1, | DLK1 | RXRA | | | | X | |
| K60729220 | | CTSB, FABP4, | | | | | | | |
| | | GCLC, MAOB, | | | | | | | |
| | | PMP22, RXRA, | | | | | | | |
| | | SLC6A8, | | | | | | | |
| | | SREBF1 | | | | | | | |
| BRD- | BRD-K94270326 | COLEC12, CTSS, | CYP3A5, | RXRA | | | | X | |
| K94270326 | | ECM1, FABP4, | DLK1 | | | | | | |
| | | LCN2, LPL, TPP1 | | | | | | | |
| BRD- | BRD-K98025142 | FABP1, FABP4, | ABCC3, | RXRA | | | | X | |
| K98025142 | | IL1A, LPL, RET, | DLK1 | | | | | | |
| | | S100A8, VCAN | | | | | | | |
| CC-100 | BRD-K35128472 | CCND1, CTSB, | CXCL10, | RXRA | | | | X | |
| | | GPX1, HOXA1, | RNASE2 | | | | | | |
| | | LPL, MAOB, | | | | | | | |
| | | MYCN, S100A8 | | | | | | | |
| GBR 12783 | BRD-K92015269 | ALDH1A1, | CCL8, | RXRA | | | | X | |
| | | CCND1, CRABP2, | CXCL10 | | | | | | |
| | | CTSB, ECM1, | | | | | | | |
| | | LYZ, VCAN | | | | | | | |
| Isradipine | BRD-A90799790 | ALDH1A2, | ABCC3, | RXRA | | | | X | |
| | | CRABP2, LCN2, | DLK1 | | | | | | |
| | | LPL, LYZ, | | | | | | | |
| | | MMP12, S100A8, | | | | | | | |
| | | VCAN | | | | | | | |
| Ivachtin | BRD-K64402243 | ALDH1A1, | C1QA | RXRA | | | | X | |
| | | CCNB1, ECM1, | | | | | | | |
| | | FABP6, IGF1, | | | | | | | |
| | | LCN2, RXRA, | | | | | | | |
| | | VCAN | | | | | | | |
| N6 - | BRD-A85234536 | CTSS, FABP4, | CCL20 | RXRA | | | | X | |
| Cyclopen- | | FLRT3, HBEGF, | | | | | | | |
| tyladenosine | | LPL, MAOB, | | | | | | | |
| | | NPY, VCAN | | | | | | | |
| Nisoxetine | BRD-A95696066 | ALDH1A1, | | RXRA | | | | X | |
| Hydrochioride | | ALDH1A2, | | | | | | | |
| | | ECM1, FABP4, | | | | | | | |
| | | GCLC, IGFBP6, | | | | | | | |
| | | LYZ, S100A8, | | | | | | | |
| | | SLC6A8 | | | | | | | |
| Nutlin-3 | BRD-A12230535 | ALDH1A1, | CCL20, | RXRA | | | | X | |
| | | COLEC12, | DLK1, | | | | | | |
| | | FABP1, FABP4, | RNASE2 | | | | | | |
| | | FLRT3, LPL, | | | | | | | |
| | | NAV2, VCAN | | | | | | | |
| OSSK_599080 | BRD-A83402799 | CLMN, IL1A, | RNASE2 | RXRA | | | | X | |
| | | LCN2, LPL, | | | | | | | |
| | | NEDD9, S100A8, | | | | | | | |
| | | SREBF1, VCAN | | | | | | | |
| RG 108 | BRD-K89391146 | ALDH1A1, | CYP35, | RXRA | | | | X | |
| | | ALDH1A2, ECM1, | TGFB2 | | | | | | |
| | | FABP6, LCN2, | | | | | | | |
| | | MAOB, S100A8, | | | | | | | |
| | | SLC6A8 | | | | | | | |
| RK-682 | BRD-A77349281 | ALDH1A2, | CCL20, | RXRA | | | | X | |
| | | CRABP2, ECM1, | DLK1, | | | | | | |
| | | FABP4, HBEGF, | HMOX1 | | | | | | |
| | | IGFBP6, VCAN | | | | | | | |
| SB 334867 | BRD-K41567364 | ALDH1A2, | ABCC3 | RXRA | | | | X | |
| | | COLEC12, | HMOX1 | | | | | | |
| | | FABP4, FABP6, | | | | | | | |
| | | GAS6, S100A8, | | | | | | | |
| | | SLC6A8, VCAN | | | | | | | |
| AZD8055 | BRD-K69932463 | CCP110, CD24, | AQP3, | TCF7L2 | | | | | X |
| | | EGFR, ENPP4, | CDKN1A, | | | | | | |
| | | ERBB3, EVI2A, | ID1, | | | | | | |
| | | GSN, MAN1A1, | ID3, | | | | | | |
| | | MBP, NPC1, | ID4 | | | | | | |
| | | PPP1R16B, | | | | | | | |
| | | RAP2A, RNF13, | | | | | | | |
| | | SPP1 | | | | | | | |
| BRD- | BRD-K90999434 | ACSL3, APOD, | AQP3, | TCF7L2 | | | | | X |
| K90999434 | | CCND2, CD24, | ID1, | | | | | | |
| | | CCP110, CDK19, | ID3, ID4 | | | | | | |
| | | CPD, CSRP1, | | | | | | | |
| | | CYP51A1, DDC, | | | | | | | |
| | | FGFR2, GSN, | | | | | | | |
| | | HSPA2, IDH1, | | | | | | | |
| | | IRS2, LAMP1, | | | | | | | |
| | | MAP7, MBP, ME1, | | | | | | | |
| | | MYO6, NPC2, | | | | | | | |
| | | POLR1D, | | | | | | | |
| | | SECISBP2L, | | | | | | | |
| | | SEMA4D, | | | | | | | |
| | | SLC12A2, SPP1, | | | | | | | |
| | | ST18 | | | | | | | |
| NSC 23766 | BRD-A80213327 | ACAA1, BIN1, | AQP3, | TCF7L2 | | | | | X |
| | | CCND2, CD24, | BMP4 | | | | | | |
| | | CDK19, FTH1, | | | | | | | |
| | | GSN, HSPA2, | | | | | | | |
| | | MAP4K4, MAP7, | | | | | | | |
| | | ME1, MYO6, | | | | | | | |
| | | PPP1R16B, | | | | | | | |
| | | RAP2A, ST18 | | | | | | | |
| Teniposide | BRD-A35588707 | ACSL1, ALOX5, | ID1, ID4, | TCF7L2 | | | | | X |
| | | CCND2, CCP110, | PCK1 | | | | | | |
| | | DHRS7, GSN, | | | | | | | |
| | | JAM3, MAP4K4, | | | | | | | |
| | | MAP7, MCAM, | | | | | | | |
| | | PAPSS1, PELI1, | | | | | | | |
| | | RNF13, | | | | | | | |
| | | SECISBP2L | | | | | | | |
| BAS | BRD-K17119186 | ACSL3, ALOX5, | ID2, ID3, | TCF7L2 | | | | | X |
| 00535043 | | APOD, | PCK1 | | | | | | |
| | | CAMK2N1, | | | | | | | |
| | | CYP51A1, | | | | | | | |
| | | DHCR24, FTH1, | | | | | | | |
| | | GLUL, IDH1, | | | | | | | |
| | | IRS2, MMP7, | | | | | | | |
| | | NPC2, SPP1, | | | | | | | |
| | | UBE2G1 | | | | | | | |
| BRD- | BRD-K50177987 | ANXA1, APOD, | AQP3, | TCF7L2 | | | | | X |
| K50177987 | | CCP110, EGFR, | ID1, | | | | | | |
| | | ENPP2, FGFR2, | ID2, ID3 | | | | | | |
| | | GLUL, JAM3, | | | | | | | |
| | | KAT2B, PLP1, | | | | | | | |
| | | QKI, SH3GL3, | | | | | | | |
| | | TCF7L2, TNS1 | | | | | | | |
| BRD- | BRD-K76568384 | ACSL1, ADO, | ID3, | TCF7L2 | | | | | X |
| K76568384 | | CD24, CPD, | PCK1 | | | | | | |
| | | DHCR24, EGFR, | | | | | | | |
| | | ELOVL1, GLUL, | | | | | | | |
| | | KIF5B, LAMP1, | | | | | | | |
| | | MAP4K4, MAP7, | | | | | | | |
| | | MMP7, SLC12A2, | | | | | | | |
| | | SLC27A2, STK39, | | | | | | | |
| | | TMEM123 | | | | | | | |
| 2541665-P2 | BRD-K61053657 | ALOX5, APOD, | AQP3, | TCF7L2 | | | | | X |
| | | AQP9, ASPH, | CDKN1B, | | | | | | |
| | | DEGS1, ENPP2, | ID1, | | | | | | |
| | | ENPP4, ERBB3, | ID4 | | | | | | |
| | | FA2H, FGFR2, | | | | | | | |
| | | GLUL, HIPK2, | | | | | | | |
| | | HSPA2, IPO13, | | | | | | | |
| | | KIF5B, KTN1, | | | | | | | |
| | | MAL, MAP4K4, | | | | | | | |
| | | ME1, MYO6, | | | | | | | |
| | | PLEKHB1, PLP1, | | | | | | | |
| | | PPP1R16B, QKI, | | | | | | | |
| | | RAP2A, RNASE4, | | | | | | | |
| | | SPP1, TCF7L2 | | | | | | | |
| BRD- | BRD-K34495954 | APOD, CD24, | | TCF7L2 | | | | | X |
| K34495954 | | CSRP1, ENPP4, | | | | | | | |
| | | FGFR2, GSN, | | | | | | | |
| | | HMGCS2, HSPA2, | | | | | | | |
| | | JAG1, LAMP1, | | | | | | | |
| | | MAN1A1, MAP7, | | | | | | | |
| | | MMP7, NFASC, | | | | | | | |
| | | PAPSS1, SLC12A2 | | | | | | | |
| BRD- | BRD-K59488055 | ACSL3, APOD, | BMP4 | TCF7L2 | | | | | X |
| K59488055 | | CDK19, CSRP1, | | | | | | | |
| | | CYP51A1, | | | | | | | |
| | | DHCR24, FTH1, | | | | | | | |
| | | IDH1, IRS1, IRS2, | | | | | | | |
| | | MBP, NUDT4, | | | | | | | |
| | | PLP1, QKI, | | | | | | | |
| | | SLC12A2 | | | | | | | |
| DM161 | BRD-K95212245 | BIN1, CCP110, | ID1, ID2, | TCF7L2 | | | | | X |
| BRD- | | CD24, DOCK9, | ID4 | | | | | | |
| K95212245 | | EVI2A, FA2H, | | | | | | | |
| | | HIPK2, KIF5B, | | | | | | | |
| | | MAG, MAP4K4, | | | | | | | |
| | | MAP7, MBP, | | | | | | | |
| | | MOBP, MYO6, | | | | | | | |
| | | NPC1, STK39 | | | | | | | |
| Idazoxan | BRD-A18696154 | ANXA1, ASPH, | KDM4B | TCF7L2 | | | | | X |
| Hydrochloride | | ENPP4, EPAS1, | | | | | | | |
| | | FGFR2, HSPA2, | | | | | | | |
| | | MAN1A1, MAP7, | | | | | | | |
| | | MOBP, MOG, | | | | | | | |
| | | PLP1, PPP1R16B, | | | | | | | |
| | | QKI, RAB33A, | | | | | | | |
| | | SH3GL3, SOX10, | | | | | | | |
| | | ST18 | | | | | | | |
| NCGC00182823- | BRD-K74348865 | ACSL1, APOD, | BMP4, | TCF7L2 | | | | | X |
| 01 | | BIN1, CD24, | PCK1 | | | | | | |
| | | CSRP1, EGFR, | | | | | | | |
| | | ENPP2, IRS2, | | | | | | | |
| | | LAMP1, MAP7, | | | | | | | |
| | | MBP, MCAM, | | | | | | | |
| | | PLP1, SECISBP2L, | | | | | | | |
| | | SLC27A2, TWF1 | | | | | | | |
| Thiazolopyrimidine | BRD-A75517195 | CCND2, CPD, | AQP3, | TCF7L2 | | | | | X |
| | | DOCK9, EGFR, | ID1 | | | | | | |
| | | ENPP2, EPAS1, | | | | | | | |
| | | FA2H, FGFR2, | | | | | | | |
| | | GCLM, HSPA2, | | | | | | | |
| | | JAG1, MAN1A1, | | | | | | | |
| | | ME1, MMP7, | | | | | | | |
| | | RAP2A, | | | | | | | |
| | | TMEM123 | | | | | | | |
| Wortmannin | BRD-A75409952 | APOD, CD24, | ID1 | TCF7L2 | | | | | X |
| | | DHRS7, EPAS1, | | | | | | | |
| | | IPO13, MAN1A1, | | | | | | | |
| | | MAP7, MOBP, | | | | | | | |
| | | MOG, MYO6, | | | | | | | |
| | | NPC1, PELI1, QKI, | | | | | | | |
| | | RNF13, SMAD7, | | | | | | | |
| | | WISP2 | | | | | | | |
| 1503640 | BRD-A66395008 | APOD, ASPH, | AQP3 | TCF7L2 | | | | | X |
| | | CCND2, CD24, | | | | | | | |
| | | DHCR24, EGFR, | | | | | | | |
| | | FGFR2, GLUL, | | | | | | | |
| | | GSN, HSPA2, | | | | | | | |
| | | LAMP1, MBP, | | | | | | | |
| | | TNS1 | | | | | | | |
| BRD- | BRD-A19195498 | ACSL1, ASPA, | ID3 | TCF7L2 | | | | | X |
| A19195498 | | CD24, DDC, | | | | | | | |
| | | DEGS1, DHCR24, | | | | | | | |
| | | FTH1, GLUL, | | | | | | | |
| | | MAP7, NPC1, | | | | | | | |
| | | NPC2, PRRG1, | | | | | | | |
| | | RAP2A, | | | | | | | |
| | | SLC27A2, | | | | | | | |
| | | SRD5A1 | | | | | | | |
| BRD- | BRD-A94413429 | ACSL3, CD24, | ID1, ID2 | TCF7L2 | | | | | X |
| A94413429 | | CYP51A1, | | | | | | | |
| | | DHCR24, IDH1, | | | | | | | |
| | | IRS2, JAG1, | | | | | | | |
| | | MAN1A1, ME1, | | | | | | | |
| | | MMP7, MYO6, | | | | | | | |
| | | SLC12A2 | | | | | | | |
| BRD- | BRD-K21565985 | ACSL1, ADO, | ID4, | TCF7L2 | | | | | X |
| K21565985 | | APOD, CCND2, | PCK1 | | | | | | |
| | | DHRS7, EGFR, | | | | | | | |
| | | ENPP2, FTH1, | | | | | | | |
| | | GLUL, IRS2, | | | | | | | |
| | | LDLRAP1, MAP7, | | | | | | | |
| | | MMP7, SLC12A2, | | | | | | | |
| | | STK39 | | | | | | | |
| BRD- | BRD-K55612480 | ANXA1, | FBP1, | TCF7L2 | | | | | X |
| K55612480 | | CAMK2N1, | IDI, ID2 | | | | | | |
| | | CCND2, CD24, | | | | | | | |
| | | CPD, EPAS1, | | | | | | | |
| | | EVI2A, FTH1, | | | | | | | |
| | | GLUL, IPO13, | | | | | | | |
| | | MAN1A1, MAP7, | | | | | | | |
| | | RALGDS, TWF1 | | | | | | | |
| BRD- | BRD-K61217870 | ACSL1, APOD, | ID1, ID3 | TCF7L2 | | | | | X |
| K61217870 | | BIN1, CARHSP1, | | | | | | | |
| | | CSRP1, DDC, | | | | | | | |
| | | EGFR, ENPP2, | | | | | | | |
| | | KIF5B, KTN1, | | | | | | | |
| | | LAMP1, SLC27A2, | | | | | | | |
| | | TBC1D5 | | | | | | | |
| BRD- | BRD-K63326650 | ANXA1, CAP1, | AQP3 | TCF7L2 | | | | | X |
| K63326650 | | CD24, DDC, | | | | | | | |
| | | EPAS1, FGFR2, | | | | | | | |
| | | GSN, IPO13, IRS1, | | | | | | | |
| | | JAG1, MAL, | | | | | | | |
| | | MAN1A1, MAP7, | | | | | | | |
| | | PPP1R16B, | | | | | | | |
| | | RAB31 | | | | | | | |
| BRD- | BRD-K71670746 | ACSL3, APOD, | CDKN1B, | TCF7L2 | | | | | X |
| K71670746 | | ASPH, CPD, | ID1, | | | | | | |
| | | CYP51A1, IRS1, | ID3 | | | | | | |
| | | JAG1, MAP7, | | | | | | | |
| | | MBP, ME1, | | | | | | | |
| | | RAB31, SLC27A2 | | | | | | | |
| BRD- | BRD-K76587808 | APOD, ARAP2, | ID1, | TCF7L2 | | | | | X |
| K76587808 | | ARHGEF10, CD24, | ID2, | | | | | | |
| | | CTNNAL1, GSN, | ID3, | | | | | | |
| | | MAP4K4, MAP7, | ID4 | | | | | | |
| | | MYO6, PLAT, | | | | | | | |
| | | PTPN11, | | | | | | | |
| | | SECISBP2L | | | | | | | |
| BRD- | BRD-K76896292 | ASPH, CAMK2N1, | AQP3, | TCF7L2 | | | | | X |
| K76896292 | | CD24, CPD, GJB1, | BMP4 | | | | | | |
| | | HMGCS2, JAG1, | | | | | | | |
| | | MAN1A1, MAP7, | | | | | | | |
| | | MBP, MCAM, | | | | | | | |
| | | PELI1, QKI, | | | | | | | |
| | | RCBTB1, | | | | | | | |
| | | SLC12A2 | | | | | | | |
| BRD- | BRD-K93480852 | ACSL1, ADO, | CDKN1B, | TCF7L2 | | | | | X |
| K93480852 | | APOD, CCND2, | ID1, | | | | | | |
| | | CSRP1, CYP51A1, | ID4 | | | | | | |
| | | DHCR24, FTH1, | | | | | | | |
| | | IPO13, MAP7, | | | | | | | |
| | | RCBTB1, ST18 | | | | | | | |
| BRD- | BRD-K98991361 | APOD, CCP110, | ID1 | TCF7L2 | | | | | X |
| K98991361 | | ERBB3, FTH1, | | | | | | | |
| | | GLUL, MAP7, | | | | | | | |
| | | MBP, ME1, | | | | | | | |
| | | PICALM, | | | | | | | |
| | | PLEKHB1, PLP1, | | | | | | | |
| | | POLR1D, | | | | | | | |
| | | SLC27A2, SOX10 | | | | | | | |
| INK-128 | BRD-K59317601 | APOD, | ID1, ID2, | TCF7L2 | | | | | X |
| | | CAMK2N1, | ID3 | | | | | | |
| | | CD24, DHRS7, | | | | | | | |
| | | ERBB3, GSN, | | | | | | | |
| | | HSPA2, IRS1, | | | | | | | |
| | | IRS2, MMP7, | | | | | | | |
| | | MYC, MYO6, | | | | | | | |
| | | POLR1D | | | | | | | |
| MLS- | BRD-K35035132 | ALOX5, APOD, | ID4 | TCF7L2 | | | | | X |
| 0327420.0002 | | ASPA, CCP110, | | | | | | | |
| | | CD24, ENPP2, | | | | | | | |
| | | GSN, HSPA2, | | | | | | | |
| | | IPO13, JAG1, | | | | | | | |
| | | MAN1A1, MBP, | | | | | | | |
| | | MMP7, SLC12A2, | | | | | | | |
| | | SMAD7, STK39 | | | | | | | |
| MW-Ras9 | BRD-K50505048 | ACSL1, APOD, | BMP4 | TCF7L2 | | | | | X |
| | | CAMK2N1, | | | | | | | |
| | | CCP110, CSRP1, | | | | | | | |
| | | GSN, KIF5B, | | | | | | | |
| | | MAP4K4, MBP, | | | | | | | |
| | | PLP1, PTPN11, | | | | | | | |
| | | SLC12A2, SOX10, | | | | | | | |
| | | STK39, SYPL1 | | | | | | | |
| NCGC00182845- | BRD-A83993876 | ARAP2, BIN1, | CDKN1B, | TCF7L2 | | | | | X |
| 01 | | CAMK2N1, CPD, | ID3 | | | | | | |
| | | CSRP1, ENPP2, | | | | | | | |
| | | ERBB3, FGFR2, | | | | | | | |
| | | GSN, MAP4K4, | | | | | | | |
| | | MAP7, SEMA4D, | | | | | | | |
| | | SLC27A2 | | | | | | | |
| Sertraline | BRD-K82036761 | ACSL3, BIN1, | AQP3 | TCF7L2 | | | | | X |
| | | CCND2, CD24, | | | | | | | |
| | | CYP51A1, | | | | | | | |
| | | DHCR24, FA2H, | | | | | | | |
| | | FTH1, IDH1, IRS1, | | | | | | | |
| | | IRS2, MBP, ME1, | | | | | | | |
| | | NPC1, SPP1, | | | | | | | |
| | | SRD5A1 | | | | | | | |
| alproic Acid | BRD-K41260949 | APOD, ASPH, | AQP3, | TCF7L2 | | | | | X |
| | | CAMK2N1, | CDKN1B | | | | | | |
| | | CCP110, DHRS7, | | | | | | | |
| | | EGFR, ENPP2, | | | | | | | |
| | | HSPA2, JAG1, | | | | | | | |
| | | MBP, QKI, | | | | | | | |
| | | SECISBP2L | | | | | | | |
| BRD- | BRD-K04853698 | ACSL3, ARAP2, | ID2 | TCF7L2 | | | | | X |
| K04853698 | | CD24, CREB3L2, | | | | | | | |
| | | DHCR24, EPAS1, | | | | | | | |
| | | FTH1, GCLM, | | | | | | | |
| | | IRS2, JAG1, ME1, | | | | | | | |
| | | MYC, NPC1 | | | | | | | |
| BRD- | BRD-K74761218 | CAMK2N1, | ID4 | TCF7L2 | | | | | X |
| K74761218 | | CCND2, CD24, | | | | | | | |
| | | CDK19, CHMP1B, | | | | | | | |
| | | CPD, EPAS1, | | | | | | | |
| | | ERBB3, GSN, | | | | | | | |
| | | JAG1, MAN1A1, | | | | | | | |
| | | MAP7, NPC1, | | | | | | | |
| | | PAPSS1, PELI1, | | | | | | | |
| | | PLEKHB1 | | | | | | | |
| Dasatinib | BRD-K49328571 | ACSL3, | | TCF7L2 | | | | | X |
| | | CAMK2N1, CD24, | | | | | | | |
| | | CPD, CYP51A1, | | | | | | | |
| | | DHCR24, ENPP2, | | | | | | | |
| | | EPAS1, FTH1, | | | | | | | |
| | | GLUL, IRS1, IRS2, | | | | | | | |
| | | MMP7, SPP1 | | | | | | | |
| Geldanamycin | BRD-A19500257 | APOD, CAMK2N1, | ID1, ID3 | TCF7L2 | | | | | X |
| | | CPD, FTH1, GLUL, | | | | | | | |
| | | HSPA2, MAN1A1, | | | | | | | |
| | | ME1, MYO6, | | | | | | | |
| | | PPP1R16B, | | | | | | | |
| | | SECISBP2L, | | | | | | | |
| | | SLC12A2, | | | | | | | |
| | | SNAP23, SORBS3 | | | | | | | |
| JW-7-24-1 | BRD-K57282030 | APOD, CYP51A1, | FH, ID1, | TCF7L2 | | | | | X |
| | | DHCR24, IRS1, | PCK1 | | | | | | |
| | | IRS2, NKX2-1, | | | | | | | |
| | | NPC1, NPC2, | | | | | | | |
| | | POLR1D, RAB31 | | | | | | | |

In one embodiment, the one or more modulators stimulate Notch signaling.

Modulators that stimulate Notch signaling may upregulate, without limitation, one or more genes selected from the group consisting of ACAA1, ADCY9, ALOX5, CD24, CPD, CYP51A1, DHCR24, EPAS1, ERBB3, GSN, GNAI1, HES1, IDH1, JAG1, MAN1A1, ME1, NOTCH1, NOTCH3, NPC1, PAPSS1, PLAT, RAB31, SRD5A1, CAMK2N1, CAP1, CCND2, DOCK9, FGFR2, MAP7, PELI1, PPP1R16B, PRKAR2B, RAP1GAP, RPS6KA1, ADRB2, APLNR, CALM1, JAG2, NOTCH2, CAMK2G, TBXA2R, ALDH1A2, ECM1, FSTL1, HBEGF, HES1IGFBP6, LCN2, LPL, S100A8, APH1A, COL15A1, DCN, DSC2, GADD45A, GPR37, SAT1, and IER3 and/or downregulate one or more genes selected from the group consisting of DUSP1, DUSP4, ID4, KLF2, CCL8, MARCO, GPNMB, and RGS4.

Exemplary modulators of Notch signaling include, without limitation, one or more modulators selected from the group consisting of Trichostatin A, Vorinostat, BJM-ctd2-9, Pifithrin-a, 5587525, Acefylline, BL-095, BMS 191011, BRD-A21723284, BRD-K02275692, BRD-K11540476, BRD-K11778076, BRD-K15563106, BRD-K26573499, BRD-K28075147, BRD-K37618799, BRD-K38519699, BRD-K54708045, BRD-K70947604, BRD-K86108784, BRD-K93875449, BRD-K96041033, IKK Inhibitor X, L-750,667, L-sulforophane, Metolazone, MLS-0014097.0001, Naloxone Hydrochloride, NRB 04155, Prostaglandin A1, RG-13022, RS 16566 Hydrochloride, STOCK2S-25759, T5345967, Triacsin-c, Tyrphostin B44 (+) Enantiomer, YM-155, BRD-K00313977, BRD-K43620258, BX-795, Cefixime, Cercosporin, Methylene Blue, Selamectin, and VX-680.

In another embodiment, the one or more modulators stimulate cAMP mediated signaling.

Modulators that stimulate cAMP signaling may upregulate, without limitation, one or more genes selected from the group consisting of ACAA1, ADCY9, ALOX5, CD24, CPD, CYP51A1, DHCR24, EPAS1, ERBB3, GSN, GNAI1, HES1, IDH1, JAG1, MAN1A1, ME1, NOTCH1, NOTCH3, NPC1, PAPSS1, PLAT, RAB31, SRD5A1, CAMK2N1, CAP1, CCND2, DOCK9, FGFR2, MAP7, PELI1, PPP1R16B, PRKAR2B, RAP1GAP, RPS6KA1, AGTR1, CFB, COL15A1, DCN, FAP, LXN, PTGER2, SAT1, SERPINE2, ADRB2, C4BPA, CALML5, CRLF1, CRYAB, GPR183, HCAR3, LPAR1, LUM, P2RY14, PDLIM7, SRC, APLNR, CALM1, JAG2, NOTCH2, CAMK2G, TBXA2R, ACSL1, APOD, ASPA, CAMK2N1, CCP110, ENPP2, FTH1, GNAS, HSPA2, IPO13, IRS2, MOBP, PLP1, PRKAR1A, PTGER4, ADORA2B, CHRM3, CNR1, VIPR1, P2RY13, PTGDR, ADCY7, GABBR2, PTGER3, PRKACB, NPR3, CREB1, ADCY1, and STAT3 and/or downregulate one or more genes selected from the group consisting of DUSP1, DUSP4, ID4, CCND1, DUSP6, PKIA, PKIG, PDE2A, RGS2, RGS4, ID1, ID2, PPP3CA, CREM, PKIA, PKIG, and PPP3CA.

Exemplary modulators of cAMP signaling include, without limitation, one or more modulators selected from the group consisting of Trichostatin A, BRD-K08438429, Ichthynone, Vorinostat, BRD-K30523950, BRD-K64245000, NF 449, BRD-A34751532, BRD-K63938928, BRD-K64402243, 7b-cis, BRD-A36318220, BRD-K09549677, BRD-K71430621, BRD-K74212935, BRD-K93623754, Bumetanide, Chloroquine Diphosphate, Laudanosine (R,S), PD-184352, PRL-3 Inhibitor I, and Troxipide.

In yet another embodiment, the one or more modulators inhibit CIP2A signaling.

Modulators that inhibit CIP2A signaling may upregulate, without limitation, one or more genes selected from the group consisting of AGTR1, CFB, COL15A1, DCN, FAP, GNAI1, LXN, PRKAR2B, PTGER2, SAT1, SERPINE2, ADRB2, C4BPA, CALML5, CRLF1, CRYAB, GNAI1, GPR183, HCAR3, LPAR1, LUM, P2RY14, PDLIM7, SRC, DSC2, GADD45A, GPR37, HES1, IER3, JAG1, NOTCH1, ECM1, FABP1, GAS6, GPX1, HIST1H2BK, PLAUR, S100A8, SLC22A18, VCAN, ALDH1A2, CCND1, CRABP2, FLRT3, IGFBP6, LPL, LYZ, RET, SNCA, SLC22A4, NPTX1, FAP, LRP4, KIAA1324, SLC12A8, TUBA4A, RHOC, PDGFRB, EBI3, and ENO3 and/or downregulate one or more genes selected from the group consisting of CCND1, DUSP6, PKIA, PKIG, PDE2A, RGS2, RGS4, GPNMB, C1QA, CCL8, DLK1, E2F2, CXCL10, MFAP5, ACTG2, ZDHHC11, MYC, SLC25A4, PDE2A, ZDHHC11, RAB31, GRSF1, MYC, and PDK4.

Exemplary modulators of CIP2A signaling include, without limitation, one or more modulators selected from the group consisting of BRD-K08438429, Ichthynone, BJM-ctd2-9BRD-K51126483, DO 897/99, BRD-K44276885, Arachidonyl trifluoro-methyl ketone, BRD-A25234499, BRD-A69636825, BRD-K34170797, BRD-K46445327, BRD-K49807497, BRD-K68548958, BRD-K71879957, Calcipotriol, GANT 58, Lamivudine, Radicicol, 71748, 10006734, BRD-A36630025, BRD-A41250203, BRD-K00317371, BRD-K13810148, BRD-K41429297, BRD-K49010888, BRD-K50214219, BRD-K99135512, COT-10b, Estriol Methyl Ether, GDC-0941, HDAC6 inhibitor ISOX, KUC104141, KUC104141N, Meclofenamate Sodium, MLS-0315803, NCGC00182837-01, NCGC00229626-01, NCGC00241438-01, Pipamperone, PJ 34 Hydrochloride, PX12, SU6668, and XMD11-85H.

In a further embodiment, the one or more modulators stimulate RXRA signaling.

Modulators that stimulate RXRA signaling may upregulate, without limitation, one or more genes selected from the group consisting of CFB, CRYAB, DSC2, ECM1, FABP1, GAS6, GPX1, HIST1H2BK, PLAUR, S100A8, SLC22A18, ALDH1A2, C4BPA, CCND1, CRABP2, FLRT3, IGFBP6, LPL, LYZ, PLAUR, RET, SERPINE2, VCAN, FSTL1, HBEGF, HES1IGFBP6, JAG1, LCN2, NOTCH2, NOTCH3, ALDH1A1, CD24, COLEC12, DDC, EGFR, ENPP2, EPAS1, FA2H, FABP4, GCLC, MAG7, MBP, MCAM, NPY, PDGFA, PMP22, QKI, SLC6A8, WISP2, CDK19, CREB3L2, DOCK9, ENPP4, IGF1, KAT2B, MAN1A1, NFASC, NKX2-1, OLIG2, PLP1, SH3GL3, SLC12A2, SLC27A2, SOX10, CTSB, MMP12, CCNB1, IL1B, MAOB, MMP2, CTSS, S100A4, TPP1, NAV2, RXRA, MYCN, NDUFC2, NEDD9, ILIA, RET, HOXA1, SLC6A8, CLMN, FABP6, and SREBF1 and/or downregulate one or more genes selected from the group consisting of ABCC3, CYP3A5, ABCC3, HMOX1, APOA1, APOC3, DLK1, RNASE2, WEE1, CXCL10, CCL8, IFNG, C1QA, CCL20, CYP3A5, TGFB2, E2F2, MFAP5, MARCO, AQP3, BMP4, ID2, and ID3.

Exemplary modulators of RXRA signaling include, without limitation, one or more modulators selected from the group consisting of BRD-K51126483, DO 897/99, Pifithrin-a, CI 976, Rolipram, BRD-K90610876, L5288-1MG, 1,25 DIHYDROXYVITAMIN D3, 3-Deoxydenosine, 3-Methyladenine, BRD-K66908362, Hippeastrine Hydrobromide, Nicardipine Hydrochloride, 7488728, 7521700, BJM-AF-64, BRD-K14324645, BMS-754807, BRD-A29426959, BRD-K04430056, BRD-K35638681, BRD-K42471691, BRD-K56697208, BRD-K60729220, BRD-K94270326, BRD-K98025142, CC-100, GBR 12783, Isradipine, Ivachtin, N6-Cyclopentyladenosine, Nisoxetine Hydrochloride, Nutlin-3, OSSK 599080, RG 108, RK-682, and SB 334867.

In another embodiment, the one or more modulators stimulate TCF7L2 signaling.

Modulators that stimulate TCF7L2 signaling may upregulate, without limitation, one or more genes selected from the group consisting of ACAA1, ADCY9, ALOX5, CD24, CPD, CYP51A1, DHCR24, EPAS1, ERBB3, GSN, GNAI1, HES1, IDH1, JAG1, MAN1A1, MEL NOTCH1, NOTCH3, NPC1, PAPSS1, PLAT, SRD5A1, CAMK2N1, CAP1, CCND2, DOCK9, FGFR2, MAP7, PELI1, PPP1R16B, PRKAR2B, RAP1GAP, RPS6KA1, ACSL1, ADORA2B, ADRB2, APOD, ASPA, CCP110, ENPP2, FTH1, GNAS, HSPA2, IPO13, IRS2, MOBP, PLP1, PRKAR1A, PTGER4, ALDH1A1, CCND1, COLEC12, CRABP2, DDC, EGFR, FA2H, FABP4, GCLC, GPX1, HBEGF, LCN2, LPL, MAG7, MBP, MCAM, NPY, PDGFA, PMP22, QKI, S100A8, SLC6A8, WISP2, ALDH1A2, CDK19, CREB3L2, ENPP4, IGF1, KAT2B, NKX2-1, OLIG2, SH3GL3, SLC12A2, SLC27A2, SOX10, BIN1, CDK19, MAP4K4, MYO6, RAP2A, ST18, CCP110, DHRS7, JAM3, MCAM, PAPSS1, RNF13, SECISBP2L, ACSL3, GLUL, MMP7, NPC2, SPP1, UBE2G1, ANXA1, TCF7L2, TNS1, ADO, ELOVL1, KIF5B, LAMP1, STK39, TMEM123, AQP9, ASPH, DEGS1, HIPK2, KTN1, MAL, PLEKHB1, RNASE4, CSRP1, HMGCS2, NFASC, IRS1, NUDT4, EVI2A, MAG, MOG, RAB33A, TWF1, GCLM, SMAD7, PRRG1, LDLRAP1, EVI2A, RALGDS, CARHSP1, TBC1D5, ARAP2, ARHGEF10, CTNNAL1, PTPN11, GJB1, HMGCS2, RCBTB1, PICALM, POLR1D, MYC, ALOX5, SYPL1, SEMA4D, CHMP1B, SNAP23, SORBS3, and RAB31 and/or downregulate one or more genes selected from the group consisting of ID1, ID4, PCK1, ID3, AQP3, CDKN1B, BMP4, KDM4B, FBP1, DUSP1, DUSP4, PKIG, PPP3CA, ABCC3, CCL20, TGFB2, DLK1, WEE1, APOA1, CXCL10, DLK1, ID2, and FH.

Exemplary modulators include, without limitation, one or more modulators selected from the group consisting of Trichostatin A, BRD-K30523950, CI 976, Rolipram, AZD8055, BRD-K90999434, NSC 23766, Teniposide, BAS 00535043, BRD-K50177987, BRD-K76568384, 2541665-P2, BRD-K34495954, BRD-K59488055, DM161, BRD-K95212245, Idazoxan Hydrochloride, NCGC00182823-01, Thiazolopyrimidine, Wortmannin, 1503640, BRD-A19195498, BRD-A94413429, BRD-K21565985, BRD-K55612480, BRD-K61217870, BRD-K63326650, BRD-K71670746, BRD-K76587808, BRD-K76896292, BRD-K93480852, BRD-K98991361, INK-128, MLS-0327420.0002, MW-Ras9, NCGC00182845-01, Sertraline, Valproic Acid, BRD-K04853698, BRD-K74761218, Dasatinib, Geldanamycin, and JW-7-24-1.

Conditions mediated by a loss of myelin or a loss of oligodendrocytes that can be treated in accordance with the methods of the present application include hypomyelination disorders and demyelinating disorders. In one embodiment of the present application, the condition is an inflammatory demyelination condition, such as e.g., multiple sclerosis, neuromyelitis optica, transverse myelitis, and optic neuritis. In another embodiment of the present application, the myelin-related disorder is a vascular leukoencephalopathy, such as e.g., subcortical stroke, diabetic leukoencephalopathy, hypertensive leukoencephalopathy, age-related white matter disease, and spinal cord injury. In another embodiment of the present application, the myelin-related condition is a radiation- or chemotherapy-induced demyelination condition. In another embodiment the conditions is post-infectious or post-vaccinial leukoencephalitis. In another embodiment of the present application, the myelin-related disorder is a pediatric leukodystrophy, such as e.g., Pelizaeus-Merzbacher Disease, Tay-Sach Disease, Sandhoff's gangliosidoses, Krabbe's disease, metachromatic leukodystrophy, mucopolysaccharidoses, Niemann-Pick A disease, adrenoleukodystrophy, Canavan's disease, Vanishing White Matter Disease, and Alexander Disease. In yet another embodiment of the present application, the myelin-related condition is periventricular leukomalacia or cerebral palsy. In another embodiment, the condition is a lysosomal storage disease, congenital demyelination, or vascular demyelination.

In one embodiment, the method described supra further includes administering to the selected subject a preparation of human glial progenitor cells.

The human glial progenitor cells may be derived from any suitable source of glial cells, such as, for example and without limitation, human induced pluripotent stem cells (iPSCs), embryonic stem cells, fetal tissue, and/or astrocytes as described in more detail below.

iPSCs are pluripotent cells that are derived from non-pluripotent cells, such as somatic cells. For example, and without limitation, iPSCs can be derived from tissue, peripheral blood, umbilical cord blood, and bone marrow (see e.g., Cai et al., “Generation of Human Induced Pluripotent Stem Cells from Umbilical Cord Matrix and Amniotic Membrane Mesenchymal Cells,” J. Biol. Chem. 285(15):112227-11234 (2110); Giorgetti et al., “Generation of Induced Pluripotent Stem Cells from Human Cord Blood Cells with only Two Factors: Oct4 and Sox2,” Nat. Protocol. 5(4):811-820 (2010); Streckfuss-Bomeke et al., “Comparative Study of Human-Induced Pluripotent Stem Cells Derived from Bone Marrow Cells, Hair Keratinocytes, and Skin Fibroblasts,” Eur. Heart J. doi:10.1093/eurheartj/ehs203 (Jul. 12, 2012); Hu et al., “Efficient Generation of Transgene-Free Induced Pluripotent Stem Cells from Normal and Neoplastic Bone Marrow and Cord Blood Mononuclear Cells,” Blood doi:10.1182/blood-2010-07-298331 (Feb. 4, 2011); Sommer et al., “Generation of Human Induced Pluripotent Stem Cells from Peripheral Blood using the STEMCCA Lentiviral Vector,” J. Vis. Exp. 68:e4327 doi:10.3791/4327 (2012), which are hereby incorporated by reference in their entirety). The somatic cells are reprogrammed to an embryonic stem cell-like state using genetic manipulation. Exemplary somatic cells suitable for the formation of iPSCs include fibroblasts (see e.g., Streckfuss-Bomeke et al., “Comparative Study of Human-Induced Pluripotent Stem Cells Derived from Bone Marrow Cells, Hair Keratinocytes, and Skin Fibroblasts,” Eur. Heart J. doi:10.1093/eurheartj/ehs203 (2012), which is hereby incorporated by reference in its entirety), such as dermal fibroblasts obtained by a skin sample or biopsy, synoviocytes from synovial tissue, keratinocytes, mature B cells, mature T cells, pancreatic 0 cells, melanocytes, hepatocytes, foreskin cells, cheek cells, or lung fibroblasts.

Methods of producing induced pluripotent stem cells are known in the art and typically involve expressing a combination of reprogramming factors in a somatic cell. Suitable reprogramming factors that promote and induce iPSC generation include one or more of Oct4, Klf4, Sox2, c-Myc, Nanog, C/EBPα, Esrrb, Lin28, and Nr5a2. In certain embodiments, at least two reprogramming factors are expressed in a somatic cell to successfully reprogram the somatic cell. In other embodiments, at least three reprogramming factors are expressed in a somatic cell to successfully reprogram the somatic cell.

iPSCs may be derived by methods known in the art, including the use integrating viral vectors (e.g., lentiviral vectors, inducible lentiviral vectors, and retroviral vectors), excisable vectors (e.g., transposon and floxed lentiviral vectors), and non-integrating vectors (e.g., adenoviral and plasmid vectors) to deliver the genes that promote cell reprogramming (see e.g., Takahashi and Yamanaka, Cell 126:663-676 (2006); Okita. et al., Nature 448:313-317 (2007); Nakagawa et al., Nat. Biotechnol. 26:101-106 (2007); Takahashi et al., Cell 131:1-12 (2007); Meissner et al. Nat. Biotech. 25:1177-1181 (2007); Yu et al. Science 318:1917-1920 (2007); Park et al. Nature 451:141-146 (2008); and U.S. Patent Application Publication No. 2008/0233610, which are hereby incorporated by reference in their entirety). Other methods for generating IPS cells include those disclosed in WO2007/069666, WO2009/006930, WO2009/006997, WO2009/007852, WO2008/118820, U.S. Patent Application Publication No. 2011/0200568 to Ikeda et al., U.S. Patent Application Publication No 2010/0156778 to Egusa et al., U.S. Patent Application Publication No 2012/0276070 to Musick, and U.S. Patent Application Publication No 2012/0276636 to Nakagawa, Shi et al., Cell Stem Cell 3(5):568-574 (2008), Kim et al., Nature 454:646-650 (2008), Kim et al., Cell 136(3):411-419 (2009), Huangfu et al., Nat. Biotechnol. 26:1269-1275 (2008), Zhao et al., Cell Stem Cell 3:475-479 (2008), Feng et al., Nat. Cell Biol. 11:197-203 (2009), and Hanna et al., Cell 133(2):250-264 (2008) which are hereby incorporated by reference in their entirety.

The methods of iPSC generation described above can be modified to include small molecules that enhance reprogramming efficiency or even substitute for a reprogramming factor. These small molecules include, without limitation, epigenetic modulators such as, the DNA methyltransferase inhibitor 5′-azacytidine, the histone deacetylase inhibitor VPA, and the G9a histone methyltransferase inhibitor BIX-01294 together with BayK8644, an L-type calcium channel agonist. Other small molecule reprogramming factors include those that target signal transduction pathways, such as TGF-β inhibitors and kinase inhibitors (e.g., kenpaullone) (see review by Sommer and Mostoslaysky, “Experimental Approaches for the Generation of Induced Pluripotent Stem Cells,” Stem Cell Res. Ther. 1:26 doi:10.1186/scrt26 (Aug. 10, 2010), which is hereby incorporated by reference in its entirety).

Methods of obtaining highly enriched preparations of glial progenitor cells from the iPSCs that are suitable for the methods described herein are disclosed in WO2014/124087 to Goldman and Wang, and Wang et al., “Human iPSC-Derived Oligodendrocyte Progenitors Can Myelinate and Rescue a Mouse Model of Congenital Hypomyelination,” Cell Stem Cell 12(2):252-264 (2013), which are hereby incorporated by reference in their entirety.

In another embodiment, the human glial progenitor cells are derived from embryonic stem cells. Human embryonic stem cells provide a virtually unlimited source of clonal/genetically modified cells potentially useful for tissue replacement therapies. Methods of obtaining highly enriched preparations of glial progenitor cells from embryonic cells that are suitable for use in the methods of the present disclosure are described in Wang et al., “Human iPSC-derived oligodendrocyte progenitor cells can myelinate and rescue a mouse model of congenital hypomyelination,” Cell Stem Cell 12:252-264 (2013), which is hereby incorporated by reference in its entirety.

In another embodiment, the human glial progenitor cells are derived from human fetal tissue. Glial progenitor cells can be extracted from fetal brain tissue containing a mixed population of cells directly by using the promoter specific separation technique as described in U.S. Patent Application Publication Nos. 20040029269 and 20030223972 to Goldman, which are hereby incorporated by reference in their entirety. This method involves selecting a promoter which functions specifically in glial progenitor cells, and introducing a nucleic acid encoding a marker protein under the control of said promoter into the mixed population cells. The mixed population of cells is allowed to express the marker protein and the cells expressing the marker protein are separated from the population of cells, with the separated cells being the glial progenitor cells. Human glial progenitor cells can be isolated from ventricular or subventricular zones of the brain or from the subcortical white matter.

Glial specific promoters that can be used for isolating glial progenitor cells from a mixed population of cells include the CNP promoter (Scherer et al., Neuron 12:1363-75 (1994), which is hereby incorporated by reference in its entirety), an NCAM promoter (Hoist et al., J. Biol. Chem. 269:22245-52 (1994), which is hereby incorporated by reference in its entirety), a myelin basic protein promoter (Wrabetz et al., J. Neurosci. Res. 36:455-71 (1993), which is hereby incorporated by reference in its entirety), a JC virus minimal core promoter (Krebs et al., J. Virol. 69:2434-42 (1995), which is hereby incorporated by reference in its entirety), a myelin-associated glycoprotein promoter (Laszkiewicz et al., “Structural Characterization of Myelin-associated Glycoprotein Gene Core Promoter,” J. Neurosci. Res. 50(6): 928-36 (1997), which is hereby incorporated by reference in its entirety), or a proteolipid protein promoter (Cook et al., “Regulation of Rodent Myelin Proteolipid Protein Gene Expression,” Neurosci. Lett. 137(1): 56-60 (1992); Wight et al., “Regulation of Murine Myelin Proteolipid Protein Gene Expression,” J. Neurosci. Res. 50(6): 917-27 (1997); and Cambi et al., Neurochem. Res. 19:1055-60 (1994), which are hereby incorporated by reference in their entirety). See also U.S. Pat. No. 6,245,564 to Goldman et al., which is hereby incorporated by reference in its entirety.

The glial progenitor cell population derived from fetal tissue can be enriched for by first removing neurons or neural progenitor cells from the mixed cell population. Where neuronal progenitor cells are to be separated from the mixed population of cells, they can be removed based on their surface expression of NCAM, PSA-NCAM, or any other surface moiety specific to neurons or neural progenitor cells. Neurons or neural progenitor cells may also be separated from a mixed population of cells using the promoter based separation technique. Neuron or neural progenitor specific promoters that can be used for separating neural cells from a mixed population of cells include the Tal tubulin promoter (Gloster et al., J. Neurosci. 14:7319-30 (1994) which is hereby incorporated by reference in its entirety), a Hu promoter (Park et al., “Analysis of Upstream Elements in the HuC Promoter Leads to the Establishment of Transgenic Zebrafish with Fluorescent Neurons,” Dev. Biol. 227(2): 279-93 (2000), which is hereby incorporated by reference in its entirety), an ELAV promoter (Yao et al., “Neural Specificity of ELAV Expression: Defining a Drosophila Promoter for Directing Expression to the Nervous System,” J. Neurochem. 63(1): 41-51 (1994), which is hereby incorporated by reference in its entirety), a MAP-1B promoter (Liu et al., Gene 171:307-08 (1996), which is hereby incorporated by reference in its entirety), or a GAP-43 promoter. Techniques for introducing the nucleic acid molecules of the construct into the plurality of cells and then sorting the cells are described in U.S. Pat. No. 6,245,564 to Goldman et al., and U.S. Patent Application Publication No. 20040029269 to Goldman et al., which are hereby incorporated by reference in their entirety.

As an alternative to using promoter-based cell sorting to recover glial progenitor cells from a mixed population of cells, an immunoseparation procedure can be utilized. In a positive immunoseparation technique, the desired cells (i.e. glial progenitor cells) are isolated based on proteinaceous surface markers naturally present on the progenitor cells. For example, the surface marker A2B5 is an initially expressed early marker of glial progenitor cells (Nunes et al., “Identification and Isolation of Multipotential Neural Progenitor Cells from the Adult Human White Matter,” Soc. Neurosci. Abstr. (2001), which is hereby incorporated by reference in its entirety). Using an antibody specific to A2B5, glial progenitor cells can be separated from a mixed population of cell types. Similarly, the surface marker CD44 identifies astrocyte-biased glial progenitor cells (Liu et al., “CD44 Expression Identifies Astrocyte-Restricted Precursor Cells,” Dev. Biol. 276:31-46 (2004), which is hereby incorporated by reference in its entirety). Using CD44-conjugated microbead technology, astroctye-biased glial progenitor cells can be separated from a mixed population of cell types. Oligodendrocyte-biased glial progenitor cells can be separated from a mixed population of cell types based on expression of PDGFαR, the PDGFαR ectodomain CD140a, or CD9. Cells expressing markers of non-glial cell types (e.g., neurons, inflammatory cells, etc.) can be removed from the preparation of glial cells to further enrich the preparation for the desired glial cell type using immunoseparation techniques. For example, the glial progenitor cell population is preferably negative for a PSA-NCAM marker and/or other markers for cells of neuronal lineage, negative for one or more inflammatory cell markers, e.g., negative for a CD11 marker, negative for a CD32 marker, and/or negative for a CD36 marker, which are markers for microglia. Exemplary microbead technologies include MACS® Microbeads, MACS® Columns, and MACS® Separators. Additional examples of immunoseparation are described in Wang et al., “Prospective Identification, Direct Isolation, and Expression Profiling of a Telomerase Expressing Subpopulation of Human Neural Stem Cells, Using Sox2 Enhancer-Directed FACS,” J. Neurosci. 30:14635-14648 (2010); Keyoung et al., “High-Yield Selection and Extraction of Two Promoter-Defined Phenotypes of Neural Stem Cells from the Fetal Human Brain,” Nat. Biotechnol. 19:843-850 (2001); and Windrem et al., “Neonatal Chimerization with Human Glial Progenitor Cells can both Remyelinate and Rescue the Otherwise Lethally Hypomyelinated Shiverer Mouse,” Cell Stem Cell 2:553-565 (2008), which are hereby incorporated by reference in their entirety.

In accordance with the methods described herein, the selected preparation of administered human glial progenitor cells comprise at least about 80% glial progenitor cells, including, for example, about 80%, 85%, 90%, 95%, 96%, 97%, 98%, 99%, 100% glial progenitor cells. The selected preparation of glial progenitor cells can be relatively devoid (e.g., containing less than 20, 15, 10, 9, 8, 7, 6, 5, 4, 3, 2, or 1%) of other cells types such as neurons or cells of neuronal lineage, fibrous astrocytes and cells of fibrous astrocyte lineage, and pluripotential stem cells (like ES cells). Optionally, example cell populations are substantially pure populations of glial progenitor cells.

The glial progenitor cells of the administered preparation can optionally be genetically modified to express other proteins of interest. For example, the glial progenitor cells may be modified to express a therapeutic biological molecule, an exogenous targeting moiety, an exogenous marker (for example, for imaging purposes), or the like. The glial progenitor cells of the preparations can be optionally modified to overexpress an endogenous biological molecule, targeting moiety, and/or marker.

The glial progenitor cells of the administered preparation may be astrocyte-biased glial progenitor cells, oligodendrocyte-biased glial progenitor cells, unbiased glial progenitor cells, or a combination thereof. The glial progenitor cells of the administered preparation express one or more markers of the glial cell lineage. For example, in one embodiment, the glial progenitor cells of the administered preparation may express A2B5+. In another embodiment, glial progenitor cells of the administered preparation are positive for a PDGFαR marker. The PDGFαR marker is optionally a PDGFαR ectodomain, such as CD140a. PDGFαR and CD140a are markers of an oligodendrocyte-biased glial progenitor cells. In another embodiment, glial progenitor cells of the administered preparation are CD44+. CD44 is a marker of an astrocyte-biased glial progenitor cell. In another embodiment, glial progenitor cells of the administered preparation are positive for a CD9 marker. The CD9 marker is optionally a CD9 ectodomain. In one embodiment, the glial progenitor cells of the preparation are A2B5+, CD140a+, and/or CD44+. The aforementioned glial progenitor cell surface markers can be used to identify, separate, and/or enrich the preparation for glial progenitor cells prior to administration.

The administered glial progenitor cell preparation is optionally negative for a PSA-NCAM marker and/or other neuronal lineage markers, and/or negative for one or more inflammatory cell markers, e.g., negative for a CD11 marker, negative for a CD32 marker, and/or negative for a CD36 marker (which are markers for microglia). Optionally, the preparation of glial progenitor cells are negative for any combination or subset of these additional markers. Thus, for example, the preparation of glial progenitor cells is negative for any one, two, three, or four of these additional markers.

Suitable methods of introducing cells into the striatum, forebrain, brain stem, and/or cerebellum of a subject are well known to those of skill in the art and include, but are not limited to, injection, deposition, and grafting as described herein.

In one embodiment, the glial progenitor cells are transplanted bilaterally into multiple sites of the subject as described U.S. Pat. No. 7,524,491 to Goldman, Windrem et al., “Neonatal Chimerization With Human Glial Progenitor Cells Can Both Remyelinate and Rescue the Otherwise Lethally Hypomyelinated Shiverer Mouse,” Cell Stem Cell 2:553-565 (2008), Han et al., “Forebrain Engraftment by Human Glial Progenitor Cells Enhances Synaptic Plasticity and Learning Adult Mice,” Cell Stem Cell 12:342-353 (2013), and Wang et al., “Human iPSCs-Derived Oligodendrocyte Progenitor Cells Can Myelinate and Rescue a Mouse Model of Congenital Hypomyelination,” Cell Stem Cell 12:252-264 (2013), which are hereby incorporated by reference in their entirety). Methods for transplanting nerve tissues and cells into host brains are described by Bjorklund and Stenevi (eds), Neural Grafting in the Mammalian CNS, Ch. 3-8, Elsevier, Amsterdam (1985); U.S. Pat. No. 5,082,670 to Gage et al.; and U.S. Pat. No. 6,497,872 to Weiss et al., which are hereby incorporated by reference in their entirety. Typical procedures include intraparenchymal, intracallosal, intraventricular, intrathecal, and intravenous transplantation.

Intraparenchymal transplantation is achieved by injection or deposition of tissue within the host brain so as to be apposed to the brain parenchyma at the time of transplantation. The two main procedures for intraparenchymal transplantation are: 1) injecting the donor cells within the host brain parenchyma or 2) preparing a cavity by surgical means to expose the host brain parenchyma and then depositing the graft into the cavity (Bjorklund and Stenevi (eds), Neural Grafting in the Mammalian CNS, Ch. 3, Elsevier, Amsterdam (1985), which is hereby incorporated by reference in its entirety). Both methods provide parenchymal apposition between the donor cells and host brain tissue at the time of grafting, and both facilitate anatomical integration between the graft and host brain tissue. This is of importance if it is required that the donor cells become an integral part of the host brain and survive for the life of the host.

Glial progenitor cells can also be delivered intracallosally as described in U.S. Patent Application Publication No. 20030223972 to Goldman, which is hereby incorporated by reference in its entirety. The glial progenitor cells can also be delivered directly to the forebrain subcortex, specifically into the anterior and posterior anlagen of the corpus callosum. Glial progenitor cells can also be delivered to the cerebellar peduncle white matter to gain access to the major cerebellar and brainstem tracts. Glial progenitor cells can also be delivered to the spinal cord.

Alternatively, the cells may be placed in a ventricle, e.g., a cerebral ventricle. Grafting cells in the ventricle may be accomplished by injection of the donor cells or by growing the cells in a substrate such as 30% collagen to form a plug of solid tissue which may then be implanted into the ventricle to prevent dislocation of the graft cells. For subdural grafting, the cells may be injected around the surface of the brain after making a slit in the dura.

Suitable techniques for glial cell delivery are described supra. In one embodiment, said preparation of glial progenitor cells is administered to one or more sites of the brain, brain stem, spinal cord, or combinations thereof.

Delivery of the cells to the subject can include either a single step or a multiple step injection directly into the nervous system. Although adult and fetal oligodendrocyte precursor cells disperse widely within a transplant recipient's brain, for widespread disorders, multiple injections sites can be performed to optimize treatment. Injection is optionally directed into areas of the central nervous system such as white matter tracts like the corpus callosum (e.g., into the anterior and posterior anlagen), dorsal columns, cerebellar peduncles, cerebral peduncles. Such injections can be made unilaterally or bilaterally using precise localization methods such as stereotaxic surgery, optionally with accompanying imaging methods (e.g., high resolution MRI imaging). One of skill in the art recognizes that brain regions vary across species; however, one of skill in the art also recognizes comparable brain regions across mammalian species.

The cellular transplants are optionally injected as dissociated cells but can also be provided by local placement of non-dissociated cells. In either case, the cellular transplants optionally comprise an acceptable solution. Such acceptable solutions include solutions that avoid undesirable biological activities and contamination. Suitable solutions include an appropriate amount of a pharmaceutically-acceptable salt to render the formulation isotonic. Examples of the pharmaceutically-acceptable solutions include, but are not limited to, saline, Ringer's solution, dextrose solution, and culture media. The pH of the solution is preferably from about 5 to about 8, and more preferably from about 7 to about 7.5.

The injection of the dissociated cellular transplant can be a streaming injection made across the entry path, the exit path, or both the entry and exit paths of the injection device (e.g., a cannula, a needle, or a tube). Automation can be used to provide a uniform entry and exit speed and an injection speed and volume.

The number of glial progenitor cells administered to the subject can range from about 10²-10⁸ at each administration (e.g., injection site), depending on the size and species of the recipient, and the volume of tissue requiring cell replacement. Single administration (e.g., injection) doses can span ranges of 10³-10⁵, 10⁴-10⁷, and 10⁵-10⁸ cells, or any amount in total for a transplant recipient patient.

Since the CNS is an immunologically privileged site, administered cells, including xenogeneic, can survive and, optionally, no immunosuppressant drugs or a typical regimen of immunosuppressant agents are used in the treatment methods. However, optionally, an immunosuppressant agent may also be administered to the subject. Immunosuppressant agents and their dosing regimens are known to one of skill in the art and include such agents as Azathioprine, Azathioprine Sodium, Cyclosporine, Daltroban, Gusperimus Trihydrochloride, Sirolimus, and Tacrolimus. Dosages ranges and duration of the regimen can be varied with the disorder being treated; the extent of rejection; the activity of the specific immunosuppressant employed; the age, body weight, general health, sex and diet of the subject; the time of administration; the route of administration; the rate of excretion of the specific immunosuppressant employed; the duration and frequency of the treatment; and drugs used in combination. One of skill in the art can determine acceptable dosages for and duration of immunosuppression. The dosage regimen can be adjusted by the individual physician in the event of any contraindications or change in the subject's status.

As described above, another aspect of the present disclosure relates to a method of increasing oligodendrocyte production from human glial progenitor cells. This method involve providing a population of human glial progenitor cells and administering in vitro to the population of human glial progenitor cells one or modulators of one or more cell signaling pathways described above, under conditions effective increase oligodendrocyte production compared to oligodendrocyte production in the absence of administration of the one or more modulators.

Human glial progenitor cells and methods of obtaining human glial progenitor cells are described supra.

Modulators of one or more genes as described in Table 3 are also described supra as well embodiments comprising specific cell signaling pathways.

### EXAMPLES

The examples below are intended to exemplify the practice of embodiments of the disclosure but are by no means intended to limit the scope thereof.

### Materials and Methods for Examples

Cells. Human glial progenitor cells (hGPCs) were sorted from 18-22 week g.a. human fetuses, obtained from the surgical pathology suite, by either A2B5- or CD140a-directed isolation. Acquisition, dissociation and immunomagnetic sorting of A2B5+/PSA-NCAM− cells were as described (Windrem et al., “Fetal and Adult Human Oligodendrocyte Progenitor Cell Isolates Myelinate the Congenitally Dysmyelinated Brain,” Nat Med 10:93-97 (2004), which is hereby incorporated by reference in its entirety). GPCs were isolated from dissociated tissue using a dual immunomagnetic sorting strategy: depleting mouse anti-PSA-NCAM+ (Millipore, DSHB) cells, using microbead tagged rat anti-mouse IgM (Miltenyi Biotech), then selecting A2B5+(clone 105; ATCC, Manassas, Va.) cells from the PSA-NCAM-pool, as described (Windrem et al., “Fetal and Adult Human Oligodendrocyte Progenitor Cell Isolates Myelinate the Congenitally Dysmyelinated Brain,” Nat Med 10:93-97 (2004); Windrem et al., “Neonatal Chimerization With Human Glial Progenitor Cells Can Both Remyelinate and Rescue the Otherwise Lethally Hypomyelinated Shiverer Mouse,” Cell Stem Cell 2:553-565 (2008), which are hereby incorporated by reference in their entirety). After sorting, cells were maintained for 1-14 days in DMEM-F12/N1 with 10 ng/ml bFGF and 20 ng/ml PDGF-AA. Alternatively for some experiments, CD140a/PDGFαR-defined GPCs were isolated and sorted using MACS as described (Sim et al., “CD140a Identifies a Population of Highly Myelinogenic, Migration-Competent and Efficiently Engrafting Human Oligodendrocyte Progenitor Cells,” Nature Biotechnology 29:934-941 (2011), which is hereby incorporated by reference in its entirety), yielding an enriched population of CD140+glial progenitor cells.

Animal Models and Transplantation.

Shiverer mice, engrafted as adults. Homozygous Shi^{-/-}×rag2^{-/-}, rag2^{-/-}, and rag1^{-/-} immunodeficient mice were bred and housed in a pathogen-free environment in accordance with University of Rochester animal welfare regulations. Mice from each genotype were transplanted between the ages of 4-12 weeks with 1×10⁵ hGPCs/1 μl/hemisphere (n=2-4 mice/time point/genotype), delivered bilaterally to the genu of the corpus callosum at coordinates: AP −0.8; ML ±0.75; DV −1.25, all relative to bregma. Mice were injected with FK506 (5 mg/kg, i.p.; Tecoland, Inc.) daily for 3 days pre- and 3 days post-surgery. All shi^{-/-}×rag2^{-/-} mice were killed at age 19-20 weeks, or when clinical morbidity, as defined in the animal welfare policy, was observed. For the myelin wildtype mice, half of all rag2^{-/-} and rag1^{-/-} animals were sacrificed between 20-22 weeks of age, and the other half at 1 year.

Myelin wild-type mice, neonatally transplanted, cuprizone-demyelinated as adults. Homozygous rag1-null immunodeficient (rag1^{-/-}) mice on a C57BL/6 background were bred in the colony. Animals were transplanted with hGPCs neonatally, via bilateral injections delivered to the presumptive corpus callosum (Windrem et al., “Fetal and Adult Human Oligodendrocyte Progenitor Cell Isolates Myelinate the Congenitally Dysmyelinated Brain,” Nat Med 10:93-97 (2004), which is hereby incorporated by reference in its entirety), so as to engraft newborn recipient brains before cuprizone demyelination. Beginning at 17 weeks of age, these mice were fed ad libitum a diet containing 0.2% (w/w) cuprizone (S5891, BioServe) for 12 weeks and then returned to normal diet. Littermate and non-littermate controls were maintained on a normal diet. Mice were sacrificed before diet (17 weeks), during diet (25 weeks), immediately after diet completion (29 weeks), and after either 8 weeks (37 weeks old) or 20 weeks (49 weeks old) of post-cuprizone recovery.

Myelin wild-type mice, cuprizone-demyelinated as adults, then transplanted. Homozygous rag1-null mice were subjected to cuprizone demyelination as noted, for a 20-week period beginning at 6 weeks of age. They were transplanted with hGPCs at 10 weeks of age, 4 weeks into their period of cuprizone demyelination. At that point, the mice were transplanted with a total of 200,000 PSA-NCAM−/A2B5+ cells, delivered sterotaxically as 1×10⁵ hGPCs/1 μl HMS into the corpus callosum bilaterally at the following coordinates: from bregma, AP −0.8 mm, ML ±0.75; from dura, DV −1.25 mm. Upon recovery, mice were returned to their cages. Mice were injected with FK506 (5 mg/kg, i.p.; Tecoland, Inc.) daily for 3 days pre- and 3 days post-surgery. Mice were sacrificed during diet (18 weeks), immediately after diet completion (26 weeks), and after 20 weeks (46 weeks old) of postcuprizone recovery.

Histology. All mice were perfused with HMS (−) followed by 4% paraformaldehyde. Brains were cryopreserved with 6%, then 30% sucrose and embedded coronally in OCT (TissueTek). Brains were then cut at 20 μm on a Leica cryostat. Sections were processed for one or more of the antigenic markers (see Table 4 below).

Quantification. The optical fractionator method was used to quantify the phenotype of cells in the corpus callosum for: oligodendrocytes (transferrin), astrocytes (GFAP), and progenitors (PDGFRα). Transferrin, a cytoplasmic and membrane-localized iron transport protein, permits identification of colabeling with human nuclear antigen for the purpose of quantification by species of origin (Connor et al., “Development of Transferrin-Positive Oligodendrocytes in the Rat Central Nervous System,” Journal of Neuroscience Research 17:51-59(1987); Connor et al., “Transferrin in the Central Nervous System of the Shiverer Mouse Myelin Mutant,” Journal of Neuroscience Research 36:501-507 (1993), which are hereby incorporated by reference in their entirety). Quantification of the phenotypes in the corpus callosum was performed by using a computerized stereology system consisting of a BX-51 microscope (Olympus) equipped with a Ludl (Hawthorne, N.Y.) XYZ motorized stage, Heidenhain (Plymouth, Minn.) z-axis encoder, an Optronics (East Muskogee, Okla.) QuantiFire black and white video camera, a Dell (Round Rock, Tex.) PC workstation, and Stereo Investigator software (MicroBrightField, Wiliston, Vt.). Within each corpus callosum, beginning at a random starting point where it crosses the midline, 3 sections equidistantly spaced 480 μm apart were selected for analysis. The corpus callosum was outlined from the midline to 1 mm lateral, and all cells were counted. Upper and lower exclusion zones of 10% of section thickness were used.

RNA-Sequencing and analysis of FACS isolated hGPCs. To observe transcriptional changes in hGPCs following demyelination neonatal rag1^{-/-} mice were transplanted with 2×10⁵ hGPCs as described above. At 12 weeks, mice either maintained a normal diet or were transitioned to a 0.2% (w/w) cuprizone diet fed ad libitum until 24 weeks of age when they were returned to a normal diet. At 36 weeks, all mice were anesthetized with sodium pentobarbital, perfused with ice cold HBSS^{+/+} (containing calcium and magnesium) (Thermo Fisher), and the brains extracted and placed into fresh HBSS^{+/+} in a tissue culture plate. Excess HBSS^{+/+} was removed and the corpus callosum of each animal was isolated surgically, minced, and suspended in HBSS^{-/-}. Corpora callosa from two mice from the same experimental group were pooled for each study. The tissue was transferred to a 15 ml conical tube and rinsed twice with HBSS^{-/-}. The tube was filled to maximum volume with HBSS^{-/-}, and then centrifuged for 5 minutes at 1,500 rpm. The supernatant was then removed and 0.62 units Research Grade Liberase DH (Roche) was added, and the samples then incubated at 37° C. for 40 minutes with gentle rocking. Following Liberase dissociation, 2 ml 0.5% BSA in MEM (Thermo Fisher) supplemented with 500 units bovine pancreas DNase (Sigma) and 0.5 ml PD-FBS (Cocalico Biologicals) was added to the sample. The sample was triturated with a p1000 Pipetman and then passed through a 70 μm cell strainer. MEM containing 0.5% BSA was then added to the tube to bring it to full volume, and the tube centrifuged at 1,500 rpm for 10 minutes. Dissociated cells were then tagged with anti-CD140a-PE and sorted via FACS as previously reported (Sim et al., “CD140a Identifies a Population of Highly Myelinogenic, Migration-Competent and Efficiently Engrafting Human Oligodendrocyte Progenitor Cells,” Nature Biotechnology 29:934-941 (2011), which is hereby incorporated by reference in its entirety). Cells were lysed and prepared for library construction via Prelude Direct Lysis Module (NuGEN) according to the manufacturer's protocol.

Libraries were constructed using Ovation RNA-Seq System V2 (NuGEN) according to manufacturer's protocol and sequenced with a read length of paired-end 125 bp on a HiSeq 2500 system (Illumina). Reads were demultiplexed and cleaned using Trimmomatic (Bolger et al., “Trimmomatic: A Flexible Trimmer for Illumina Sequence Data,” Bioinformatics 30:2114-2120 (2014), which is hereby incorporated by reference in its entirety). Reads were aligned to human genome GRCh38,p10 and mapped to Ensembl reference 91 via STAR 2.5.2b (Dobin et al., “STAR Ultrafast Universal RNA-seq Aligner,” Bioinformatics 29(1):15-21 (2013), which is hereby incorporated by reference in its entirety), with quantMode set to TranscriptomeSAM. Gene abundances and expected counts were then calculated using RSEM 1.3.0 (Li et al., “RSEM: Accurate Transcript Quantification from RNA-Seq Data With or Without a Reference Genome,” BMC Bioinformatics 12:323 (2011), which is hereby incorporated by reference in its entirety). Expected counts were imported into R via tximport for differential expression analysis between cuprizone and control hGPCs (R Core Team, “R: A Language and Environment for Statistical Computing. (Vienna, Austria: R Foundation for Statistical Computing),” Open Journal of Statistics 7(5) (2017); Soneson et al., “Differential Analyses for RNA-Seq: Transcript-Level Estimates Improve Gene-Level Inferences,” F1000Research 4:1521 (2015), which are hereby incorporated by reference in their entirety). Low-expressing genes were removed prior to analysis if their expected counts fell below a median of 3 in both conditions. Full within-lane normalization of samples was conducted using EDASeq to adjust for GC-content effects prior to the generation of library size adjusted counts via DESeq2 (Love et al., “Moderated Estimation of Fold Change and Dispersion for RNA-Seq Data with DESeq2,” Genome Biology 15:550 (2014); Risso et al., “GC-Content Normalization for RNA-Seq Data,” BMC Bioinformatics 12:480 (2011), which are hereby incorporated by reference in their entirety). Differential expression between post-cuprizone and control hGPCs was then determined using DESeq2 following the addition of a variance factor to the generalized linear model. This factor was calculated using RUVSeq's RUVs function with all genes set as in silico negative controls (Risso et al., “Normalization of RNA-Seq Data Using Factor Analysis of Control Genes or Samples,” Nature Biotechnology 32:896-902 (2014), which is hereby incorporated by reference in its entirety). Genes with an adjusted p-value <0.05 were considered significant. Only genes with mean transcripts per million (TPM) >6.5 in either group were kept for functional analysis, as they were more likely to be biologically significant.

For functional analysis and inference of gene interactivity, a gene ontology network was constructed. Differentially expressed genes between both groups were analyzed in Ingenuity Pathway Analysis (QIAGEN) where 43 significantly enriched terms were selected based on relevance, along with their contributing differentially expressed genes, to be used as nodes. Along with the undirected edges derived from gene-GO term associations, edges were further generated via IPA's curated database connecting genes with known interactions. Network visualization was carried out in Cytoscape (Shannon, P., “Cytoscape: A Software Environment for Integrated Models of Biomolecular Interaction Networks,” Genome Res 13:2498-2504 (2003), which is hereby incorporated by reference in its entirety) with the determination of modularity occurring in Gephi (Bastian et al., “Gephi: An Open Source Software for Exploring and Manipulating Networks,” Proceedings of the Third International ICWSM Conference 361-362 (2009), which is hereby incorporated by reference in its entirety). Nodes were clustered within their respective modules and aesthetically repositioned slightly. Gene expression data are available via GEO, accession number GSE112557.

Antibodies. Phenotyping of donor cells was accomplished by immunostaining for human nuclear antigen (Millipore, clone MAB1281), together with one or more of the following:

TABLE 4

| | Antigen | Name | dilution | Catalog | Company | RRID |
|---|---|---|---|---|---|---|
| In vivo | hGFAP | Mouse anti- | 1:500 | SMI-21R | Covance | AB_509979 |
| | | human glial | | | | |
| | | fibrillary acidic | | | | |
| | | protein | | | | |
| | hN | Mouse anti- | 1:800 | MAB1281 | Millipore | AB_94090 |
| | | human nuclei, | | | | |
| | | clone 235-1 | | | | |
| | hNG2 | Mouse anti- | 1:200 | MAB2029 | Millipore | AB_94509 |
| | | NG2, clone | | | | |
| | | 9.2.27 | | | | |
| | MBP | Rat anti-Myelin | 1:25 | ab7349 | Abeam | AB_305869 |
| | | Basic Protein | | | | |
| | hPDGFRa | Rabbitanti-human | 1:300 | 5241S | Cell | AB_10692773 |
| | | PDGFRa, clone | | | Signal | |
| | | D1306 | | | | |
| | Transferrin | Transferrin | 1:800 | ab9538 | Abeam | AB_307325 |
| | | antibody | | | | |
| | | (ab9538) | | | | |
| In vitro | PSA- | Mouse anti- | 1:100 | MAB5324 | Millipore | AB_95211 |
| | NCAM | PSA- NCAM, | | | | |
| | | clone 2-2B | | | | |
| | PSA- | Mouse anti-PSA- | 1:1 | 5A5 | DSHB | AB_528392 |
| | NCAM | NCAM | | | | |
| | | supernatant, 5A5 | | | | |
| | A2B5 | Mouse anti-A2B5 | 1:1 | CRL-1520 | ATCC | CVCL_7946 |
| | | supernatant, clone | | | | |
| | | 105 | | | | |
| | CD140 | Mouse anti- | 0.21 μg/10⁶ cells | 556001 | BD | AB 396285 |
| | | CD140a, Clone | | | Biosci. | |
| | | AR1 | | | | |
| | CD140a- | PE-conj mouse | 10 μL/10⁶ cells | 556002 | BD Biosci | AB_396286 |
| | PE | anti- human | | | | |
| | | CD140a, AR1 | | | | |
| Secondary | | Rat anti-Mouse | 20 μL/10⁷ cells | 130-047- | Miltenyi | AB 244358 |
| | | IgM | | 301 | | |
| | | MicroBeads | | | | |
| | | Rat anti-Mouse | 10 μL/10⁷ cells | 130-047- | Miltenyi | AB 244356 |
| | | IgG2a + b | | 201 | | |
| | | MicroBeads | | | | |
| | | AlexaFluor 568 | 1:400 | A-11031 | Invitrogen | AB_144696 |
| | | Goatanti-Mouse | | | | |
| | | IgG (H + L) | | | | |
| | | AlexaFluor 568 | 1:400 | A-21124 | Invitrogen | AB_2535766 |
| | | Goat anti-Mouse | | | | |
| | | IgG1 | | | | |
| | | AlexaFluor 488 | 1:400 | A-11029 | Invitrogen | AB_2534088 |
| | | Goat anti-Mouse | | | | |
| | | IgG (H + L) | | | | |
| | | AlexaFluor 488 | 1:400 | A-21121 | Invitrogen | AB_2535764 |
| | | Goat anti-Mouse | | | | |
| | | IgG1 | | | | |
| | | AlexaFluor 647 | 1:400 | A-21235 | Jackson | AB_2535813 |
| | | Goat anti-Mouse | | | | |
| | | IgG (H + L) | | | | |
| | | AlexaFluor 568 | 1:400 | A-11036 | Invitrogen | AB_2534094 |
| | | Goat anti-Rabbit | | | | |
| | | IgG (H + L) | | | | |
| | | AlexaFluor 488 | 1:400 | A-11034 | Invitrogen | AB_2576217 |
| | | Goat anti-Rabbit | | | | |
| | | IgG (H + L) | | | | |
| | | AlexaFluor 568 | 1:400 | A-11077 | Invitrogen | AB_2534121 |
| | | Goat anti-Rat | | | | |
| | | IgG (H + L) | | | | |
| | | AlexaFluor 488 | 1:400 | A-11006 | Invitrogen | AB_2534074 |
| | | Goat anti-Rat | | | | |
| | | IgG (H + L) | | | | |

### Example 1—Adult Shiverer Mice Exhibit Myelination Following hGPC Delivery

To assess the ability of donor hGPCs to disperse and differentiate as oligodendroglia in the adult brain, CD140a-sorted fetal hGPCs were introduced into young adult shiverer×rag2^{-/-} immunedeficient mice (Sim et al., “CD140a Identifies a Population of Highly Myelinogenic, Migration-Competent and Efficiently Engrafting Human Oligodendrocyte Progenitor Cells,” Nature Biotechnology 29:934-941 (2011), which is hereby incorporated by reference in its entirety), as well as into two normally myelinated immunodeficient control lines, rag1^{-/-} on a C57Bl/6 background, and rag2^{-/-} on C3H. All mice were injected after weaning, over the range of 4-12 weeks of age; the shiverers were all injected between 4-6 weeks. A total of 22 mice (8 shiverers, 14 normally myelinated rag-null mice, both rag1^{-/-} and rag2^{-/-}) were injected bilaterally in both the anterior and posterior corpus callosum, with 2 injections per hemisphere of 5×10⁴ hGPCs each. All 8 shiverers and 6 of the controls were sacrificed 12-15 weeks later at 19-22 weeks of age, while the remaining 8 control mice were sacrificed at approximately 1 year of age. The brains of all mice were examined for donor cell dispersal and oligodendrocytic differentiation as well as for MBP immunoreactivity, which was necessarily donor-derived in the shiverer context.

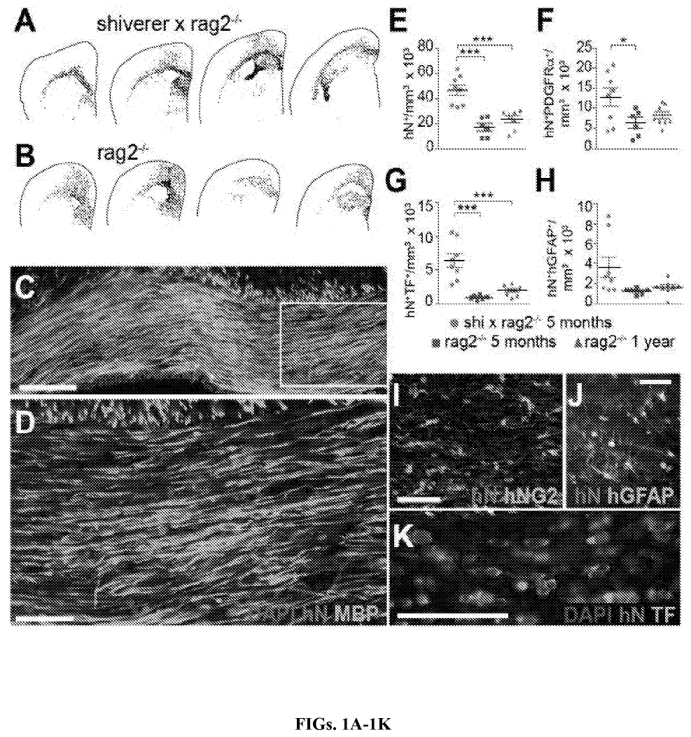

The hGPCs proved both highly migratory and robustly myelinogenic in the adult brain. By 12-15 weeks after transplant, the injected cells had dispersed broadly throughout the forebrain, as is typically observed in similarly-treated neonates (Windrem et al., “Neonatal Chimerization with Human Glial Progenitor Cells can both Remyelinate and Rescue the Otherwise Lethally Hypomyelinated Shiverer Mouse,” Cell Stem Cell 2:553-565 (2008), which is hereby incorporated by reference in its entirety), with a near-uniform distribution of donor cells noted throughout the white matter in both congenitally dysmyelinated shiverers (FIG. 1A) and normally myelinated (FIG. 1B) mice. Myelinogenesis was robust in the shiverers, with dense myelination of the corpus callosum (FIGS. 1C-1D). Importantly, at the 19-week time-point assessed, the callosal densities of all human cells, as well as human GPCs, oligodendrocytes, and astrocytes, were all significantly and substantially higher in the recipient shiverer brains than in their myelin wild-type controls (FIGS. 1E-1H; 11-1K), indicating the overwhelmingly competitive advantage of the human donor cells in the shiverer environment. In myelin wild-type control brains, whether examined at either 5 or 12 months, these cells also expanded and engrafted, but largely remained as progenitors (FIG. 1F). These data indicated that CD140a-sorted hGPCs are able to migrate broadly throughout the young adult mouse brain, that the dispersal of these cells is not impeded by adult brain parenchyma, and that robust myelination of still-viable axons can begin even after a several months' absence of mature myelin in the affected brain.

### Example 2—Resident Human GPCs can Remyelinate the Cuprizone-Demyelinated Corpus Callosum

Cuprizone is a well-studied copper chelator, the chronic oral administration of which causes mitochondrial dysfunction that is both earliest and most prominent in myelinating oligodendrocytes (Morell et al., “Gene Expression in Brain During Cuprizone-Induced Demyelination and Remyelination,” Mol Cell Neurosci 12:220-227 (1998), which is hereby incorporated by reference in its entirety). Its oral administration results in diffuse, relatively synchronous demyelination, which has been well-characterized in a variety of mouse strains and ages (Stidworthy et al., “Quantifying the Early Stages of Remyelination Following Cuprizone-Induced Demyelination,” Brain Pathol 13:329-339 (2003), which is hereby incorporated by reference in its entirety). Cuprizone-induced demyelination is more reproducible than any other current model of demyelination, has little systemic toxicity at demyelinating doses, is associated with little acute axonal injury or neuronal loss, and is relatively non-inflammatory, except for local microglial activation (Matsushima et al., “The Neurotoxicant, Cuprizone, as a Model to Study Demyelination and Remyelination in the Central Nervous System,” Brain Pathol 11:107-116 (2001), which is hereby incorporated by reference in its entirety). To assess the ability of human GPCs to remyelinate newly-demyelinated adult axons, dietary cuprizone was used to induce central demyelination, and the responses of both already-resident and later-introduced hGPCs to that myelin loss were followed.

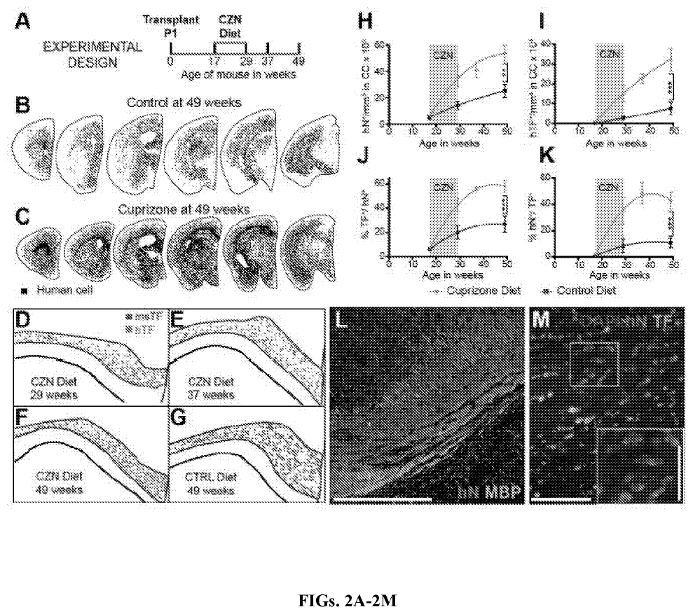

It was first asked if human GPCs already resident within the mouse white matter could differentiate as oligodendrocytes and remyelinate denuded axons after cuprizone challenge. The effects of cuprizone were first assessed on rag1^{-/-}×C57Bl/6 mice, and previous observations were confirmed (Hibbits et al., “Cuprizone Demyelination of the Corpus Callosum in Mice Correlates with Altered Social Interaction and Impaired Bilateral Sensorimotor Coordination,” ASN Neuro 1:e00013 (2009); Hibbits et al., “Astrogliosis During Acute and Chronic Cuprizone Demyelination and Implications for Remyelination,” ASN Neuro 4:393-408 (2012), which are hereby incorporated by reference in their entirety), that a 12-week course of cuprizone induced the widespread loss of transferrin (TF)-defined oligodendrocytes in the corpus callosum, with no detectable loss of resident mouse GPCs. It was then asked if neonatally-implanted human GPCs could similarly tolerate cuprizone exposure, and if so, whether they remained able to generate new oligodendrocytes and remyelinate adult-demyelinated axons. To that end, rag1^{-/-}×C57Bl/6 mice were transplanted on postnatal day 1 with human fetal A2B5+/PSA-NCAM− GPCs, delivered as 10⁵ cells per hemisphere into the corpus callosum bilaterally. This protocol results in widespread colonization of the recipient brains by human GPCs, which ultimately replace many—and typically most—of the host murine GPCs (Windrem et al., 2014). The resultant human glial chimeric mice were then given dietary cuprizone (0.2% w/w) as a food additive, beginning at 4 months of age; by this time, the human NG2+GPCs have largely replaced mouse callosal NG2+ cells (Windrem et al., “A Competitive Advantage by Neonatally Engrafted Human Glial Progenitors Yields Mice Whose Brains are Chimeric for Human Glia,” The Journal of Neuroscience: The Official Journal of the Society for Neuroscience 34:16153-16161 (2014), which is hereby incorporated by reference in its entirety). The experimental mice were left on the cuprizone diet for 12 weeks, while littermate controls were maintained on a normal diet (FIG. 2A). The density of human cells in the host white matter, as well as the percentage of those cells that differentiated as oligodendrocytes, was calculated for both cuprizone-treated and control mice at the start of the dietary manipulation, as well as at each of 3 different time-points: immediately after cuprizone cessation; after 8 weeks' recovery; and after 20 weeks' recovery—the latter out to 1 year of age.

It was found that the human GPCs tolerated cuprizone exposure at least as well as their mouse counterparts, and dispersed broadly throughout the forebrain (FIGS. 2B-2C). The hGPCs then robustly generated new oligodendrocytes and effectively remyelinated the demyelinated white matter after cuprizone cessation (FIGS. 2D-2G; 21-2M). In particular, both the total number of human cells, and the percentage that differentiated as oligodendrocytes, increased significantly faster and to a greater extent in the cuprizone-fed mice than in their matched controls (FIGS. 2D-2G; also 2H-2K). The density of human cells in the corpus callosum of cuprizone-treated mice increased from 5,072±1,611 at 4 months to 53,835±5,898 cells/mm³ at one year; in contrast, over the same period, control mice exhibited a more modest expansion of GPCs, to 25,296±4,959 cells/mm³; p=0.001 by 2-way ANOVA; F=7.40 (FIG. 2H). Importantly, the proportion of all human cells that differentiated as mature oligodendrocytes by 1 year was twice that in the cuprizone-treated mice than in their untreated controls (58.0±4.8% vs. 26.6±6.4%; p<0.0001, F=13.32; FIG. 2J). Similarly, the density of human oligodendrocytes rose over 4.5-fold in the cuprizone-treated mice, from 7,642±3,095 to 32,323±5,850 hTF+ cells/mm³ (p=0.0006; F=8.65; FIG. 2I). These data indicate that cuprizone-induced demyelination yielded a relative increase in both the absolute numbers and relative proportions of parenchymal hGPC-derived oligodendrocytes, and the remyelination of demyelinated host axons by those cells (FIGS. 2L-2M). Thus, those hGPCs already-resident within the callosal white matter responded to acute demyelination by differentiating as mature oligodendrocytes and remyelinating accessible denuded axons. Thus, resident hGPCs could myelinate not only axons that had never been myelinated, as in the adult shiverer brain, but also those that were previously unsheathed by myelin.

### Example 3—Human GPCs can Remyelinate Axons when Delivered after Initial Demyelination

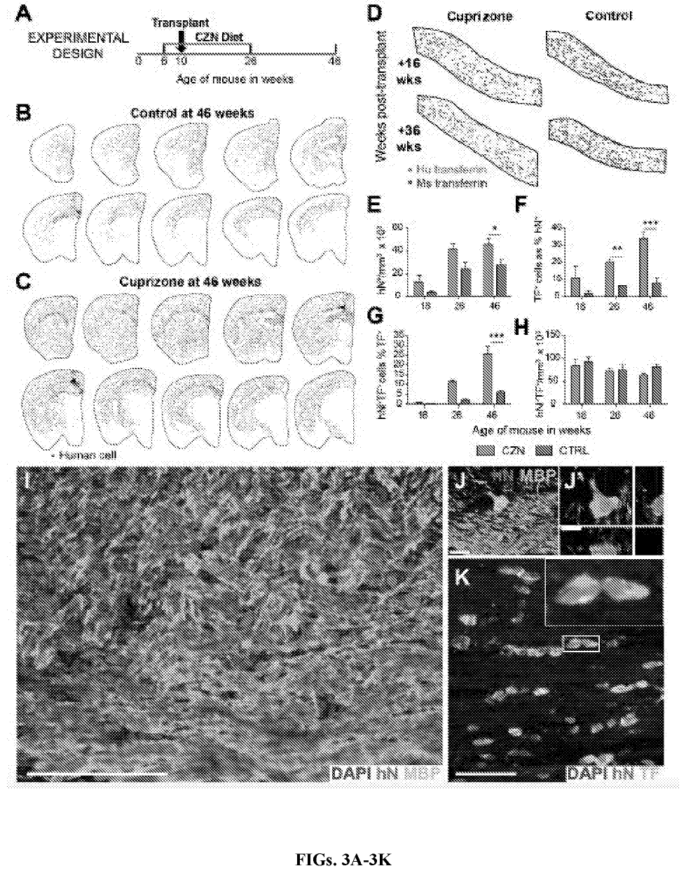

It was next asked if hGPCs delivered to the adult brain after initial demyelination and during ongoing cuprizone exposure, could migrate and myelinate host axons, and whether they were able to myelinate denuded axons as effectively as hGPCs resident in their host brains since neonatal development. To this end, prolonged cuprizone exposure was next used to demyelinate otherwise wildtype adult mice, and hGPCs were transplanted into these demyelinating brains. In particular, to minimize the potential for endogenous remyelination by remaining mouse GPCs, a 20 week cuprizone course was used, which was found to allow much less spontaneous remyelination than shorter periods of cuprizone exposure (FIG. 3A). It was found that even when delivered into adult brain parenchyma 4 weeks after the onset of cuprizone treatment, during active demyelination, that the transplanted hGPCs not only dispersed widely, but did so and expanded more robustly than in untreated control brains (FIGS. 3B-3D; 3E-3F). When the cuprizone fed mice were assessed at 16 weeks after hGPC transplant (26 weeks of age), human oligodendrocytes were apparent, having differentiated from the engrafted hGPCs. By that point, more donor hGPCs had differentiated as oligodendrocytes in the cuprizone-demyelinated brains than in their untreated controls, suggesting both the preferential expansion of hGPCs (FIG. 3E) and the active induction of oligodendrocytic phenotype by the demyelinated environment (FIG. 3F). By 36 weeks post-transplant (46 weeks of age), allowing 20 additional weeks for phenotypic differentiation, over a quarter of all oligodendrocytes in the host white matter were of human origin (FIG. 3G). Remarkably, the overall number and density of transferrin-defined oligodendrocytes, whether of mouse or human origin, was relatively preserved at all time points (FIG. 3H). The transplanted human cells proceeded to robustly differentiate as oligodendrocytes and myelinate the demyelinated tissue, such that by 46 weeks of age—36 weeks post-transplant—much if not most of the forebrain white matter in these previously cuprizone demyelinated brains was of human origin (FIGS. 3I-3K). Thus, human GPCs were able to effectively remyelinate mature axons that had been previously myelinated in the brain, and could do so even when delivered to the adult brain after the onset of demyelination.

### Example 4—Human GPCs Activated Stereotypic Transcriptional Programs after Cuprizone Demyelination

It was next asked whether demyelination and its attendant activation of human GPCs was associated with transcriptional events that might identify early determinants of progenitor cell mobilization, as well as those of astrocytic or oligodendrocytic fate. To thereby identify the responses of human GPCs to demyelination in vivo, human GPCs were isolated from cuprizone-demyelinated, neonatally chimerized brains in which they had been resident, using CD140a-directed fluorescence-activated cell sorting, followed by RNA sequencing. To this end, neonatal rag1^{-/-} mice were transplanted with fetal human hGPCs, and maintained through 12 weeks of age on a normal diet. At that point, control mice were continued on a normal diet, while experimental mice were transitioned to a diet of 0.2% (w/w) cuprizone for 12 weeks, to induce oligodendrocytic death. Cuprizone-demyelinated mice were then allowed to recover for an additional 12 weeks on a normal diet, before both groups were sacrificed at 36 weeks of age. The callosal white matter was then dissected, dissociated, and CD140a+hGPCs isolated via FACS. The RNA of these hGPC isolates was then extracted and sequenced.

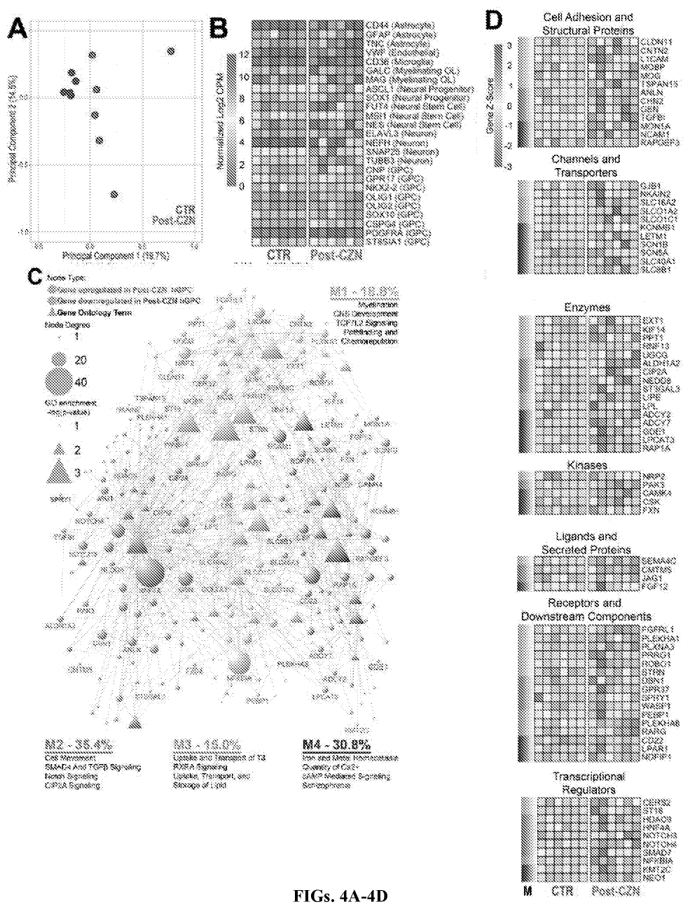

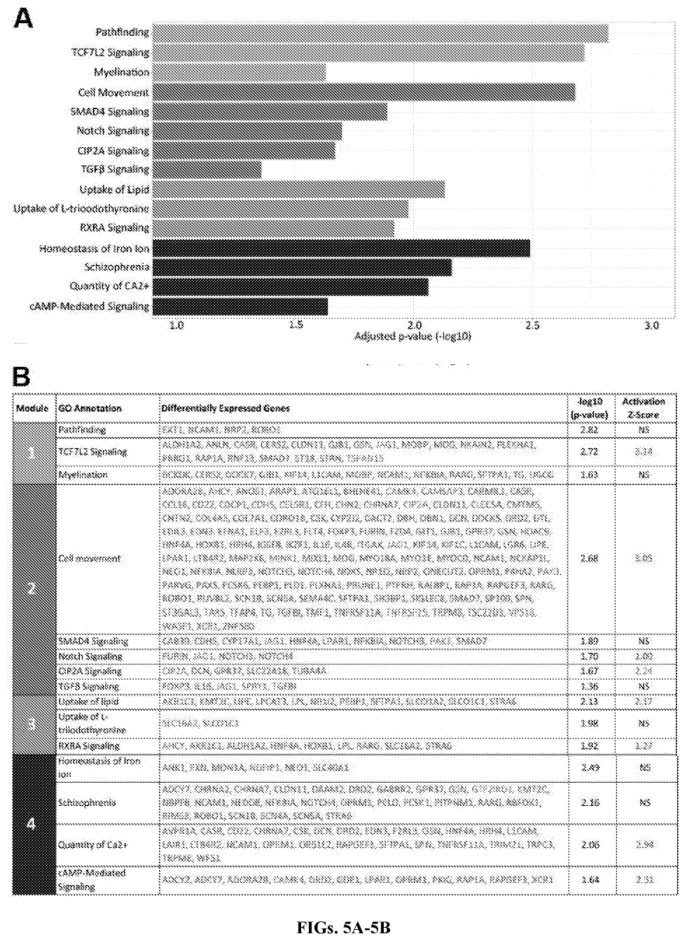

Principle component analysis of these normalized RNA-Seq samples revealed tight clustering of CTR samples, which as a group were readily distinguished from their post-CZN counterparts (FIG. 4A). Both the post-CZN and CTR hGPCs were enriched for genes associated with early oligodendroglial lineage, including CNP, GPR17, NKX2-2, OLIG1, OLIG2, SOX10, CSPG4, ST8SIA1, as well as the selection marker PDGFRA/CD140a, the latter validating the selectivity and efficacy of the sort (FIG. 4B). In contrast, the hGPC isolates exhibited low to undetectable expression of a number of neural stem cell, neural progenitor, endothelial, microglial, neuronal, and astrocytic markers. Yet while both groups presented with transcriptional signatures consistent with hGPC phenotype, a total of 914 transcripts were found to be differentially expressed between hGPCs following recovery from cuprizone treatment and their control counterparts (adjusted p<0.05). Of these, 777 genes were upregulated in cuprizone-treated GPCs, while 137 genes were down-regulated. Functional analysis of this gene set demonstrated that the cuprizone-treated hGPCs differentially expressed gene ontologies reflecting cell proliferation, pathfinding and cell movement, and the initiation of myelination itself (FIG. 5; and Table 5).

TABLE 5

| | | | −log10 | CZN Activation | |
|---|---|---|---|---|---|
| GO Annotation | Up regulated In CZN | Downregulated In CZN | (pvalue) | Z-Score | Module |
| Charcot Marie | GJB1, PDK3 | NA | 3.12 | 0.00 | I |
| Tooth disease X | | | | | |
| linked | | | | | |
| Pathfinding | EXT1, NCAM1, NRP2, ROBO1 | NA | 2.82 | 0.00 | 1 |
| TCF7L2 Signaling | ALDHIA2 ANLN, CASR, CERS2. CLDN11, GJB1, | JAG1, TSPAN15 | 2.72 | 3.14 | 1 |
| | GSN, MOBP, MOG, NKAIN2, PLEKHA1 PRRG1 | | | | |
| | RAP1A, RNF13, SMAD7, ST18, STRN | | | | |
| Fasciculation of | CNTN2, FEZ1, L1CAM, NCAM1, NRP2 | NA | 2.71 | 0.00 | 1 |
| axons | | | | | |
| Chemorepulsion | NRP2, PLXNA3, ROBO1, SCN1B | NA | 1.91 | 0.00 | 1 |
| Abnormal | GKB1, L1CAM, LPAR1 | NA | 1.84 | 0.00 | I |
| morphology of | | | | | |
| Schwann | | | | | |
| Formation of brain | AK8, ALDH1A2, DRD2, EFNA1, EXT1, FGFRLI, | RTN4RL2 | 1.72 | 0.00 | 1 |
| | FLT4, HCN1, HOXB1, IKZF1, KIF14, L1CAM, | | | | |
| | MECOM, NCAM1, NEO1, NME5, NOTCH3, NRP2, | | | | |
| | OR51E2, PAX5, PITPNM1, PLXNA3, PPTI, | | | | |
| | RAPGEF3, ROBO1, SEMA4C, SMG9, TG, ZIC4 | | | | |
| Complexity of | NRP2, PLDI, UGCG | NA | 1.72 | 0.00 | 1 |
| dendrites | | | | | |
| Myelination | BCKDK, CERS2, DOCK7, GJB1, KIF14, L1CAM, | NA | 1.63 | 0.00 | 1 |
| | MOBP, NCAM1, NFKBIA, RARG, SFTPA1, TG, | | | | |
| | UGCG | | | | |
| Guidance of axons | ANOS1, CNTN2, EXT1, FEZ1, H52STI, L1CAM, | NA | 1.62 | 0.00 | 1 |
| | LGI1, NRP2, PLXNA3, ROBO1, SCN1B | | | | |
| Development of | AK8, ALDH1A2, CELSR1, DRD2, EFNA1, EXTI, | RTN4RL2 | 1.58 | 2.61 | 1 |
| central nervous | FGFRL1, FLT4, GJB1, HCN1, HOXB1, HPCAL4, | | | | |
| system | IKZF1, KIF14, L1CAM, MECOM, MOG, NCAM1, | | | | |
| | NEO1, NME5, NOTCH3, NRP2, OR5IE2, PAX5, | | | | |
| | PDGFC, PTPNMI, PLXNA3, PPT1, PSPN, | | | | |
| | RAPGEF3, ROBO1, SEMA4C, SMG9, TG, ZIC4 | | | | |
| Cell movement | ADORA2B, ANOS1, ARAP1, ATG16L1, BHLHE41, | AHCY, CAMK4, | 2.68 | 3.05 | 2 |
| | CARMIL1, CASR, CCL16, CD22, CDCP1, CDH5, | CAMSAP3, CIP2A, | | | |
| | CELSR1, CFH, CHN2, CHRNA7, CLDN11, | IAG1, MINK1, PAK3, | | | |
| | CLEC5A, CMTM5, CNTN2, COL4A3, COL7A1, | TAR5, TNFRSF25, | | | |
| | CORO1B, CSK, CYP2J12, DACT2, DBH, DBN1, | VPS18 | | | |
| | DCN, DOCK5, DRD2, DTL, EDIL3, EDN3, EFNA1, | | | | |
| | ELF3, F2RL3, FLT4, FOXP3, FURIN, FZD4, GIT1, | | | | |
| | GJB1, GPR37, GSN, HDAC9, HNF4A, HOXB1, | | | | |
| | HRH4, IGSF8, IKZF1, IL16, IL4R, ITGAX, KIF14, | | | | |
| | KIF1C, L1CAM, LGR6, LIPE, LPAR1, LTB4R2, | | | | |
| | MAP2K6, MIXL1, MOG, MYO18A, MYO1E. | | | | |
| | MYOCD, NCAM1, NCKAP1L, NEO1, NFKBIA, | | | | |
| | NLRP3, NOTCH3, NOTCH4, NOX5, NR1I2, NRP2, | | | | |
| | ONECUT2, OPRM1, P4HA2, PARVG, PAX5, | | | | |
| | PCSK6, PEBPI, PLD1, PLXNA3, PRUNE1, PTPRH, | | | | |
| | RALBP1, RAP1A, RAPGEF3, RARG, ROBO1, | | | | |
| | RUVBL.2, SCNFB, SCN5A, SEMA4C, SFTPA1, | | | | |
| | SH3BP1, SIGLEC8, SMAD7, SP100, SPN, | | | | |
| | ST3GAL3, TFAP4, TG, TGFB1, TMF1, | | | | |
| | TNFRSF11A, TRPM8, TSC22D3, WASF1, XCR1, | | | | |
| | ZNF580 | | | | |
| Quantity of focal | CDH5, CSK, GIT1, MYO18A, PLD1 | NA | 2.08 | −1.39 | 2 |
| adhesions | | | | | |
| SMAD4 Signaling | CAB39, CDH5, CYP17A1, HNF4A, LPAR1, | IAG1, PAK3 | 1.89 | 0.00 | 2 |
| | NFKBIA, NOTCH3, SMAD7 | | | | |
| Notch Signaling | FURIN, NOTCH3, NOTCH4 | IAG1 - | 1.70 | 1.00 | 2 |
| CIP2A Signaling | DCN, GPR37, SLC22A16, TUBA4A | CIP2A | 1.67 | −2.24 | 2 |
| Polymerization of | CARMIL1, DBN1, FMN1, GSN, NCKAP1L, WASF1 | PAK3 | 1.67 | 1.97 | 2 |
| actin filaments | | | | | |
| Movement of | DBN1,KIFIC, MYO1E | NA | 1.67 | 0.00 | 2 |
| filaments | | | | | |
| Quantity of | GIT1, NRP2, WASF1 | PAK3 | 1.59 | 1.13 | 2 |
| dendritic spines | | | | | |
| GAP Junction | ADCY2, ADCY7, DBNI, DRD2, GJA5, GJB1, | NA | 1.38 | 0.00 | 2 |
| Signaling | LPAR1, PLCD3, TUBA4A, TUBB1 | | | | |
| TGFbeta signaling | FOXP3, IL16, TGFB1 | JAG1, SPRYI | 1.36 | 0.00 | 2 |
| Transport of | SLC16A2, SLCO1A2, SLCO1C1 | NA | 2.67 | 0.00 | 3 |
| thyroid hormone | | | | | |
| Storage of lipid | DGAT2, LIPE, LPL, NFKBIA, TMEM135, UGCG | SOAT1 | 2.25 | 1.25 | 3 |
| Accumulation of | CASR, LIPE | NA | 2.15 | 0.00 | 3 |
| diacylglycerol | | | | | |
| Uptake of lipid | AKR1C1, KMT2C, LIPE, LPCAT3, LPL, NR1I2, | NA | 2.13 | 2.17 | 3 |
| | PEBPI, SFTPA1, SLCO1A2, SLCO1C1, STRA6 | | | | |
| Uptake of L- | SLC16A2, SLCO1C1 | NA | 1.98 | 0.00 | 3 |
| triiodothyronine | | | | | |
| Uptake of | PEBP1, SFTPA1 | NA | 1.98 | 0.00 | 3 |
| phospholipid | | | | | |
| RXRA Signaling | AKR1C1, ALDH1A2, HNF4A, HOXB1, LPL, | AHCY | 1.92 | 1.27 | 3 |
| | RARG, SLC16A2, STRA6 | | | | |
| Transport of lipid | ACAT1, ACBD3, AKR1C1, GSN, LIPE, LPL, | APOC2, PLEKHAB, | 1.86 | 1.11 | 3 |
| | NFKBIA, PITPNMI, PLD1, SLC13A5, SLCO1A2, | SOAT1 | | | |
| | SLCO1C1, STRA6 | | | | |
| Transport of L- | ACAT1, ACBD3, AKR1C1, GSN, LIPE, LPL, | APOC2, PLEKHAB, | 1.86 | 1.11 | 3 |
| Transport of lipid | NFKBIA, PITPNMI, PLD1, SLC13A5, SLCO1A2, | SOAT1 | | | |
| | SLCO1C1, STRA6 | | | | |
| Transport of L- | SLC16A2, SLCO1C1 | NA | 1.84 | 0.00 | 3 |
| triiodothyronine | | | | | |
| Catabolism of | LIPE, LPL | NA | 1.62 | 0.00 | 3 |
| triacylglycerol | | | | | |
| UDP-N-acetyl D- | GNPDA1, GNPNAT1 | NA | 1.51 | 0.00 | 3 |
| galactosamine | | | | | |
| Export of metal | CD22, LETM1, SCN5A, TG | SLC8131 | 2.91 | 1.22 | 4 |
| ion | | | | | |
| Homeostasis of | ANK1, FXN, MON1A, NEO1, SLC4OA1 | NDFIP1 | 2.49 | 0 001 | 4 |
| Iron Ion | | | | | |
| Homeostasis of | ANK1, CASR, CHRNA7, EDN3, FXN, HNF4A, | NDFIP1 | 2.48 | 0.00 | 4 |
| metal ion | MON1A, NEO1, SCN5A, SLC40A1, TFAP2B, | | | | |
| | TMPRSS3, TRPM8, WFSI | | | | |
| Schizophrenia | ADCY7, CHRNA2, CHRNA7, CLDN11, DAAM2, | GTF2IRD1 | 2.16 | 0.00 | 4 |
| | DRD2, GABRR2, GPR37, GSN, KMT2C, NBPFB, | | | | |
| | NCAM1, NEDD8, NFKBIA, NOTCH4, OPRM1, | | | | |
| | PCLO, PCSK1, PITPNM1, RARG, RBFOX1, RIMS3, | | | | |
| | ROBO1, SCN1B, SCN4A, SCN5A, STRA6 | | | | |
| Quantity of Ca2+ | AVPR1A, CASR, CD22, CHRNA7, CSK, DCN, | TRIM21 | 2.06 | 2 94 | 4 |
| | DRD2, EDN3, F2RL3, GSN, HNF4A, HRH4, | | | | |
| | L1CAM, LAIR1, LTB4R2, NCAM1, OPRM1, | | | | |
| | OR51E2, RAPGEF3, SFTPA1, SPN, TNFRSFIIA, | | | | |
| | TRPC3, TRPM8, WFS1 | | | | |
| Ataxia | CHRNA7, CRX, DRD2, FXN, FZD4, KIF14, KIFIC, | CAMK4 | 2.00 | −2 16 | 4 |
| | NCAM1, PITPN1, SCN1B, SCN4A, SCN5A, | | | | |
| | SPRN, TDP1, TRPC3, ZIC4 | | | | |
| Quantity of metal | AVPRIA, CASR, CD22, CHRNA7, CSK, DCN, | NDFIP1, SL8B1, | 1.90 | 2 74 | 4 |
| Signaling | LPAR1, OPRM1, PK1G, RAP1A, RAPGEF3, XCR1 | TRIM21 | | | |
| Iron overload of | NEO1, SLC40A1 | NA | 1.62 | 0.00 | |
| liver | | | | | |
| Transport of metal | ANK1, CASR, CD22, CHRNA7, DRD2, FGF12, | NDFIP1, SLC8B1 | 1.57 | 2.43 | 4 |
| | FXN, GJA5, G1JB1, KCNMB1, LETM1, MON1A, | | | | |
| | NEO1, SCNFB, SCN4A, SCN5A, SLC40A1, TG, | | | | |
| | TRPC3, TRPM8 | | | | |
| G-Protein Coupled | ADCY2, ADCY7, ADORA2B, AVPRIA, DRD2, | CAMK4 | 1.39 | 0.00 | 4 |
| Receptor Signaling | GDE1, LPAR1, NFKBIA, OPRM1, RAP1A, | | | | |
| | RAPGEF3, XCR1 | | | | |

### Example 5—Network Analysis Revealed that Cuprizone-Exposed hGPCs were Primed to Oligoneogenesis

To aid in interpreting these data, a cuprizone-exposed hGPC expression network was constructed based upon both significantly enriched gene ontologies and differentially-expressed individual gene components thereof. The network included 43 significantly enriched and relevant functional terms, in addition to their contributing differentially expressed genes (network in FIG. 4C; functionally-segregated heat-maps in FIG. 4D; complete gene ontology network table in Table 5). Community detection via modularity analysis was then carried out to aggregate closely related functions and genes (Bastian et al., “Gephi: An Open Source Software for Exploring and Manipulating Networks,” Proceedings of the Third International ICWSM Conference 361-362 (2009); Blondel et al., “Fast Unfolding of Communities in Large Networks,” Journal of Statistical Mechanics: Theory and Experiment Issue 10, pp. 10008, 12 pp. (2008), which are hereby incorporated by reference in their entirety). This analysis yielded four distinct modules (M1-M4), which individually identified distinct processes associated with the initiation of remyelination by cuprizone demyelination-mobilized hGPCs.

M1 revealed that the hGPCs recovering from cuprizone demyelination markedly upregulated their expression of myelinogenesis-associated genes, including MOG, MOBP, and CLDN11 (Goldman, S.A., “How to Make an Oligodendrocyte,” Development 142:3983-3995 (2015), which is hereby incorporated by reference in its entirety) (FIG. 4C). Furthermore, several genes previously noted to be induced during oligodendrocyte differentiation and remyelination were also upregulated; these included ST18 (Najm et al., “Transcription Factor-Mediated Reprogramming of Fibroblasts to Expandable, Myelinogenic Oligodendrocyte Progenitor Cells,” Nature Biotechnology 31:426-433 (2013), which is hereby incorporated by reference in its entirety), PLEKHA1 (Chen et al., “TAPP1 Inhibits the Differentiation of Oligodendrocyte Precursor Cells Via Suppressing the Mek/Erk Pathway,” Neuroscience Bulletin 31:517-526 (2015), which is hereby incorporated by reference in its entirety), and CMTM5 (Doyle et al., “Application of a Translational Profiling Approach for the Comparative Analysis of CNS Cell Types,” Cell 135:749-762 (2008), which is hereby incorporated by reference in its entirety), along with those shown to be necessary for appropriate maturation of oligodendrocytes: CERS2 (Imgrund et al., “Adult Ceramide Synthase 2 (CERS2)-Deficient Mice Exhibit Myelin Sheath Defects, Cerebellar Degeneration, and Hepatocarcinomas,” The Journal of Biological Chemistry 284:33549-33560 (2009), which is hereby incorporated by reference in its entirety), LPAR1 (Garcia-Diaz et al., “Loss of Lysophosphatidic Acid Receptor LPA1 Alters Oligodendrocyte Differentiation and Myelination in the Mouse Cerebral Cortex,” Brain Structure & Function 220:3701-3720 (2015), which is hereby incorporated by reference in its entirety), GSN (Zuchero et al., “CNS Myelin Wrapping is Driven by Actin Disassembly,” Developmental Cell 34:152-167 (2015), which is hereby incorporated by reference in its entirety), KIF14 (Fujikura et al., “Kif14 Mutation Causes Severe Brain Malformation and Hypomyelination,” PLoS ONE 8:e53490 (2013), which is hereby incorporated by reference in its entirety), CNTN2 (Zoupi et al., “The Function of Contactin-2/TAG-1 in Oligodendrocytes in Health and Demyelinating Pathology,” Glia 66:576-591 (2018), which is hereby incorporated by reference in its entirety), ST3GAL3 (Yoo et al., “Sialylation Regulates Brain Structure and Function,” FASEB Journal: Official Publication of the Federation of American Societies for Experimental Biology 29:3040-3053 (2015), which is hereby incorporated by reference in its entirety), and WASF1/WAVE1 (Kim et al., “WAVE1 is Required for Oligodendrocyte Morphogenesis and Normal CNS Myelination,” The Journal of Neuroscience: The Official Journal of the Society for Neuroscience 26:5849-5859 (2006), which is hereby incorporated by reference in its entirety). Interestingly, M1 further revealed that TCF7L2 signaling, a major driver of myelination (Hammond et al., “The Wnt Effector Transcription Factor 7-like 2 Positively Regulates Oligodendrocyte Differentiation in a Manner Independent of Wnt/Beta-Catenin Signaling,” The Journal of Neuroscience: the Official Journal of the Society for Neuroscience 35:5007-5022 (2015); Zhao et al., “Dual Regulatory Switch Through Interactions of Tcf712/Tcf4 with Stage-Specific Partners Propels Oligodendroglial Maturation,” Nature Communications 7:10883 (2016), which are hereby incorporated by reference in their entirety), is also strongly upregulated in remyelinating hGPCs. Genes contributing to the migration and pathfinding of GPCs were also up-regulated following cuprizone exposure; these included ROBO1 (Liu et al., “Slit2 Regulates the Dispersal of Oligodendrocyte Precursor Cells Via Fyn/RhoA Signaling,” The Journal of Biological Chemistry 287:17503-17516 (2012), which is hereby incorporated by reference in its entirety) and the class 3 semaphorin co-receptors NRP2 (Boyd et al. “Insufficient OPC Migration into Demyelinated Lesions is a Cause of Poor Remyelination in MS and Mouse Models,” Acta Neuropathologica 125:841-859 (2013), which is hereby incorporated by reference in its entirety) and PLXNA3 (Xiang et al., “Plexin A3 is Involved in Semaphorin 3F-Mediated Oligodendrocyte Precursor Cell Migration,” Neuroscience Letters 530:127-132 (2012), which is hereby incorporated by reference in its entirety).

Within M2, a number of transcription factors and associated signal effectors vital to oligodendrocyte movement and differentiation were noted to be significantly enriched in the post cuprizone hGPCs. These included SMAD4 (Choe et al., “Migration of Oligodendrocyte Progenitor Cells is Controlled by Transforming Growth Factor Beta Family Proteins During Corticogenesis,” The Journal of Neuroscience: the Official Journal of the Society for Neuroscience 34:14973-14983 (2014), which is hereby incorporated by reference in its entirety), TGFβ (McKinnon et al., “A Role for TGF-Beta in Oligodendrocyte Differentiation,” The Journal of Cell Biology 121:1397-1407 (1993), which is hereby incorporated by reference in its entirety), and NOTCH (Park et al., “Delta-Notch Signaling Regulates Oligodendrocyte Specification,” Development 130:3747-3755 (2003), which is hereby incorporated by reference in its entirety), as well as the pro-myelinogenic genes SMAD7 (Weng et al., “Dual-Mode Modulation of Smad Signaling by Smad-Interacting Protein Sipl is Required for Myelination in the Central Nervous System,” Neuron 73:713-728 (2012), which is hereby incorporated by reference in its entirety), PAK3 (Maglorius et al., “The Intellectual Disability Protein PAK3 Regulates Oligodendrocyte Precursor Cell Differentiation,” Neurobiology of Disease 98:137-148 (2017), which is hereby incorporated by reference in its entirety), and NOTCH3 (Zaucker et al., “Notch3 is Essential for Oligodendrocyte Development and Vascular Integrity in Zebrafish,” Disease Models & Mechanisms 6:1246-1259 (2013), which is hereby incorporated by reference in its entirety). In contrast, the Notch pathway inhibitor of differentiation JAG1 was sharply repressed (John et al., “Multiple Sclerosis: Re-Expression of a Developmental Pathway that Restricts Oligodendrocyte Maturation,” Nat Med 8:1115-1121 (2002), which is hereby incorporated by reference in its entirety), suggesting the incipient differentiation of these cells. Also localizing to this module and upregulated in remyelinating GPCs was GPR37, the expression of which attends and is necessary for oligodendrocyte differentiation (Smith et al., “Mice Lacking Gpr37 Exhibit Decreased Expression of the Myelin-Associated Glycoprotein MAG and Increased Susceptibility to Demyelination,” Neuroscience 358:49-57 (2017); Yang et al., “G Protein-Coupled Receptor 37 is a Negative Regulator of Oligodendrocyte Differentiation and Myelination,” Nature Communications 7:10884 (2016), which are hereby incorporated by reference in their entirety). Interestingly, CIP2A, a transcriptional repressor of GPR37, was profoundly down-regulated in cuprizone-mobilized hGPCs, again suggesting that cuprizone-demyelination triggers the active disinhibition of oligodendrocytic differentiation by previously quiescent parenchymal hGPCs (Yang et al., “G Protein-Coupled Receptor 37 is a Negative Regulator of Oligodendrocyte Differentiation and Myelination,” Nature Communications 7:10884 (2016), which is hereby incorporated by reference in its entirety).

M3 consisted of several functional categories strongly involved in myelination. These included transcripts involved in the transport and uptake of thyroid hormone and L-triiodothyronine (Almazan et al., “Triiodothyronine Stimulation of Oligodendroglial Differentiation and Myelination. A Developmental Study,” Developmental Neuroscience 7:45-54 (1985); Bhat et al., “Investigations on Myelination in Vitro. Regulation by Thyroid Hormone in Cultures of Dissociated Brain Cells from Embryonic Mice,” J Biol Chem 254:9342-9344 (1979), which are hereby incorporated by reference in their entirety). M3 also included genes associated with retinoid-signaling, particularly RXRA and the retinoid receptor complex partner RARG, the up-regulation of which was observed in hGPCs after cuprizone treatment (Huang et al., “Retinoid X Receptor Gamma Signaling Accelerates CNS Remyelination,” Nature Publishing Group 14:45-53 (2011); Tomaru et al., “Identification of an Inter-Transcription Factor Regulatory Network in Human Hepatoma Cells by Matrix RNAi,” Nucleic Acids Research 37:1049-1060 (2009), which are hereby incorporated by reference in their entirety). This is of particular significance as this signaling family has previously been tied not only to developmental myelination (De La Fuente et al., “Vitamin D Receptor-Retinoid X Receptor Heterodimer Signaling Regulates Oligodendrocyte Progenitor Cell Differentiation,” J Cell Biol 211:975-985 (2015), which is hereby incorporated by reference in its entirety), but also to remyelination as well (Huang et al., “Retinoid X Receptor Gamma Signaling Accelerates CNS Remyelination,” Nature Publishing Group 14:45-53 (2011), which is hereby incorporated by reference in its entirety). M3 also included genes associated with cholesterol and lipid uptake, processes critical to myelination (Saher et al., “High Cholesterol Level is Essential for Myelin Membrane Growth,” Nature Neuroscience 8:468-475 (2005), which is hereby incorporated by reference in its entirety).

The fourth module included genes associated with the transport and homeostatic regulation of iron and other multivalent cations, which were upregulated following cuprizone demyelination. Iron in particular has been reported to be important in the regulation of oligodendrocytic differentiation and myelination (Connor et al., “Development of Transferrin-Positive Oligodendrocytes in the Rat Central Nervous System,” Journal of Neuroscience Research 17:51-59 (1987); Morath et al., “Iron Modulates the Differentiation of a Distinct Population of Glial Precursor Cells into Oligodendrocytes,” Developmental Biology 237:232-243 (2001), which are hereby incorporated by reference in their entirety). In this regard, upregulation of MON1A and FXN, iron metabolism-associated genes strongly dysregulated in MS lesions (Hametner et al., “Iron and Neurodegeneration in the Multiple Sclerosis Brain,” Annals of Neurology 74:848-861 (2013), which is hereby incorporated by reference in its entirety), was observed in cuprizone-mobilized hGPCs. Similarly, it was noted that genes encoding calcium regulatory proteins associated with myelin maturation, which included CASR, GSN, and TRPC3 (Cheli et al., “Voltage-Gated Ca2+Entry Promotes Oligodendrocyte Progenitor Cell Maturation and Myelination in Vitro,” Experimental Neurology 265:69-83 (2015); Krasnow et al., “Regulation of Developing Myelin Sheath Elongation by Oligodendrocyte Calcium Transients in Vivo,” Nature Neuroscience 21:24-28 (2018), which are hereby incorporated by reference in their entirety), were also increased in hGPCs after cuprizone demyelination, as were transcripts involved in cAMP signaling, another modulator of oligodendrocyte differentiation, in part via crosstalk with GPR37 and GPR17 (Simon et al., “The Orphan G Protein-Coupled Receptor GPR17 Negatively Regulates Oligodendrocyte Differentiation Via Gai/o and its Downstream Effector Molecules,” Journal of Biological Chemistry 291:705-718 (2016); Yang et al., “G Protein-Coupled Receptor 37 is a Negative Regulator of Oligodendrocyte Differentiation and Myelination,” Nature Communications 7:10884 (2016), which are hereby incorporated by reference in their entirety).

Overall, the pattern of differential gene expression by cuprizone-exposed hGPCs reflected in FIG. 4 appears to define an expression network typifying that of early progenitor-derived remyelination. As such, these data indicate that when mobilized in response to antecedent cuprizone demyelination, human GPCs activated a coherent set of transcriptional programs that served to direct both oligodendrocytic differentiation and myelinogenesis.

### Discussion of Examples

The congenitally hypomyelinated shiverer mouse (MBP^{shi/shi}) is a naturally-occurring mutant that lacks myelin basic protein (MBP), and as such cannot make compact myelin. It has been found that the intracerebral injection of hGPCs into neonatal shiverer mice results in the widespread dispersal of the human donor cells, followed by their oligodendrocytic differentiation and myelinogenesis (Windrem et al., “Fetal and Adult Human Oligodendrocyte Progenitor Cell Isolates Myelinate the Congenitally Dysmyelinated Brain,” Nat Med 10:93-97 (2004); Windrem et al., “Neonatal Chimerization with Human Glial Progenitor Cells can both Remyelinate and Rescue the Otherwise Lethally Hypomyelinated Shiverer Mouse,” Cell Stem Cell 2:553-565 (2008), which are hereby incorporated by reference in their entirety). This ultimately leads to the complete or near-complete myelination of the recipient's brain, brainstem and spinal cord, attended by the clinical rescue of a large proportion of transplanted neonates. Yet notwithstanding the robust developmental myelination of the host CNS by neonatally-delivered hGPCs, in order for hGPC delivery to be a viable regenerative strategy for treating adult demyelination—especially as occurs in multicentric and diffuse myelin loss—then the donor cells must be capable of migrating within and myelinating adult brain parenchyma.

In adults, oligodendrocytic loss contributes to diseases as diverse as hypertensive and diabetic white matter loss, traumatic spinal cord and brain injury, multiple sclerosis (MS) and its variants, and even the age-related white matter loss of the subcortical dementias. All of these conditions are potential targets of glial progenitor cell replacement therapy, recognizing that the adult disease environment may limit this approach in a disease-specific fashion (Goldman, S.A., “Progenitor Cell-Based Treatment of Glial Disease,” Prog Brain Res 231:165-189 (2017); Goldman et al., “Glial Progenitor Cell-Based Treatment and Modeling of Neurological Disease,” Science 338:491-495 (2012), which are hereby incorporated by reference in their entirety). For instance, the chronically ischemic brain tissue of diabetics with small vessel disease may require aggressive treatment of the underlying vascular insufficiency before any cell replacement strategy may be considered. Similarly, the inflammatory disease environments of multiple sclerosis as well as many of the leukodystrophies present their own challenges, which need to be overcome before cell-based remyelination can succeed (Franklin et al., “Remyelination in the CNS: from Biology to Therapy,” Nat Rev Neurosci 9:839-855 (2008); Franklin et al., “Regenerating CNS Myelin—from Mechanisms to Experimental Medicines,” Nat Rev Neurosci 18:753-769 (2017); Goldman, S.A., “Stem and Progenitor Cell-Based Therapy of the Central Nervous System: Hopes, Hype, and Wishful Thinking,” Cell Stem Cell 18:174-188 (2016); Ip et al., “Immune Cells Contribute to Myelin Degeneration and Axonopathic Changes in Mice Overexpressing Proteolipid Protein in Oligodendrocytes,” The Journal of Neuroscience 26:8206-8216 (2006), which are hereby incorporated by reference in their entirety); these include participation by mobilized GPCs in the inflammatory response, potentially complicating therapeutic efforts further (Falcao et al., “Disease-specific Oligodendrocyte Lineage Cells Arise in Multiple Sclerosis,” Nat Med 24:1837-1844 (2018), which is hereby incorporated by reference in its entirety). Nonetheless, current disease-modifying strategies for treating both vascular and autoimmune diseases have advanced to the point where stabilization of the disease environment can often be accomplished, such that transplant-based remyelination for the structural repair of demyelinated adult white matter may now be feasible.

Despite concerns as to the ability of glial progenitor cells to remyelinate axons in disease environments such as those associated with multiple sclerosis and the periventricular leukomalacia of cerebral palsy, a number of studies have pointed to the cell-intrinsic nature of oligodendrocytic differentiation block in these cases. These studies have suggested that the inability of parenchymal glial progenitors to produce myelinating oligodendrocytes in these conditions is a function of stable epigenetic blocks in the differentiation potential of these cells, imparted by the specific disease process or its antecedents. Newly introduced naïve hGPCs might thus be expected to exercise unfettered differentiation and myelination competence in host brains, and as such be able to remyelinate previously demyelinated axons. Indeed, several prior studies have indicated the ability of transplanted oligodendrocyte progenitors to remyelinate adult-demyelinated central axons (Duncan et al., “Extensive Remyelination of the CNS Leads to Functional Recovery,” Proc Natl Acad Sci USA 106:6832-6836 (2009); Mozafari et al., “Skin-Derived Neural Precursors Competitively Generate Functional Myelin in Adult Demyelinated Mice,” J Clin Invest 125:3642-3656 (2015), which are hereby incorporated by reference in their entirety). To define the competence of human GPCs to remyelinate axons when delivered to the demyelinated adult brain, two different antigenic phenotypes of GPCs were used, respectively defined as CD140a+ and A2B5+/PSA-NCAM−, each derived from fetal human brain tissue (Sim et al., “CD140a Identifies a Population of Highly Myelinogenic, Migration-Competent and Efficiently Engrafting Human Oligodendrocyte Progenitor Cells,” Nature Biotechnology 29:934-941 (2011); Windrem et al., “Neonatal Chimerization With Human Glial Progenitor Cells Can Both Remyelinate and Rescue the Otherwise Lethally Hypomyelinated Shiverer Mouse,” Cell Stem Cell 2:553-565 (2008), which are hereby incorporated by reference in their entirety). These antigenic phenotypes are largely but not completely homologous; the CD140a phenotype is the major fraction of, and largely subsumed within, the A2B5+/PSA-NCAM-pool (Sim et al., “CD140a Identifies a Population of Highly Myelinogenic, Migration-Competent and Efficiently Engrafting Human Oligodendrocyte Progenitor Cells,” Nature Biotechnology 29:934-941 (2011), which is hereby incorporated by reference in its entirety). The dispersal and myelination competence of these cell types was assessed in two distinct adult models of myelin deficiency, the congenitally hypomyelinated shiverer mouse as well as the normally myelinated adult mouse, and the cuprizone-treated demyelinated adult mouse.

In each of these model systems, the transplanted hGPCs effectively restored myelin to the host brain. Donor-derived myelination was robust when cells were delivered to the adult shiverer brain, just as previously reported after hGPC transplants in neonatal shiverer mice. The design of this experiment was intended to mimic what might be encountered in the postnatal treatment of a hypomyelinating leukodystrophy, and the effective progenitor cell dispersal and myelination that was observed augurs well for the potential of this approach in the treatment of children with congenital leukodystrophies. Similarly, donor hGPC-derived oligodendrocyte differentiation and axonal remyelination proved robust in response to cuprizone demyelination, whether by hGPCs already resident within the adult-demyelinated brains, or by those transplanted during and after demyelination. Together, this latter set of experiments in particular provided an important proof-of principle, showing that hGPCs could remyelinate axons that had already been myelinated in the past, and which were then demyelinated in the setting of oligodendrocyte loss; precisely such a scenario might be anticipated in disorders such as progressive multiple sclerosis, in which the remyelination of stably-denuded residual axons might be expected to confer functional benefit.

Indeed, in each of these experimental paradigms it was found that the hGPCs, whether engrafted neonatally or transplanted into adults, effectively dispersed throughout the forebrains, even in normally myelinated mice, and differentiated as oligodendroglia and myelinated demyelinated lesions as these evolved or were encountered. These data suggest that transplanted fetal hGPCs are competent to disperse broadly and differentiate as myelinogenic cells in the adult brain, and—critically—that they are able to remyelinate previously myelinated axons that have experienced myelin loss.

Having established the potential of human GPCs as myelinogenic vectors, RNAseq of hGPCs extracted from the brains in which they had been resident during cuprizone exposure was then used to assess the transcriptional response of these cells to demyelination and initial remyelination. By this means, the demyelination-associated recruitment of resident hGPCs was correlated with their coincident transcriptional responses, so as to identify—in human cells, isolated directly from the in vivo environment—those genes and pathways whose targeting might permit the therapeutic modulation of both progenitor recruitment and differentiated fate.

It was found that mobilized human GPCs indeed expressed a transcriptional signature consistent with early remyelination, as has been noted with demyelination-mobilized murine progenitors as well (Moyon et al., “Demyelination Causes Adult CNS Progenitors to Revert to an Immature State and Express Immune Cues That Support Their Migration,” The Journal of Neuroscience: The Official Journal of the Society for Neuroscience 35, 4-20 (2015), which is hereby incorporated by reference in its entirety). Yet human and mouse glial progenitors are quite different phenotypes, and are distinct in their transcriptional signatures (Lovatt et al., “The Transcriptome and Metabolic Gene Signature of Protoplasmic Astrocytes in the Adult Murine Cortex,” The Journal of Neuroscience: The Official Journal of the Society for Neuroscience 27:12255-12266 (2007); Sim et al., “Complementary Patterns of Gene Expression by Human Oligodendrocyte Progenitors and Their Environment Predict Determinants of Progenitor Maintenance and Differentiation,” Ann Neurol 59:763-779 (2006); Sim et al., “Fate Determination of Adult Human Glial Progenitor Cells,” Neuron Glia Biol 5:45-55 (2009); Zhang et al., “Purification and Characterization of Progenitor and Mature Human Astrocytes Reveals Transcriptional and Functional Differences with Mouse,” Neuron 89:37-53 (2016), which are hereby incorporated by reference in their entirety), lineage restriction (Nunes et al., “Identification and Isolation of Multipotential Neural Progenitor Cells from the Subcortical White Matter of the Adult Human Brain,” Nat Med 9:439-447 (2003), which is hereby incorporated by reference in its entirety), and daughter cell morphologies (Oberheim et al., “Uniquely Hominid Features of Adult Human Astrocytes,” The Journal of Neuroscience: The Official Journal of the Society for Neuroscience 29:3276-3287 (2009), which is hereby incorporated by reference in its entirety). Accordingly, a number of activated pathways were noted in demyelination-triggered hGPCs not previously noted in models of murine demyelination. These included a number of upstream directors of oligodendroglial fate choice and differentiation, that included TCF7L2, TGFß/SMAD4, and NOTCH driven pathways. The activation of each of these pathways was linked to the demyelination associated disinhibition of differentiation by these parenchymal hGPCs, the oligodendrocytic maturation of which then enabled the compensatory remyelination of denuded axons. In addition, besides the activation of differentiation-associated pathways in demyelination-stimulated hGPCs, the data also suggested the critical importance during early remyelination of pathways enabling iron transport and metabolism, as well as those facilitating cholesterol and lipid uptake, pathways that are critically important to oligodendrocytic myelinogenesis. Interestingly, the identification of these pathways as differentially upregulated during remyelination suggests that the efficiencies of both myelinogenesis and myelin maturation might be further potentiated via metabolic optimization, and potentially even by dietary modulation referable to these pathways. Together, these studies establish an operational rationale for assessing the ability of hGPCs to remyelinate demyelinated lesions of the adult human brain, while providing a promising set of molecular targets for the modulation of this process in human cells.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. A method of treating a human subject having a condition mediated by a deficiency in myelin, said method comprising:
selecting a human subject having a condition mediated by a deficiency in myelin and
administering to the selected subject one or more modulators of a cell signaling pathway selected from the group consisting of Notch signaling, cAMP mediated signaling, CIP2A signaling, RXRA signaling, TCF7L2 signaling, and combinations thereof under conditions effective to treat the condition.

2. A method of increasing oligodendrocyte production from human glial progenitor cells, said method comprising:
providing a population of human glial progenitor cells and
administering in vitro to the provided population of human glial progenitor cells, one or more modulators of a cell signaling pathway selected from the group consisting of Notch signaling, cAMP mediated signaling, CIP2A signaling, RXRA signaling, TCF7L2 signaling, and combinations thereof under conditions effective to increase oligodendrocyte production compared to oligodendrocyte production absent said administering.

3.-6. (canceled)

7. The method of claim 1, wherein the one or more modulators stimulate TCF7L2 signaling.

8.-19. (canceled)

20. The method of claim 7, wherein, as a result of said administering,
one or more genes selected from the group consisting of ACAA1, ADCY9, ALOX5, CD24, CPD, CYP51A1, DHCR24, EPAS1, ERBB3, GSN, GNAI1, HES1, IDH1, JAG1, MAN1A1, ME1, NOTCH1, NOTCH3, NPC1, PAPSS1, PLAT, SRD5A1, CAMK2N1, CAP1, CCND2, DOCKS, FGFR2, MAP7, PELI1, PPP1R16B, PRKAR2B, RAP1GAP, RPS6KA1, ACSL1, ADORA2B, ADRB2, APOD, ASPA, CCP110, ENPP2, FTH1, GNAS, HSPA2, IPO13, IRS2, MOBP, PLP1, PRKAR1A, PTGER4, ALDH1A1, CCND1, COLEC12, CRABP2, DDC, EGFR, FA2H, FABP4, GCLC, GPX1, HBEGF, LCN2, LPL, MAG7, MBP, MCAM, NPY, PDGFA, PMP22, QKI, S100A8, SLC6A8, WISP2, ALDH1A2, CDK19, CREB3L2, ENPP4, IGF1, KAT2B, NKX2-1, OLIG2, SH3GL3, SLC12A2, SLC27A2, SOX10, BIN1, CDK19, MAP4K4, MYO6, RAP2A, ST18, CCP110, DHRS7, JAM3, MCAM, PAPSS1, RNF13, SECISBP2L, ACSL3, GLUL, MMP7, NPC2, SPP1, UBE2G1, ANXA1, TCF7L2, TNS1, ADO, ELOVL1, KIF5B, LAMP1, STK39, TMEM123, AQP9, ASPH, DEGS1, HIPK2, KTN1, MAL, PLEKHB1, RNASE4, CSRP1, HMGCS2, NFASC, IRS1, NUDT4, EVI2A, MAG, MOG, RAB33A, TWF1, GCLM, SMAD7, PRRG1, LDLRAP1, EVI2A, RALGDS, CARHSP1, TBC1D5, ARAP2, ARHGEF10, CTNNAL1, PTPN11, GJB1, HMGCS2, RCBTB1, PICALM, POLR1D, MYC, ALOX5, SYPL1, SEMA4D, CHMP1B, SNAP23, SORBS3, and RAB31 are upregulated and/or one or more genes selected from the group consisting of ID1, ID4, PCK1, ID3, AQP3, CDKN1B, BMP4, KDM4B, FBP1, DUSP1, DUSP4, PKIG, PPP3CA, ABCC3, CCL20, TGFB2, DLK1, WEE1, APOA1, CXCL10, DLK1, ID2, and FH are downregulated.

21. The method of claim 7, wherein the one or more modulators are selected from the group consisting of Trichostatin A, BRD-K30523950, CI 976, Rolipram, AZD8055, BRD-K90999434, NSC 23766, Teniposide, BAS 00535043, BRD-K50177987, BRD-K76568384, 2541665-P2, BRD-K34495954, BRD-K59488055, DM161, BRD-K95212245, Idazoxan Hydrochloride, NCGC00182823-01, Thiazolopyrimidine, Wortmannin, 1503640, BRD-A19195498, BRD-A94413429, BRD-K21565985, BRD-K55612480, BRD-K61217870, BRD-K63326650, BRD-K71670746, BRD-K76587808, BRD-K76896292, BRD-K93480852, BRD-K98991361, INK-128, MLS-0327420.0002, MW-Ras9, NCGC00182845-01, Sertraline, Valproic Acid, BRD-K04853698, BRD-K74761218, Dasatinib, Geldanamycin, and JW-7-24-1.

22. The method of claim 21, wherein the one or more modulators are selected from the group consisting of Trichostatin A, BRD-K30523950, CI 976, Rolipram, BAS 00535043. BRD-K50177987, BRD-K76568384, 2541665-P2, BRD-K59488055, NCGC00182823-01, BRD-K21565985, BRD-K55612480, BRD-K61217870, BRD-K98991361, Sertraline, Dasatinib, Geldanamycin, JW-7-24-1, Wortmannin, Idazoxan Hydrochloride, MLS-0327420.0002, AZD8055, BRD-K90999434, NSC 23766, Teniposide, BRD-K34495954, BRD-K63326650, BRD-K76896292, and BRD-K74761218.

23. The method of claim 1, wherein said condition is selected from the group consisting of pediatric leukodystrophies, lysosomal storage diseases, congenital dysmyelination, cerebral palsy, inflammatory demyelination, post-infectious and post-vaccinial leukoencephalitis, radiation- or chemotherapy induced demyelination, and vascular demyelination.

24. The method of claim 1, wherein said subject has a condition requiring myelination.

25. The method of claim 24, wherein said condition is selected from the group consisting of Pelizaeus-Merzbacher Disease, Tay-Sach Disease, Sandhoff's gangliosidoses, Krabbe's disease, metachromatic leukodystrophy, mucopolysaccharidoses, Niemann-Pick A disease, adrenoleukodystrophy, Canavan's disease, Vanishing White Matter Disease, and Alexander Disease.

26. The method of claim 1, wherein said subject has a condition requiring remyelination.

27. The method of claim 26, wherein said condition is selected from the group consisting of multiple sclerosis, neuromyelitis optica, transverse myelitis, optic neuritis, subcortical stroke, diabetic leukoencephalopathy, hypertensive leukoencephalopathy, age-related white matter disease, spinal cord injury, radiation- or chemotherapy induced demyelination, post-infectious and post-vaccinial leukoencephalitis, periventricular leukomalacia, and cerebral palsy.

28. The method of claim 1 further comprising: administering to the selected subject a preparation of human glial progenitor cells.

29. The method of claim 28, wherein the preparation is administered to one or more sites of the brain, the brain stem, the spinal cord, or a combination thereof.

30. The method of claim 29, wherein the preparation is administered intraventricularly, intracallosally, or intraparenchymally.

31. The method of claim 28, wherein the glial progenitor cells are derived from fetal tissue.

32. The method of claim 28, wherein the glial progenitor cells are derived from embryonic stem cells.

33. The method of claim 28, wherein the glial progenitor cells are derived from induced pluripotent stem cells.

34. The method of claim 1, wherein said administering is carried out using intracerebral delivery, intrathecal delivery, intranasal delivery, or via direct infusion into brain ventricles.

35. The method of claim 2, wherein the one or more modulators stimulate TCF7L2 signaling.

36. The method of claim 35, wherein, as a result of said administering, one or more genes selected from the group consisting of ACAA1, ADCY9, ALOX5, CD24, CPD, CYP51A1, DHCR24, EPAS1, ERBB3, GSN, GNAI1, HES1, IDH1, JAG1, MAN1A1, ME1, NOTCH1, NOTCH3, NPC1, PAPSS1, PLAT, SRD5A1, CAMK2N1, CAP1, CCND2, DOCKS, FGFR2, MAP7, PELI1, PPP1R16B, PRKAR2B, RAP1GAP, RPS6KA1, ACSL1, ADORA2B, ADRB2, APOD, ASPA, CCP110, ENPP2, FTH1, GNAS, HSPA2, IPO13, IRS2, MOBP, PLP1, PRKAR1A, PTGER4, ALDH1A1, CCND1, COLEC12, CRABP2, DDC, EGFR, FA2H, FABP4, GCLC, GPX1, HBEGF, LCN2, LPL, MAG7, MBP, MCAM, NPY, PDGFA, PMP22, QKI, S100A8, SLC6A8, WISP2, ALDH1A2, CDK19, CREB3L2, ENPP4, IGF1, KAT2B, NKX2-1, OLIG2, SH3GL3, SLC12A2, SLC27A2, SOX10, BIN1, CDK19, MAP4K4, MYO6, RAP2A, ST18, CCP110, DHRS7, JAM3, MCAM, PAPSS1, RNF13, SECISBP2L, ACSL3, GLUL, MMP7, NPC2, SPP1, UBE2G1, ANXA1, TCF7L2, TNS1, ADO, ELOVL1, KIF5B, LAMP1, STK39, TMEM123, AQP9, ASPH, DEGS1, HIPK2, KTN1, MAL, PLEKHB1, RNASE4, CSRP1, HMGCS2, NFASC, IRS1, NUDT4, EVI2A, MAG, MOG, RAB33A, TWF1, GCLM, SMAD7, PRRG1, LDLRAP1, EVI2A, RALGDS, CARHSP1, TBC1D5, ARAP2, ARHGEF10, CTNNAL1, PTPN11, GJB1, HMGCS2, RCBTB1, PICALM, POLR1D, MYC, ALOX5, SYPL1, SEMA4D, CHMP1B, SNAP23, SORBS3, and RAB31 are upregulated and/or one or more genes selected from the group consisting of ID1, ID4, PCK1, ID3, AQP3, CDKN1B, BMP4, KDM4B, FBP1, DUSP1, DUSP4, PKIG, PPP3CA, ABCC3, CCL20, TGFB2, DLK1, WEE1, APOA1, CXCL10, DLK1, ID2, and FH are downregulated.

37. The method of claim 35, wherein the one or more modulators are selected from the group consisting of Trichostatin A, BRD-K30523950, CI 976, Rolipram, AZD8055, BRD-K90999434, NSC 23766, Teniposide, BAS 00535043, BRD-K50177987, BRD-K76568384, 2541665-P2, BRD-K34495954, BRD-K59488055, DM161, BRD-K95212245, Idazoxan Hydrochloride, NCGC00182823-01, Thiazolopyrimidine, Wortmannin, 1503640, BRD-A19195498, BRD-A94413429, BRD-K21565985, BRD-K55612480, BRD-K61217870, BRD-K63326650, BRD-K71670746, BRD-K76587808, BRD-K76896292, BRD-K93480852, BRD-K98991361, INK-128, MLS-0327420.0002, MW-Ras9, NCGC00182845-01, Sertraline, Valproic Acid, BRD-K04853698, BRD-K74761218, Dasatinib, Geldanamycin, and JW-7-24-1.

38. The method of claim 37, wherein the one or more modulators are selected from the group consisting of Trichostatin A, BRD-K30523950, CI 976, Rolipram, BAS 00535043. BRD-K50177987, BRD-K76568384, 2541665-P2, BRD-K59488055, NCGC00182823-01, BRD-K21565985, BRD-K55612480, BRD-K61217870, BRD-K98991361, Sertraline, Dasatinib, Geldanamycin, JW-7-24-1, Wortmannin, Idazoxan Hydrochloride, MLS-0327420.0002, AZD8055, BRD-K90999434, NSC 23766, Teniposide, BRD-K34495954, BRD-K63326650, BRD-K76896292, and BRD-K74761218.
